(19) United States
(12) Patent Application Publication
(10) Pub. No.: US 2021/0333285 A1
(43) Pub. Date: Oct. 28, 2021

(54) DETECTION OF PHOSPHOKINASE SIGNATURES

(71) Applicant: The Regents of the University of California, Oakland, CA (US)

(72) Inventors: Jean-Philippe COPPE, San Francisco, CA (US); Laura J. VAN'T VEER, San Francisco, CA (US)

(21) Appl. No.: 17/046,986

(22) PCT Filed: Apr. 12, 2019
(86) PCT No.: PCT/US2019/027201

§ 371 Date: Oct. 12, 2020

Related U.S. Application Data
(60) Provisional application No. 62/657,620, filed on Apr. 13, 2018.

(51) Int. Cl. G01N 33/68; C12N 9/12

(52) U.S. Cl. CPC G01N 33/6803; C12N 9/12

(57) ABSTRACT

The disclosure provides methods and compositions for determining the kinase activity profile of a biological sample, e.g., that contains multiple kinases.

### CROSS-REFERENCE TO RELATED APPLICATIONS

This application claims priority benefit of U.S. provisional application no. 62/657,620, filed Apr. 13, 2018, which is incorporated by reference in its entirely for all purposes.

### BACKGROUND OF THE INVENTION

In a functional sense, cancer is a proteomic disease that arises from selectively diverted signaling pathways^{1,2}. Kinase phosphorylation cascades function as adaptive networks that are re-wired by oncogenic processes³⁻⁷. Half of all cancer-drugs that are FDA-approved or in clinical trials target hyperactive kinases, such as BCR-ABL, BRAF^{V600E} or HER2^{8,9}. While therapeutic decisions increasingly rely on the detection of mutated kinase genes or aberrantly expressed/phosphorylated proteins, few experimental platforms are practical enough to directly and comprehensively monitor the activity of kinase enzymes¹⁰, and thus the key actionable dependencies of tumors remain often unclear^{11,12}. A technology capable of identifying the phospho-catalytic signatures of kinases in biological samples could benefit the bio-medical field and improve therapeutic guidance.

Proteomic detection systems use the phosphorylatable regions of proteins to infer kinase activity. Antibody-based assays measure (phospho-)protein levels, which depend on availability and specificity of antibodies^{1,13-15}. Mass spectrometry techniques¹⁶⁻²¹, sometimes combined with kinase inhibitors²²⁻²⁵, allow detection of raw amounts of (phospho-)proteins, but remain restricted due to cost, equipment and protocols. Alternatively, generic amino-acid sequences are used as individual biochemical probes to directly detect the phospho-catalytic activity of kinases in radioactive-labeling assays, microfluidic electrophoresis systems, ATP-consumption tests, hybrid peptide/phospho-antibody platforms, or SPR and FRET techniques²⁶⁻³³. Readouts from these approaches, however, rely on broad-spectrum consensus peptides originally designed for one-probe-to-many-kinases detection methods, well suited for pharmacological drug screens, but not intended to specifically identify or differentiate between kinases' activity in biological extracts.

### BRIEF SUMMARY OF SOME ASPECTS FO THE DISCLOSURE

Provided herein is a new technological resource to distinguish and measure the phospho-catalytic activity of many kinases in parallel. This strategy relies on collections of peptide probes that are derived from the biological target sites of kinases^{34,35}, and are physically used as distinct combinatorial sets of sensors to monitor the activity of kinases in samples. The technology is modular by design: users can adapt probe libraries and assay conditions to their needs. Using a proof-of-concept 228-peptide library, computational methods to analyze phospho-catalytic signatures established from high throughput ATP-consumption measurements are provided in the examples section, which illustrate the present invention. Further, aspects of the invention relate to identification of kinase targets in specific types of cancer. For example, an analysis of BRAF^{V600E} tumors for kinase activity is provided as an illustration. BRAF^{V600E} colorectal cancers (CRC) and melanomas (MEL) remain clinical challenges, with poor prognosis and mostly palliative treatment options, that likely involve kinase-signaling pathways, which can hold the key to new therapeutic opportunities. In one aspect, this approach is used to identify new druggable kinase nodes that drive the unresponsiveness of CRC and MEL to anti-BRAF^{V600E} therapy in cell models and patient tumors. Additional benefits of the present invention will also be apparent to one of skill in the art, e.g., as discussed in the EXAMPLES section.

In some aspects, the disclosure is thus based, in part, on a high-throughput system to measure the activity of kinase enzymes using their biological peptide targets as phospho-sensors. In some embodiments, the disclosure provides a target peptide set to detect the activity of multiple kinase/kinase families, including, e.g., ABL, AKT, CDK, EGFR, GSK3B, MAPK and SRC, using a multiplex assay, e.g., an ATP-consumption screen, to identify the activity of kinases of interest to be evaluated, e.g., kinase activities in cancer cells. Further, in some aspects, the disclosure provides methods and compositions for the analysis of drug sensitivity, e.g., drug-sensitivity to BRAFV600E-targeted therapy in colorectal cancer; and identifies phosphocatalytic signatures of melanomas for designing and implementing therapies.

### BRIEF DESCRIPTION OF THE FIGURES

FIG. 1. Libraries of biological peptides function as combinatorial sensors to identify, differentiate, and measure the phosphorylation activity of kinases.

(a) Schematic of the assay procedure.
(b) Unsupervised hierarchical clustering of phospho-catalytic activity signatures of 25 recombinant kinases established from 228 peptides. For each experimental run, the average value of ATP consumption across the 228 peptides was used for internal normalization. The kinase activity per-peptide was then calculated as the difference in ATP consumption between individual peptide-derived values and the overall internal mean. Next, peptide-specific activity values were averaged across independent repeats to establish the phospho-activity signature of each kinase for all peptide sensors. Finally, the 228-peptide-derived phospho-catalytic signatures of the 25 recombinant kinases were analyzed using unsupervised hierarchical clustering. Phospho-catalytic profiles are color-coded based on the relative level of activity measured in presence of each peptide, from blue for low-or-no activity, to white for intermediate-or-mean activity, to red for high phospho-catalytic activity. The red/gold/grey peptide color-key on the right side of the phospho-catalytic heatmap indicates the origin of peptides (biological, generic positive control, or random). The number of independent experimental repeats per kinase (n) is listed underneath the heatmap. The same concentration of recombinant kinases was used (see Supplemental Methods).
(c) Pearson-correlation heatmap highlighting the functional relationship between kinase enzymes established from their differential spectrum of 228-peptide-specific phospho-catalytic activities. Kinases are arranged by alphabetical order within their respective families.
(d) Profile of AUC values obtained for an increasing number of randomized sampling combinations of peptide sensors to predict the identity of HCK. AUC values (y-axis) reflect the performance of HT-KAM assay for predicting HCK's identity by comparing the 7×228-peptide phospho-signatures of HCK versus the 113×228-peptide phospho-signatures measured for all other 24 kinases, when relying on one or multiple peptide sensors (x-axis; random peptide sampling of a combination of up to 50 peptides out of 228). The AUC values corresponding to Diagonal Linear Discriminant Analysis (DLDA) classifiers that match HKC's specific all-biological peptide subset is shown as a red dot; HCK's all-generic positive control peptide subset as a gold dot; all-random peptides as a grey dot; exact AUC values and number of peptides for each combination are indicated.
(e) Distinct subsets of peptides can be identified as functional predictors of the differential activity signature of each kinase family. The number and origin of peptides composing the differential phospho-signature of kinases is shown in the bar graph. Peptides were classified as ‘predicted’ (dark shade) or not (i.e. ‘other’; light shade), where ‘predicted’ defines a peptide sequence previously identified in the literature as a target of a given kinase. For a biological peptide, it is an amino acid sequence that corresponds to a known phosphorylatable protein region³⁴, and for a generic positive control peptide it is an amino acid sequence that is commercially available. The AUC of each peptide set per kinase is listed underneath the graph.
(f) Phospho-catalytic profiles measured in presence of the biological peptide targets of AKT1, MAPK1/ERK2 or JAK2 kinase. The average activity per indicated biological peptide across ‘n’ independent experiments is color-coded blue-to-white-to-red from low-to-medium-to-high kinase activity. Biological peptide probes are organized by decreasing phospho-catalytic activity per kinase. Each phospho-catalytic heatmap is shown side-by-side with a grey-scale of significance (p-values for a pairwise t-test comparing the activity measured from ‘n’ independent experimental runs for each biological peptide vs. the average activity measured with 5-random peptides). Average phospho-catalytic activities obtained from four distinct control peptide groups are shown for comparison at the bottom of each panel (random, Y/S/T-free, reference, or generic positive control).
(g) Box plots showing that the subsets of biological peptides specific to each kinase are associated with significantly higher levels of phosphorylation activities than activities measured from the pool of 5-random peptides. Box plot distribution of data displays minimum, first quartile, median, third quartile, and maximum. The difference in kinase activity experimentally measured with biological vs. reference peptides was highly significant (p<2E-100; left vs. right set of box plots).
(h) Functional clustering of kinases based on the 151-biological peptides included in the assay. The analysis is based on all HT-KAM readouts, but only phospho-activity values derived from the 151 biological peptides were used for ranking analysis. Kinases belonging to distant genetic families are distinguishable. ABL and SRC kinases from non-receptor tyrosine kinase families are identified as functionally related. Individual kinases of known distant biological/biochemical relationship with the rest of their genetic families can be singled out (e.g. ABL1^{T315I}; AKT2; MAPK14; SRMS).
(i) AUC forest-plots comparing the specificity/sensitivity of biological (red) vs. generic control positive (gold) vs. random (grey) peptide subsets. Biological peptides behave as good functional sensors of kinases, and the identity of a kinase can be effectively predicted from the phospho-signature monitored with its subset of biological peptides.
(j) Enzymatic activity of ABL1 measured in presence of its 11-biological peptides, and either without treatment (left column; UNT) or treated with serial dilutions of ABL1-targeting inhibitors imatinib and dasatinib (2 sets of 3 columns on the right; concentrations indicated at the top).
(k) Measurable effects of inhibitors on the activity of ABL1, ABL1^{T315I}, LYNA, AKT1 kinases using their biological peptides as reporters. Shifts in activity are represented as the average (colored dot)+/−StDev of the change in ATP consumption across all biological peptides in comparison to untreated control. While the activity of ABL1 was most reduced by these inhibitors, the anticipated specific effects of inhibitors was apparent by their relative low or lack of effect on ABL1^{T315I}, LYN A and AKT1 kinases (e.g. LYN A was affected by higher doses of Dasatinib). Staurosporine was used as a non-specific control of kinase activity inhibition.

FIG. 2. Mapping the phospho-catalytic signatures of cancer cells in their native or drug-treated/resistant states, identify their specific kinase dependencies and reveal their differential vulnerabilities to single and combinatorial kinase-targeting therapies.

(a) Identification of kinases known to participate in the response of BRAF^{V600}E CRC cells (WiDr) to BRAF^{V600E}-targeting therapy (VEM). Western blots for total and phospho-protein MEK1/2, ERK1/2 and EGFR are shown as controls on the left. Change in activity of these kinases after VEM treatment was measured using their respective subsets of biological peptides (shown as heatmaps and bar graphs). Briefly, for each experimental run, the average value of ATP consumption across the 228 peptides and 14 data-points from cell extract alone (i.e. established from 14 peptide-free control wells per 384-well plate) was used for internal normalization. Each sample's kinase activity per-peptide was then calculated as the difference in ATP consumption between individual peptide-derived read outs and the internal mean. Next, the peptide-specific activity values were averaged across independent repeats (3 independent biological replicates tested in 4 independent technical replicates). Finally, the difference in phosphorylation activity per peptide between VEM and UNT profiles was calculated across all 228 peptides. Results are represented as a series of kinase-focused heatmaps using their particular subset of biological peptides (top right panel). Each colored bar represents the differential activity in presence of a biological peptide of the indicated kinase, ranging from blue-to-white-to-red to respectively indicate lower-to-unchanged-to-higher activity after VEM treatment. The number of biological peptide sensors per kinase is indicated underneath the name of the kinase. A relative cumulative index of activity for each kinase was plotted as an indicator of the differential kinase signatures found in VEM-treated samples versus their control counterpart (bar plot on the bottom right; average of differential activity values across all kinase-specific biological peptides, divided by the number of peptide sensors). Significance is measured in pairwise Student t-test comparisons between VEM and UNT across all runs (*/#: p<0.05; **/##: p<0.01), and either including all biological peptide sensors (*/**), or 75% of peptides that followed the main activity trend (#/##) to elude cross-reaction effects due to parallel feedback activation loops such as AKT or EGFR as illustrated from the biological peptide targets of ERK2 that paradoxically display higher activity (red bars at the top of the ERK2-heatmap) but relate to proteins directly involved in mechanism of therapeutic resistance, such as the EGFR-pathway^{4,36} or TGFBR-pathway⁴⁷.
(b) Identification of new kinases that mediate intrinsic resistance to BRAF^{V600E}-targeted therapy in WiDr CRC cells.
(c) Validation of changes in activity of kinase enzymes using western blot. For example, increased phospho-S473 AKT1 relates to increased AKT1 activity, while increased phospho-S9 GSK3B foretells decreased GSK3B activity.
(d) Response of WiDr cells to combinatorial-targeted therapies. Survival graphs show cell growth sensitivity profiles to dual targeting of BRAF^{V600E}+AKT1 or PDPK1 or PRKCA. Table insets provide characteristics of response to drug combinations: C.I.=combination index following Loewe Additivity model and measured as the average growth inhibition for VEM52 uM and 2nd drug≤GI50 concentration (arbitrary threshold: synergy CI≤0.6; additivity 0.6<CI≤1.0); D.I.=drug interaction (two-way ANOVA p-val). Drug responses serve as a validation of kinase activity signatures found by HT-KAM screening, as well as a discovery of new vulnerabilities.
(e) Peptide phosphorylation activity signature of cancer cell lines. For each experimental run, the average value of ATP consumption in sample-containing wells measured across 228 peptides and 14 peptide-free controls, was used for internal normalization. The activity per-peptide was then calculated as the difference in ATP consumption between individual peptide-derived read outs and the internal mean. Next, phosphorylation activity values measured for each peptide were averaged from ≥3 independent replicates for each cell line. Finally, the phospho-catalytic activity signatures measured across the 228-peptide sensors were subjected to unsupervised hierarchical clustering. Phospho-catalytic activities are color-coded based on the relative level of activity measured in presence of each peptide for each cell line, from blue for low activity, to white for intermediate-or-mean activity, to red for high activity. The peptide class is indicated as a red/gold/grey color streak on the right side of the heatmap.
(f) Kinase activity signature of cancer cell lines. For each cell line, the activity of kinases was calculated as the average of the phosphorylation activities measured in presence of their respective biological peptide subsets. The profiles of 60 individual kinases or kinase families detected with ≥4 biological peptides are shown. Unsupervised hierarchical clustering was applied across both kinases and cell lines, and color-coded as a blue-to-black-to-yellow scale from low-to-medium-to-high activity. Profiles highlight the heterogeneity of kinases' activity across cancer cells. Control for ATP levels and protein concentrations across cells, and validation of phospho-signatures using complementary computational analysis, immuno-blotting, or drug treatment responses are available in FIGS. 18-22.
(g) Comparison of kinase activity and drug sensitivity in two phenotypically distinct BRAF^{V600E} cell lines: A375 (melanoma) vs. WiDr (colorectal), which are respectively considered to be inherently sensitive and resistant to BRAF-therapy. The activity of 10 different kinases was mean-centered between cell lines (y-axis) and compared to their GI50's (x-axis; drug concentration causing 50% inhibition of cell growth in 3-day assays) for 14 inhibitors. Color-coded drug names are indicated underneath each graph. Validation using a second, alternative drug was tested for four of the kinases. All drug concentrations are in uM, except dinaciclib and trametinib in nM.
(h) Graphical correlation of kinase activity with drug sensitivity. Kinase activity values (y-axis) correspond to activities shown in (c). The x-axis shows the sensitivity of A375 calculated as the Log₁₀ of GI50(WiDr)/GI50(A375).

FIG. 3. Mapping the phospho-catalytic signatures of melanoma tumors reveals druggable kinases predictive of poor outcome and vemurafenib-unresponsiveness in BRAF^{V600E}U patients.

(a) Schematic of the procedure.
(b) Peptide phosphorylation signatures of patient tissues. Data analysis followed the same steps as for cancer cell extracts. HT-KAM data were clustered using Euclidean distance and ward linkage. Phospho-catalytic activity and peptide annotation are color-coded as indicated in the bottom right legend. Retrospective knowledge of survival outcome, recurrence and treatment-resistance are indicated above each map, along with their BRAF^{V600E} mutational status. Within the deceased group, patients #4-5-9 were VEM-resistant (red squares), and patients #2-4-8 were ipilimumab-resistant (i.e. refractory to anti-CTLA4 immuno-therapy); see FIGS. 23-24 for details.
(c) Kinase activity signatures of tumor tissues. Analysis followed the same steps used to deconvolute the profiles of cell lines. For each tumor, the activity of kinases was calculated as the average of phosphorylation activities measured in presence of their respective biological peptide subsets (only kinases detected with ≥4 biological peptides are shown). Kinase activity profiles were mean-centered per tumor and then per kinase across samples. Semi-supervised hierarchical clustering was applied across kinases (tree on the left; Euclidean distance), while maintaining the order of patients established in (b). Kinases' catalytic activity is color-coded as a blue-to-black-to-yellow scale of relative low-to-high activity. Particular kinases are indicated by an arrow/letter on the right, and underline the balance between up/down-activation of proto-oncogenic kinases (RPS6KB, PIM, AKT) versus tumor suppressor kinase (GSK3B), either within each patient (top vs. bottom; e.g. VEM-resistant BRAFV600E mutated melanomas), or between patient groups (left vs. right; e.g. good vs. poor outcome).
(d) Table of kinases whose biological peptides are significantly represented among most differential peptides associated with survival outcome. The middle column shows significance based on enrichment analysis EASE—Fisher one-sided test using 34 out of 228 peptide sensors (p<0.05 as significance cut-off). The right column provides significance values (FDR-corrected Student t-test) using all (unselected) biological peptides per kinase subset, and comparing all experimental runs between surviving and deceased groups.
(e) Biological peptide-derived activity signatures of AKT, PIM, RPS6KB and GSK3B kinases averaged across tumors from VEM-resistant patients #4,5,9.
(f) Response of BRAF^{V600E} melanoma cell lines to drugs that target vulnerabilities identified by HT-KAM assay. We chose to target AKT, PIM, RPS6KB and GSK3B kinases based on the kinase profiles of VEM-resistant patients shown in panels (c-e). The growth of A375 and Sk-Mel-28 cells was tested in presence of a range of concentrations of VEM (horizontal axis; concentrations indicated at the top) combined with a 2^{nd} inhibitor (vertical axis; kinase target, drug name, range of concentrations indicated on the left) in 3-day survival assays. We elected to use Sk-Mel-28 cells because they are inherently highly resilient to BRAF-therapy. The relative scale of growth inhibition (GI) is color-coded (legend on the top left). GI heatmaps serve as visual indicators of whether the therapeutic index of BRAF-therapy alone can be improved when combined with a 2^{nd} kinase-targeting drug.
(g) Streamlined side-by-side comparison of drugs' effects on cells' response to BRAF^{V600E} targeting. Graphs show the percentage of growth inhibition (y-axis) resulting from combining VEM at various concentrations (x-axis) with a 2^{nd} inhibitor at its GI50 concentration alone (curves of VEM+2nd drug are color-coded; black lines represent the control VEM alone). The 2^{nd} and 4^{th} graph serve as control response profiles obtained from MTOR- and MEK-inhibitor treatment in A375 and Sk-Mel-28. Rectangles highlight the highest achievable response elicited by these various drug-combinations. Percent cell death at GI50 concentrations are provided underneath the graphs.
(h) Characteristics of drug combination effects. D.I.=drug interaction. C.I.=combination index following the Bliss Independence model and measured as the average of all experimental CI values (threshold: synergy CI<0; additivity CI=0; antagonism CI>0)⁴⁸.
(i) Primary tumor cells derived from a VEM-resistant melanoma PDX that displays high level of phospho-RPS6KB1 protein (used as readout of kinase activity), are sensitive to RPS6KB-targeting drug in 3-week colony formation assay.

FIG. 4. Source of peptide probes and arrangement of kinase assay plates.

(a) List of 228 peptides used in the assay. The table provides the connectivity details between kinases and peptide substrates (columns 3-5) for the 151 biological peptides, 14 generic positive control peptides, and 63 reference peptides included in the assay. Peptide ID's and categories are listed in the first two columns. The 25 kinases listed above the color-coded area (right side) are those tested in biochemical assay using purified recombinant kinases.
(b) Numerical-/color-coded connectivity used in table (a). This defines the ‘functional’ relationship between each peptide lane and each tested recombinant kinase shown in table (a). For instance, a peptide/kinase intersection coded 1-black corresponds to a biological peptide predicted from literature to be phosphorylated by the kinase indicated at the top of table. However, this same biological peptide is coded 2-yellow in adjacent columns, which indicates that other kinases are not predicted to phosphorylate this peptide/substrate target sites. Similarly, a peptide/kinase intersection coded 3-orange corresponds to a generic positive control peptide predicted from literature to be phosphorylated by the kinase indicated at the top of table. However, this same generic positive control peptide is coded 4-teal in adjacent columns, which indicates that other kinases are not predicted to phosphorylate this peptide/substrate target sites.
(c-d) Origin of peptide sensors. The functional relationship between kinases and peptides was previously established from computational curation resources built to mine public databases (see: website at http address cancer.ucsf.edu/phosphoatlas; US20120296880^{1,215}). Panel (c) summarizes the curation process and molecular connectivity between kinases, substrate proteins, and phosphorylatable peptide target sequences. Panel (d) is a projection of the coverage of biological peptide targets (left) and generic positive control peptides (right) per kinase across all human kinases/kinase families. Generic positive control peptides were curated from commonly available/advertised single-probe kinase assays or screens. The quantitative and qualitative coverage of peptides per kinase is represented as discs, which are dimensioned based on absolute number of peptide targets per kinase, and color-coded based on uniqueness of peptide targets per kinase.
(e) Biological peptide/kinase connectivity.
(f) Overall arrangement of individual 384-well plates. Besides the 228 peptides, each experimental plate includes: (i) 14 ‘peptide-free’ wells (i.e. sample with buffers but without any peptide), (ii) internal duplicates for a series of peptides-containing wells and peptide-free wells, (iii) assay controls: ATP alone (ATP-loading control), kinase assay buffer alone, and ATP dilution standard.

FIG. 5. Activity signatures established for all recombinant kinases across all peptide sensors and all experiments.

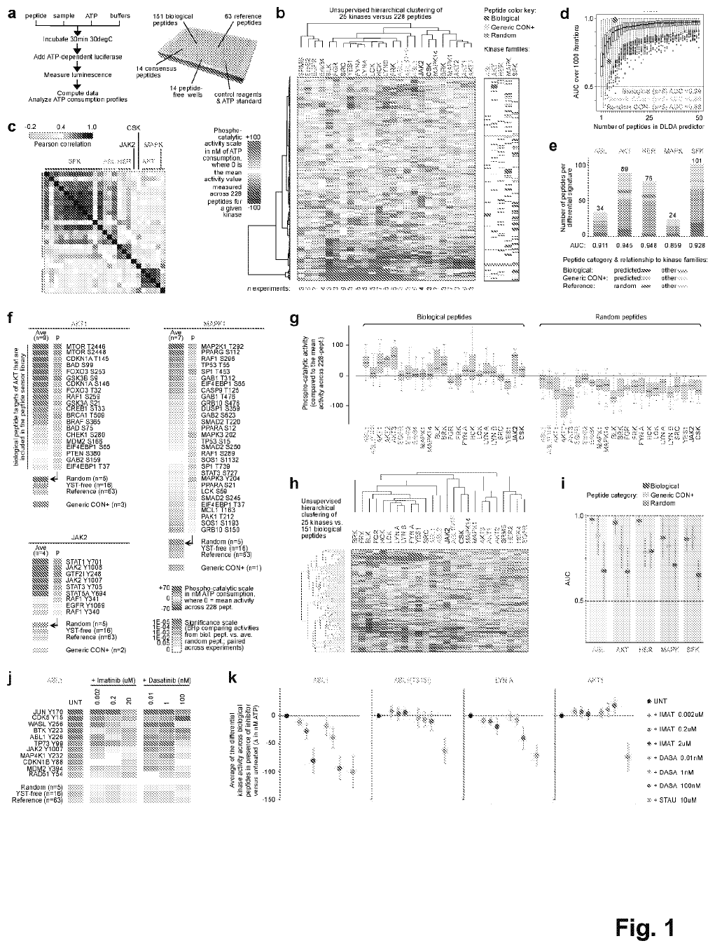

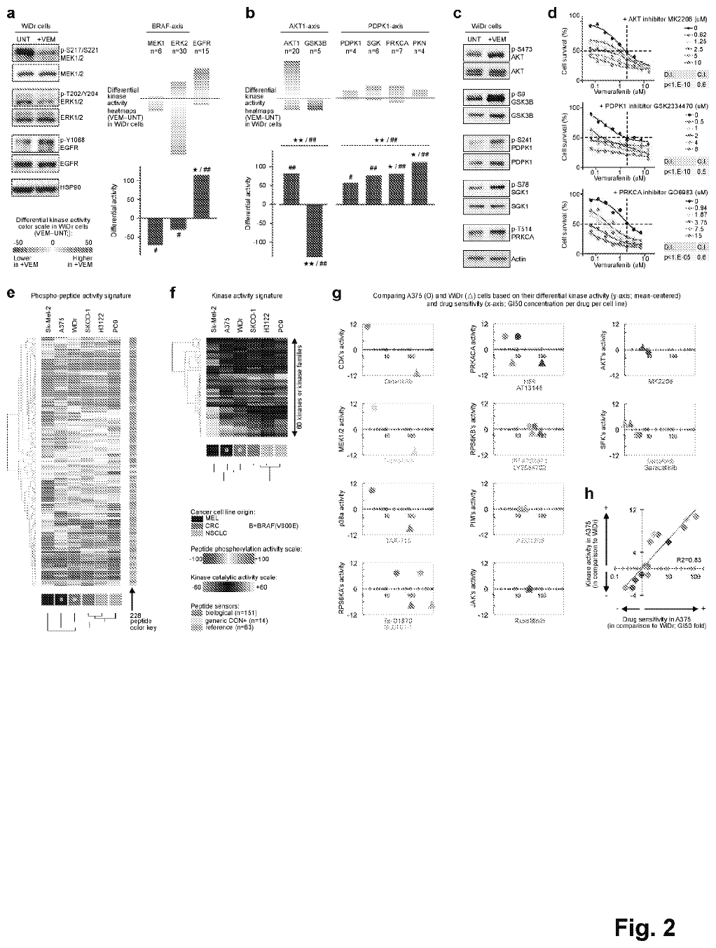

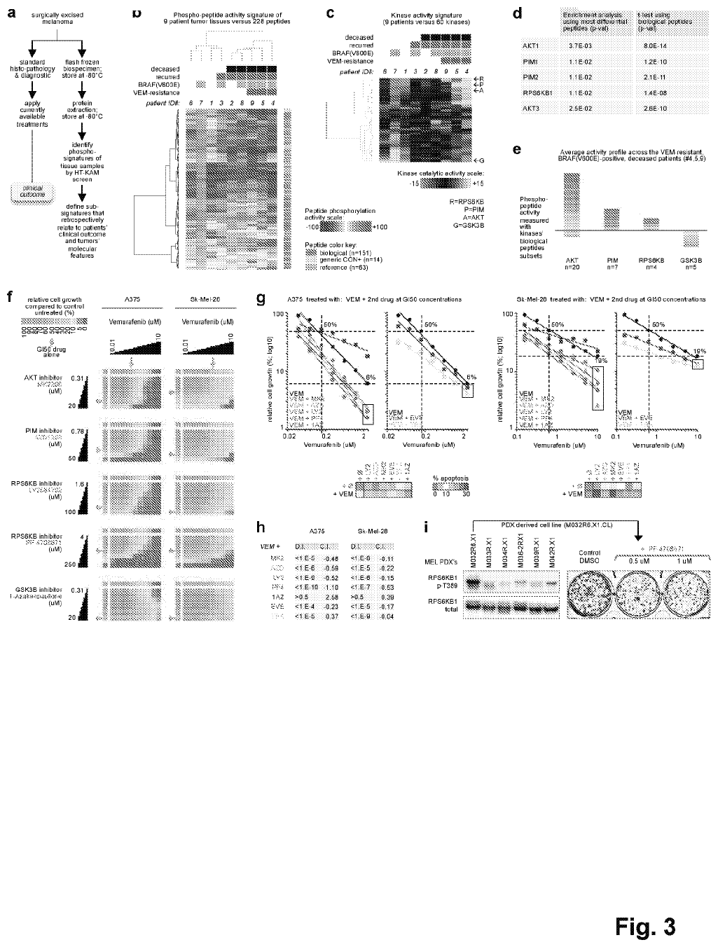

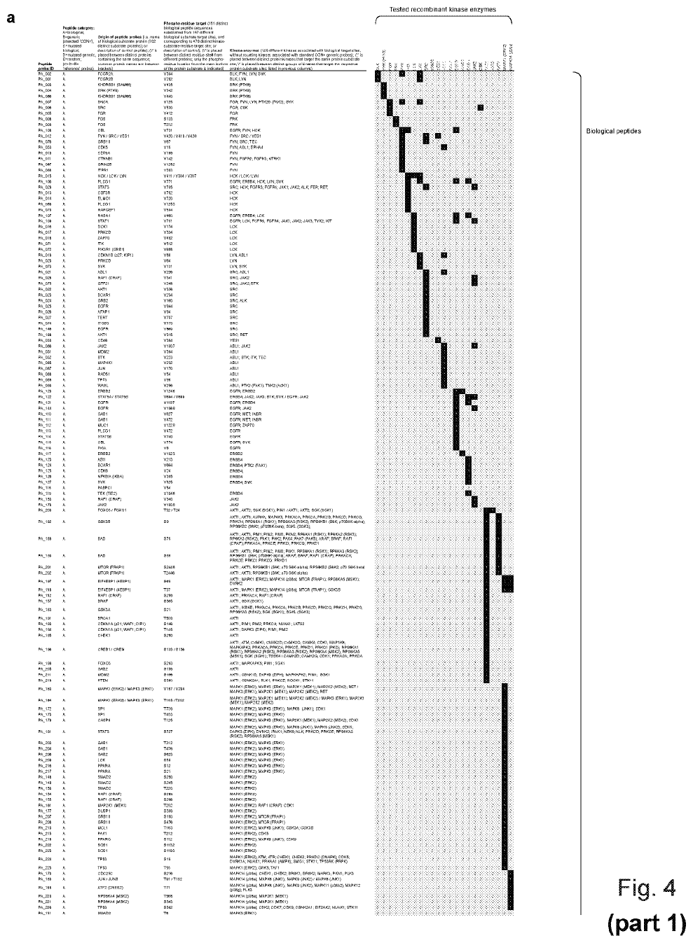

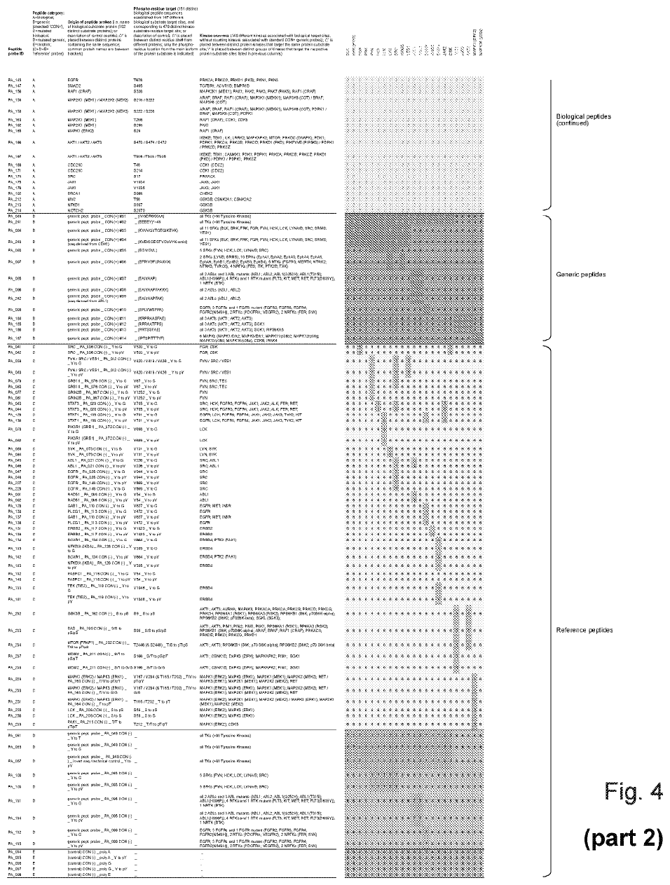

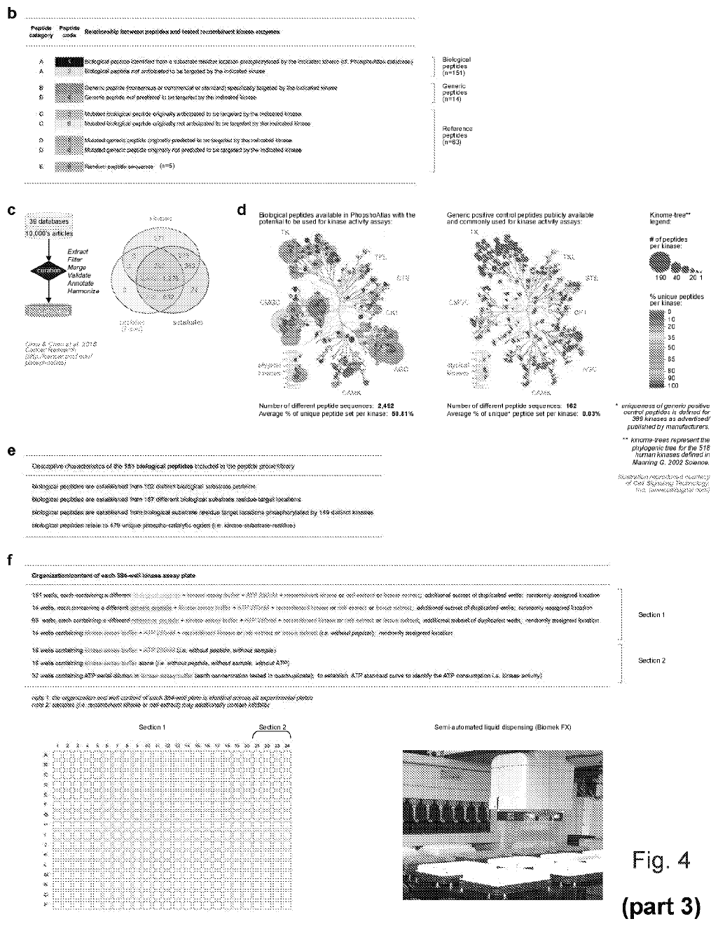

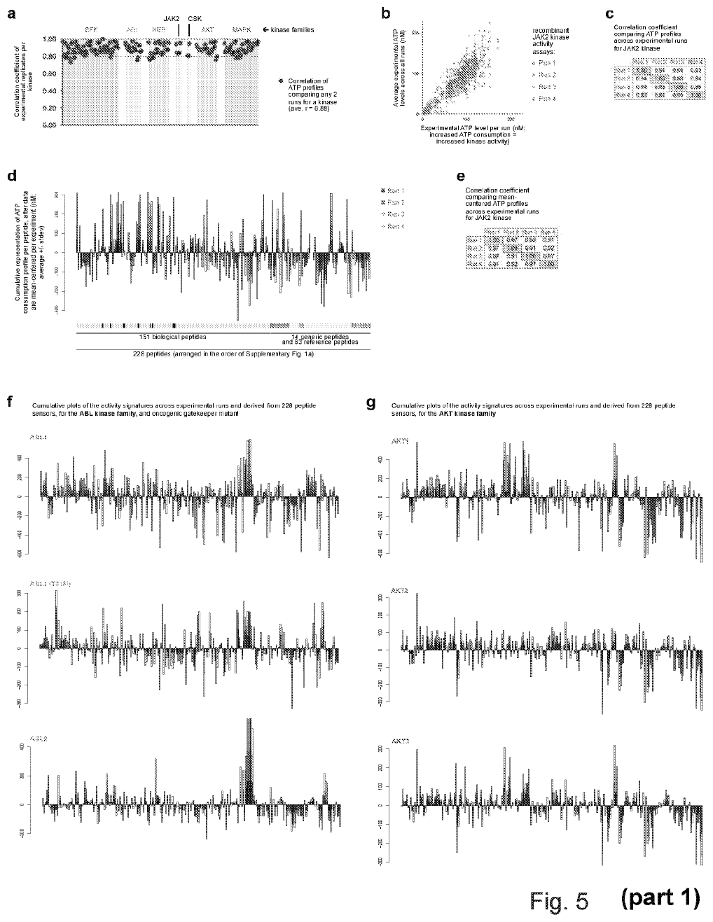

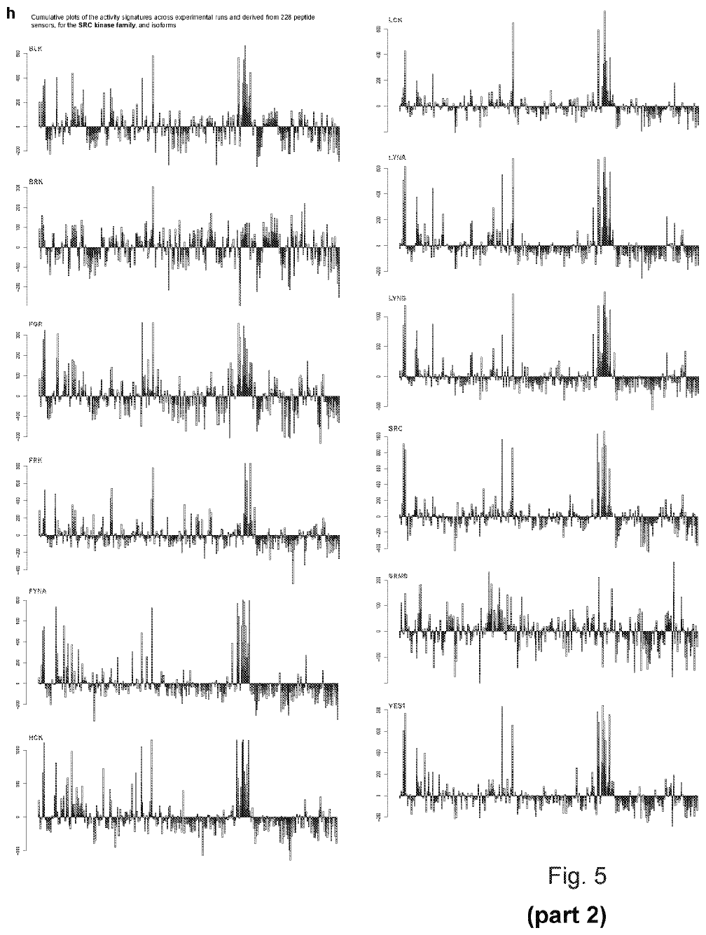

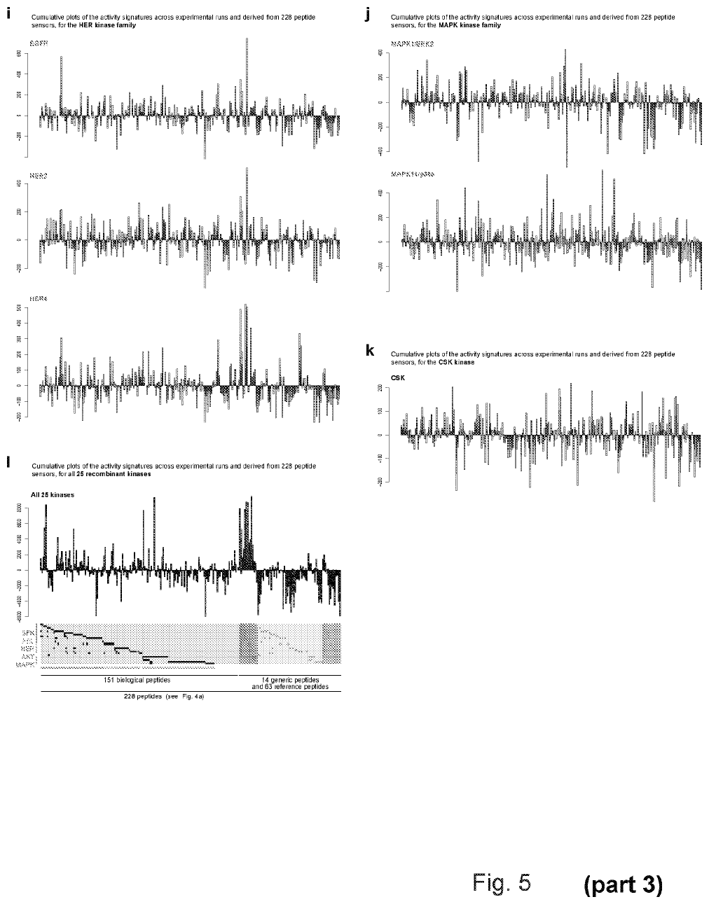

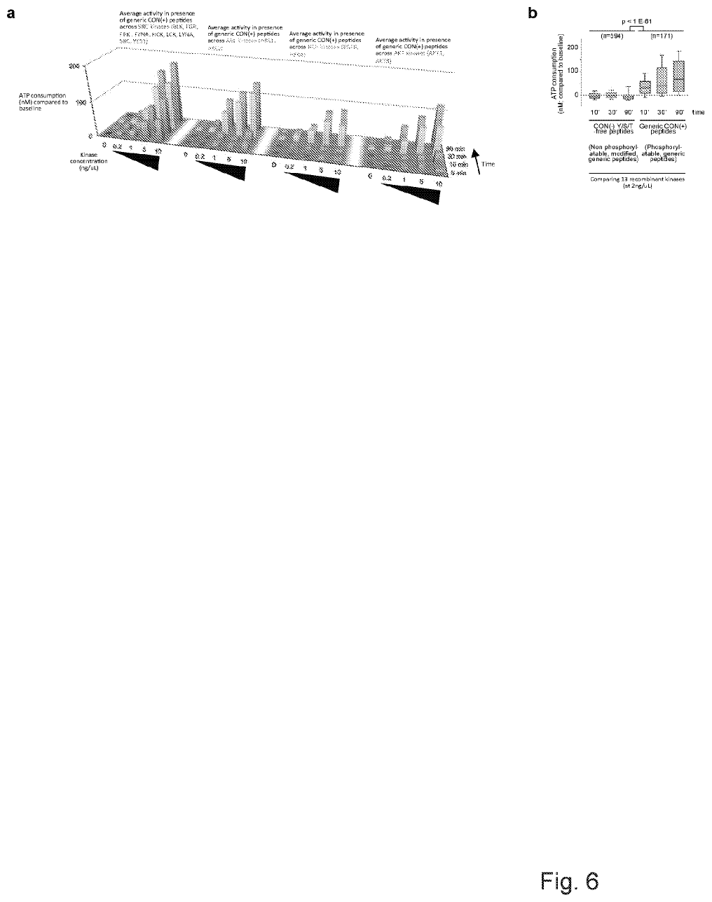

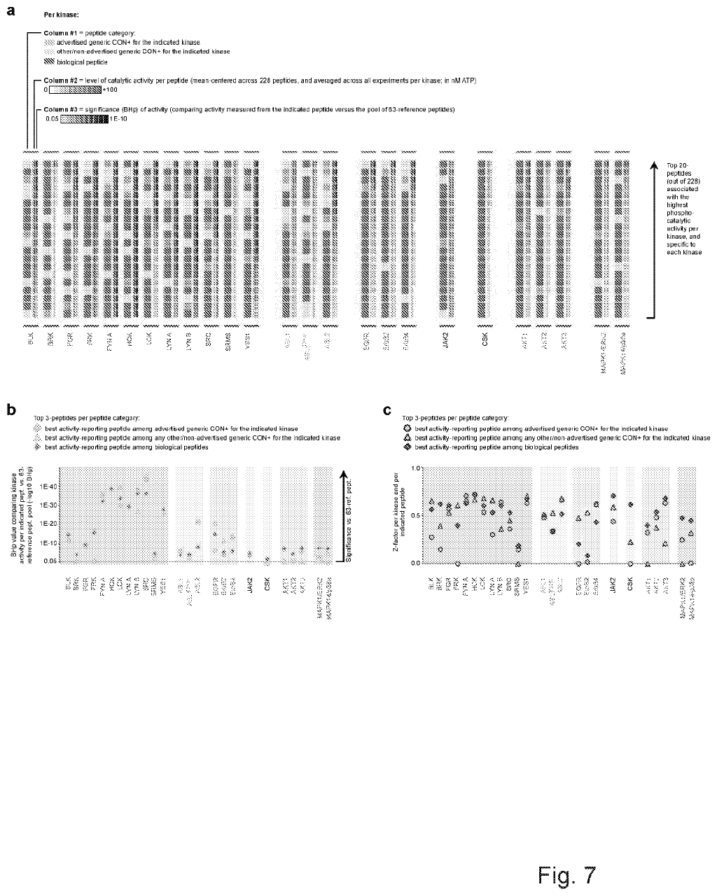

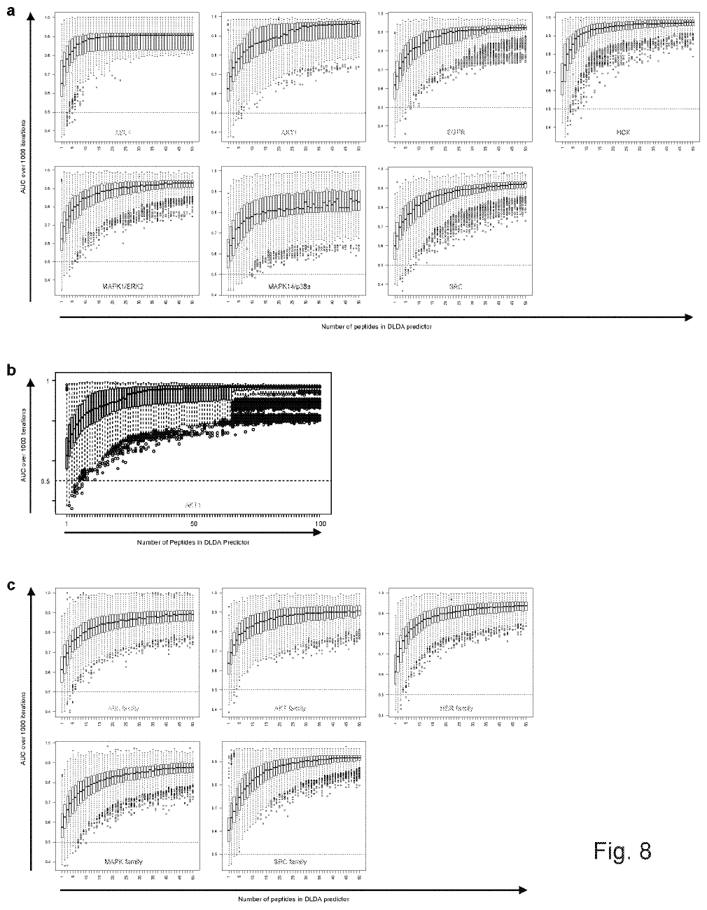

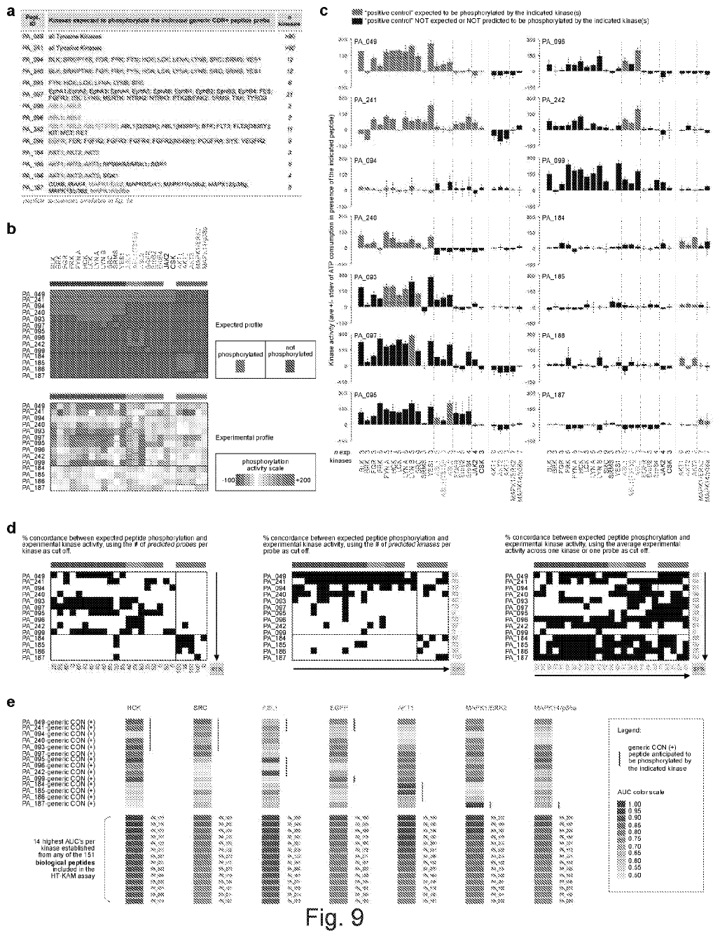

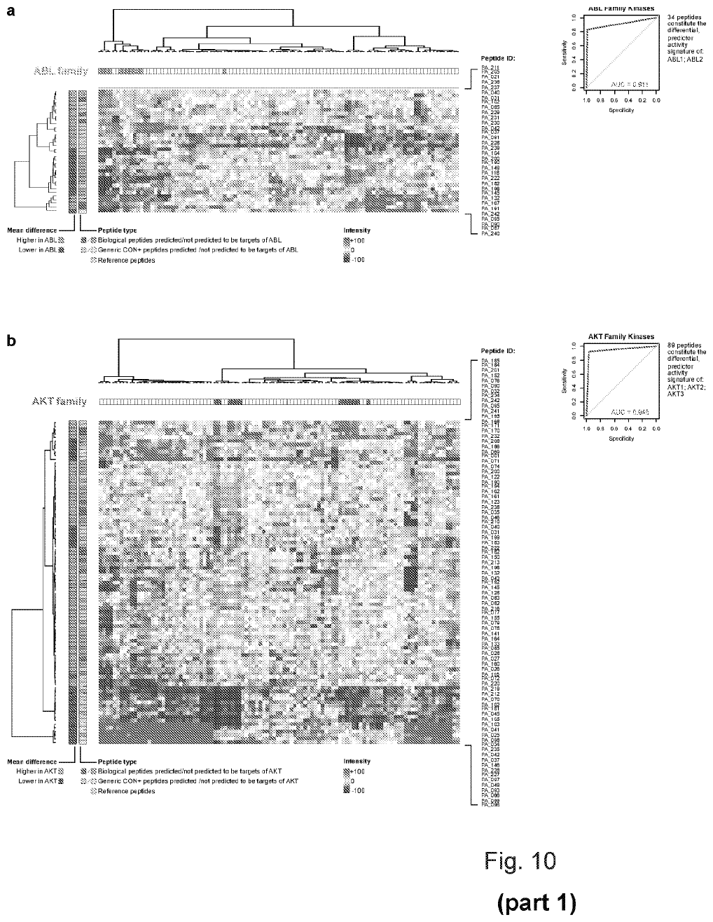

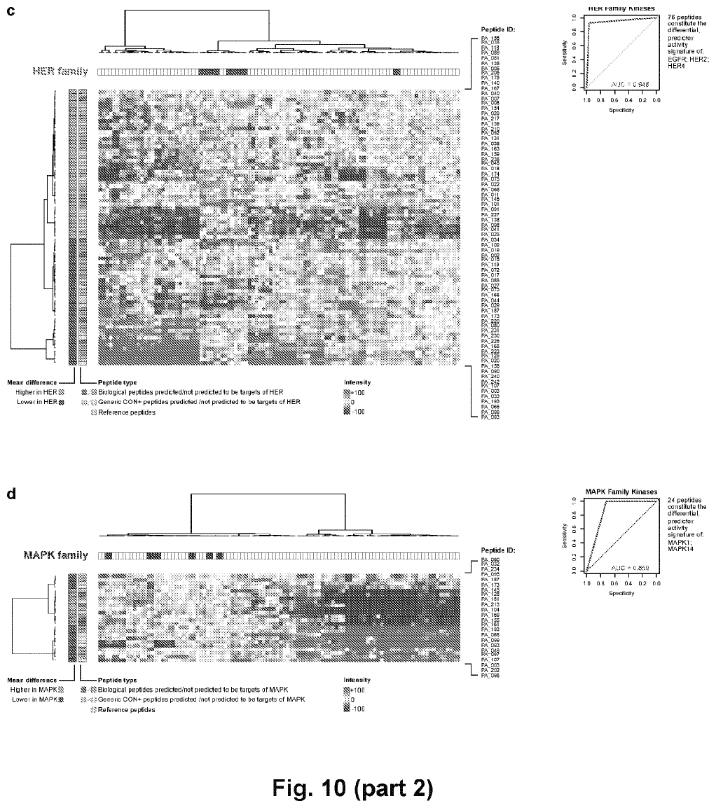

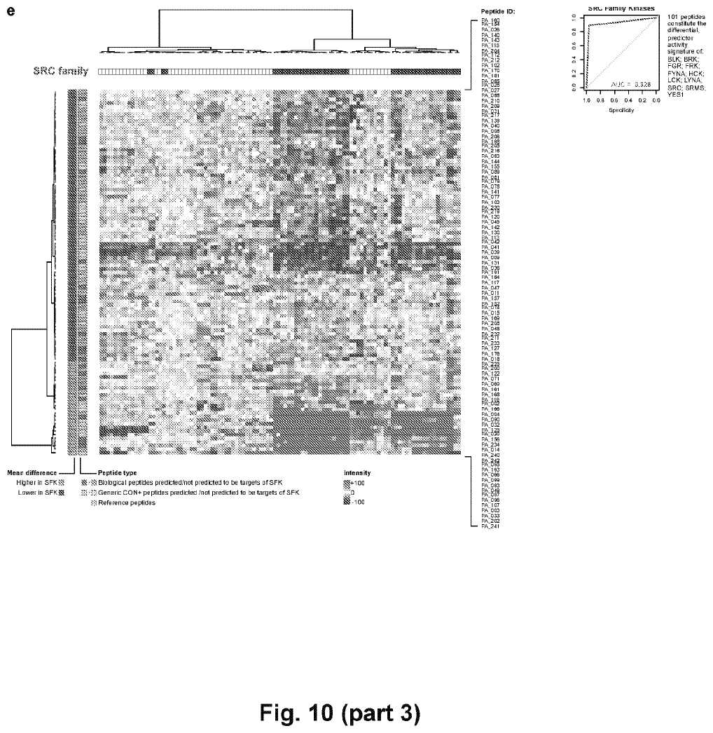

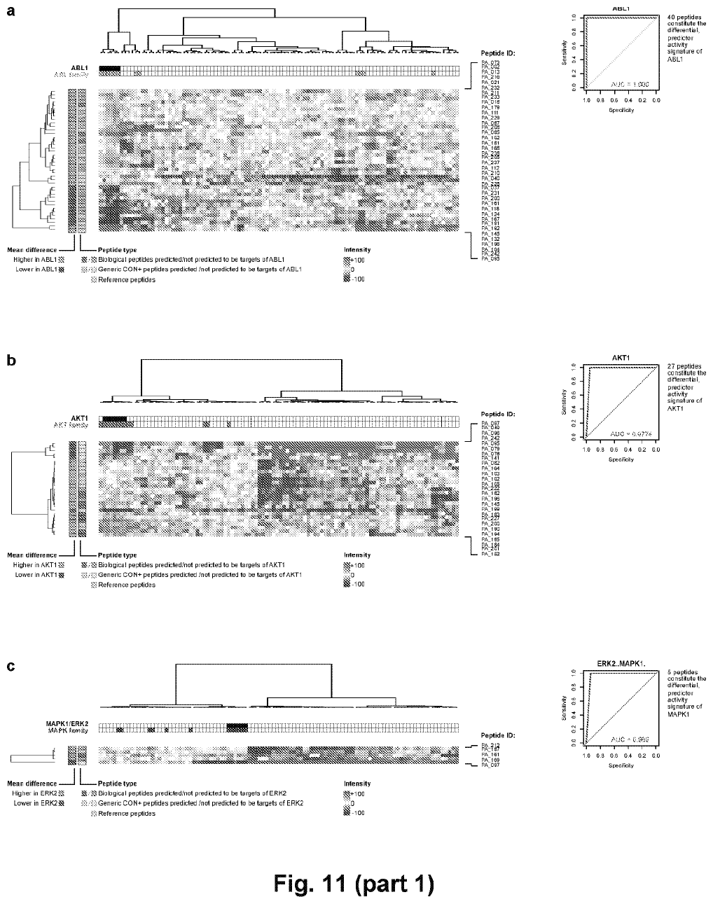

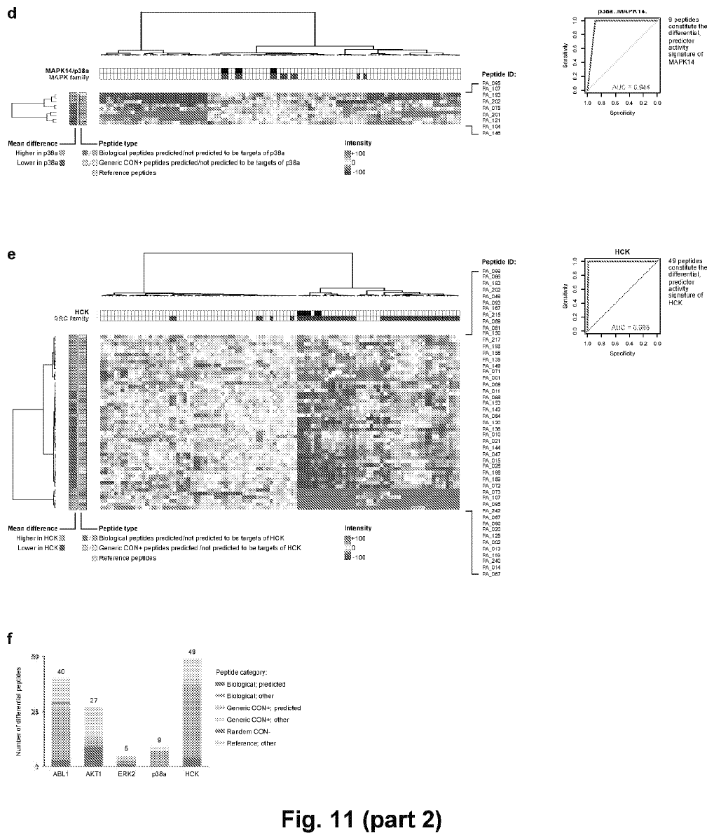

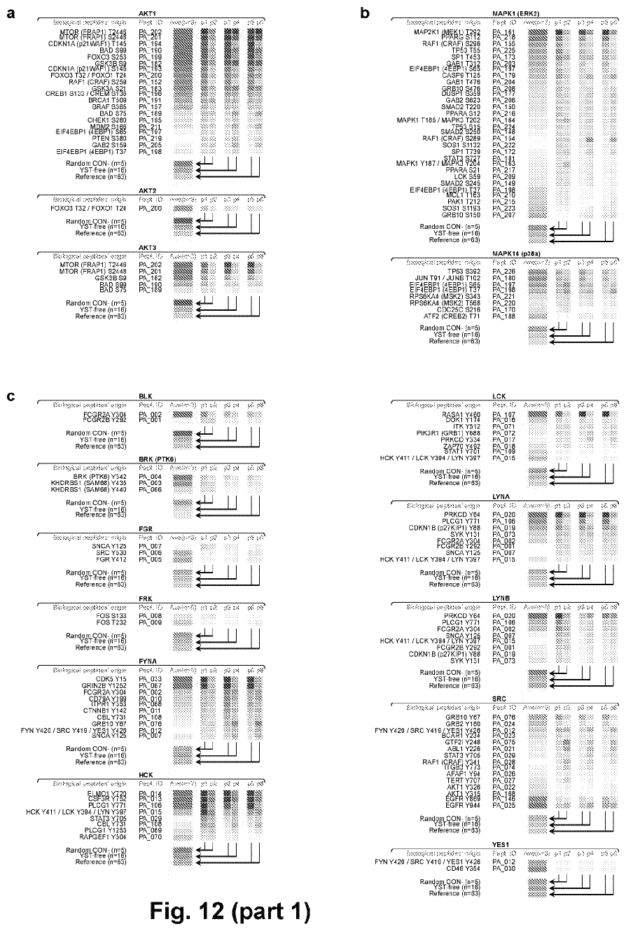

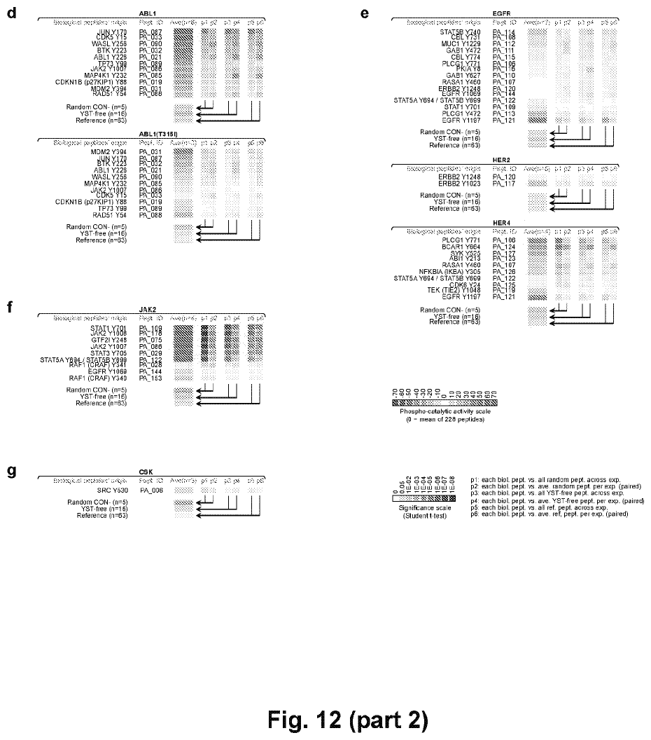

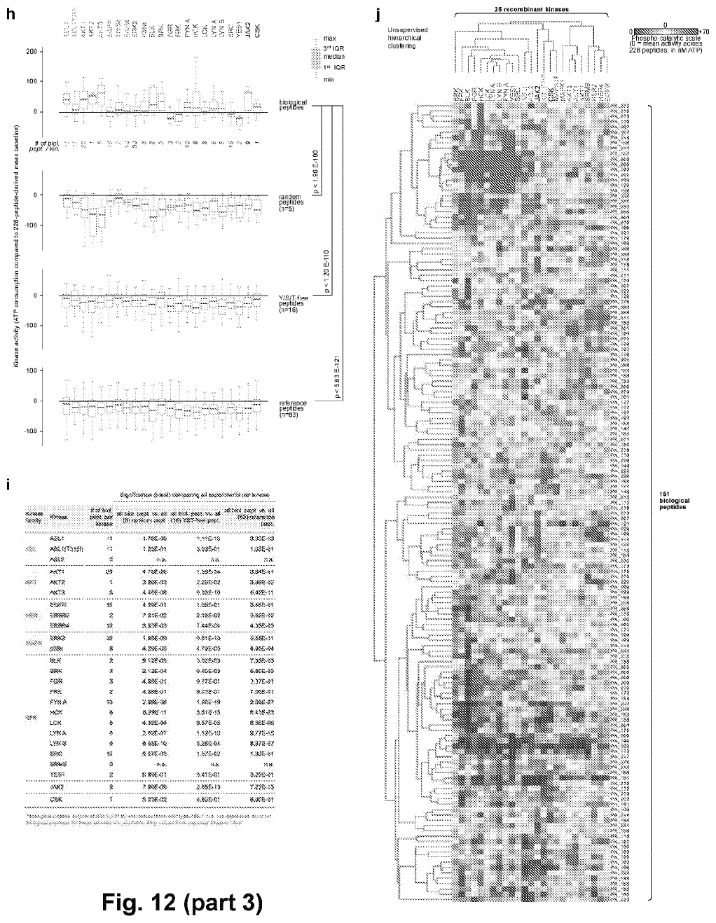

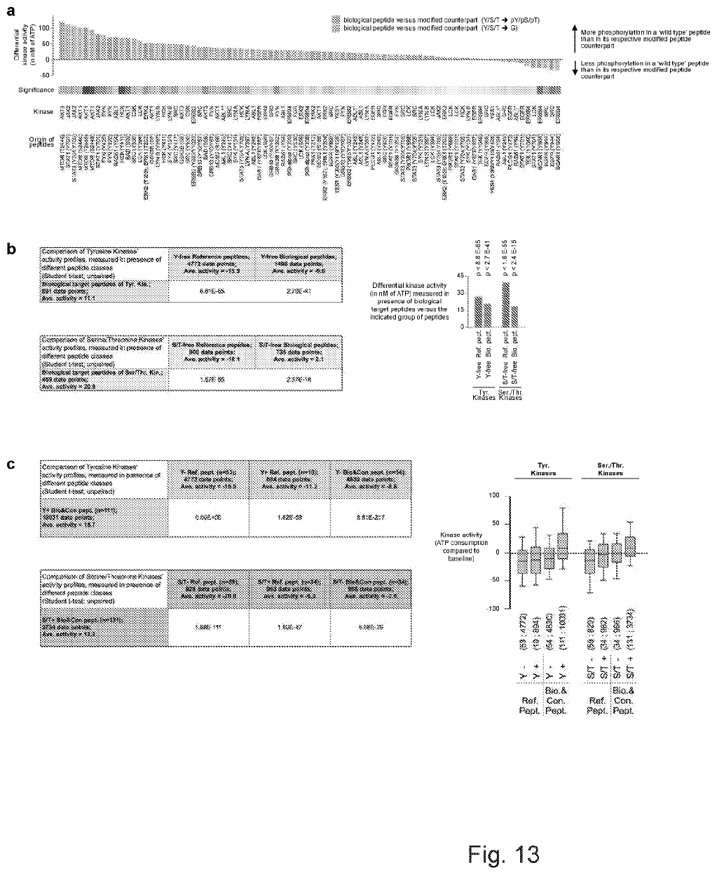

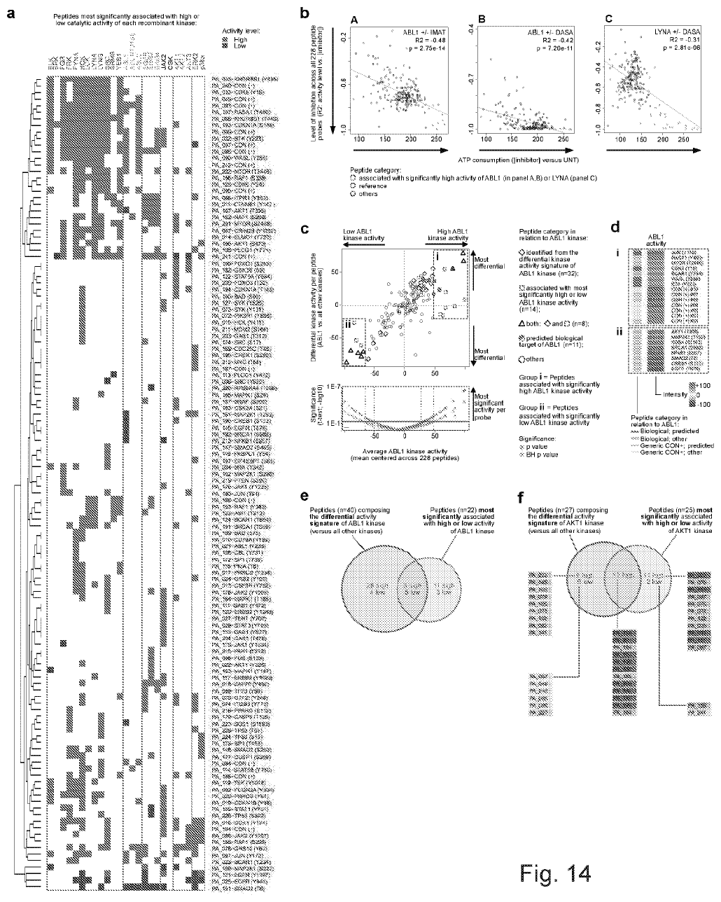

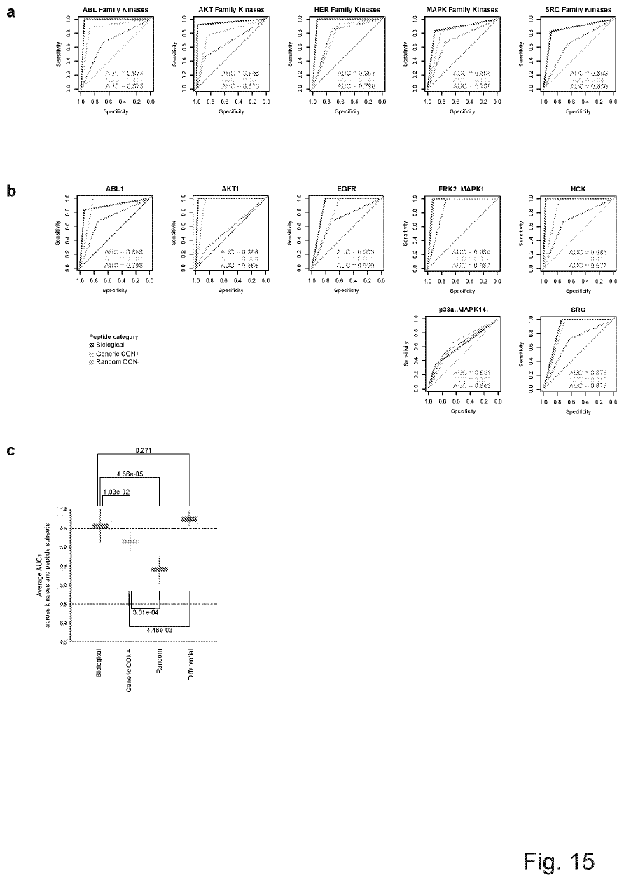

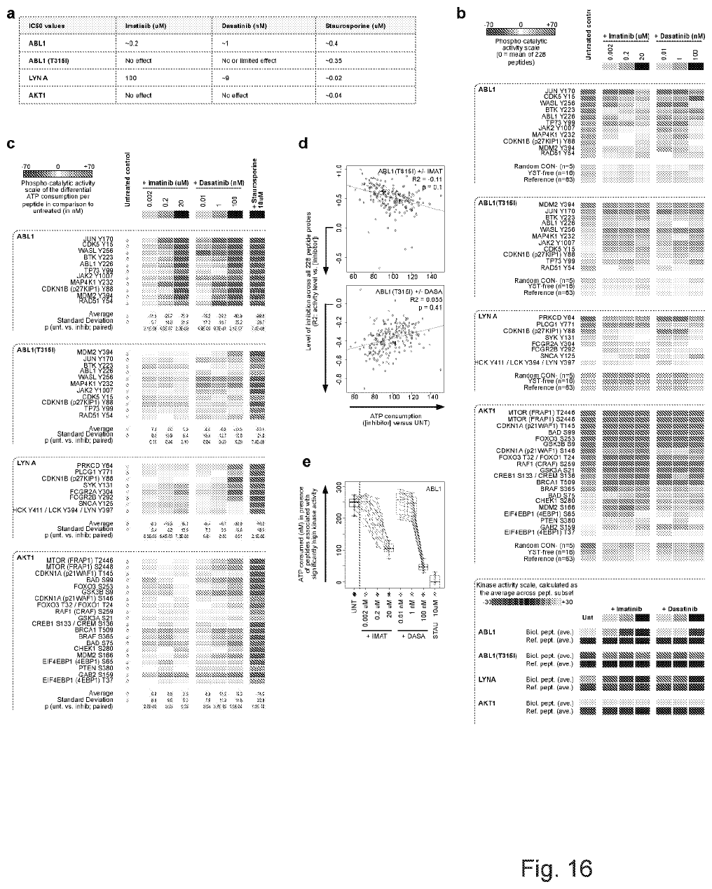

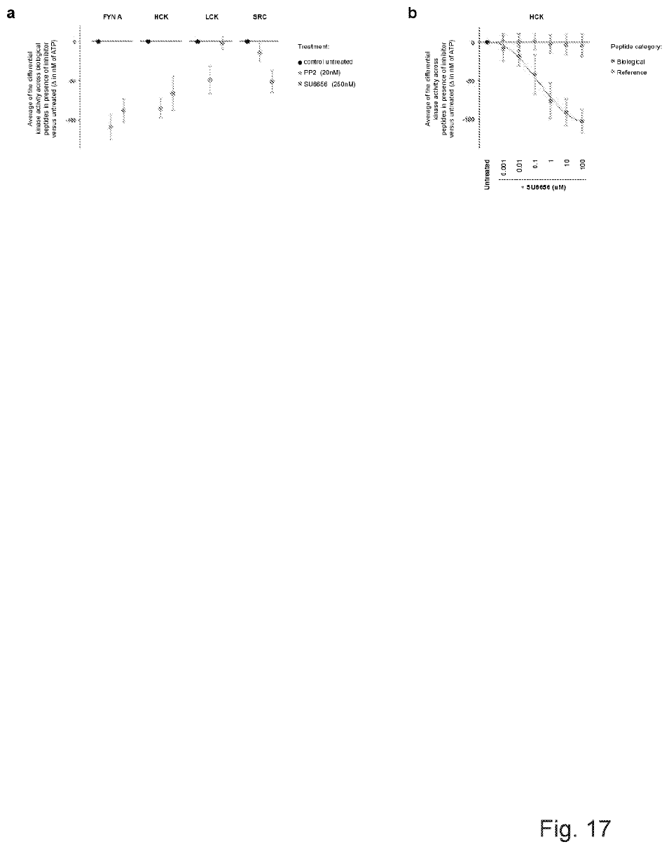

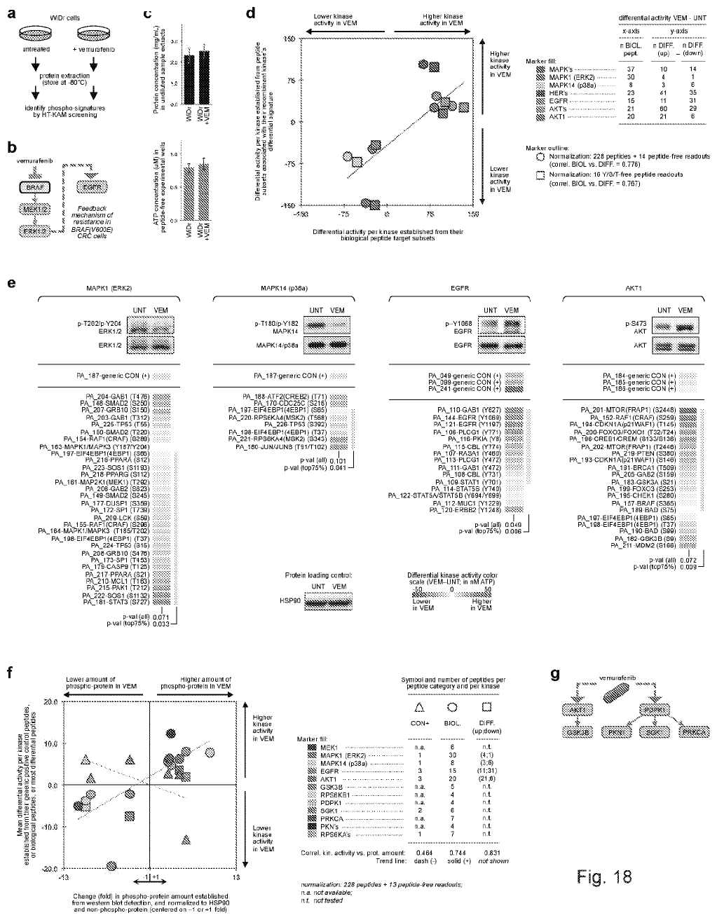

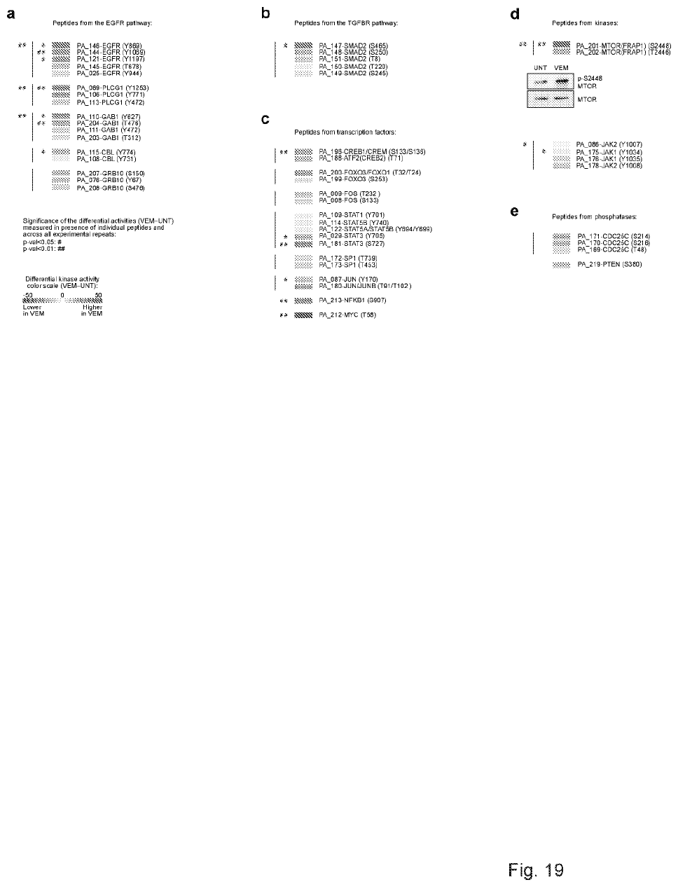

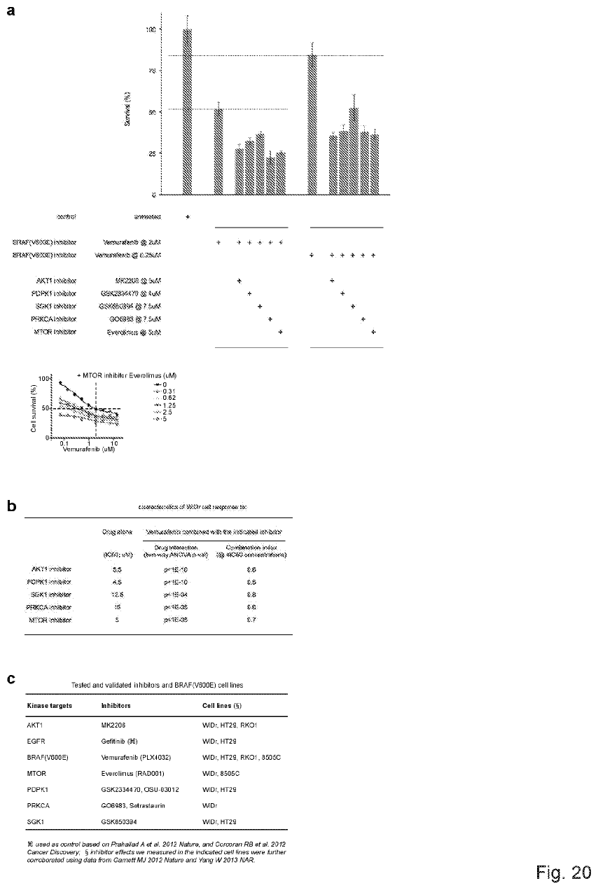

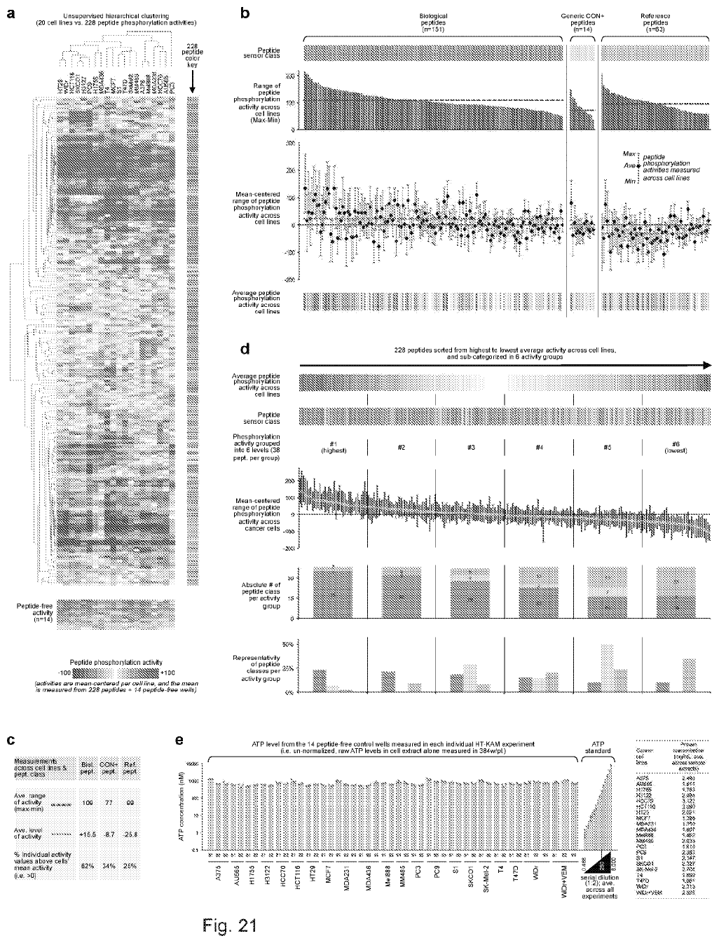

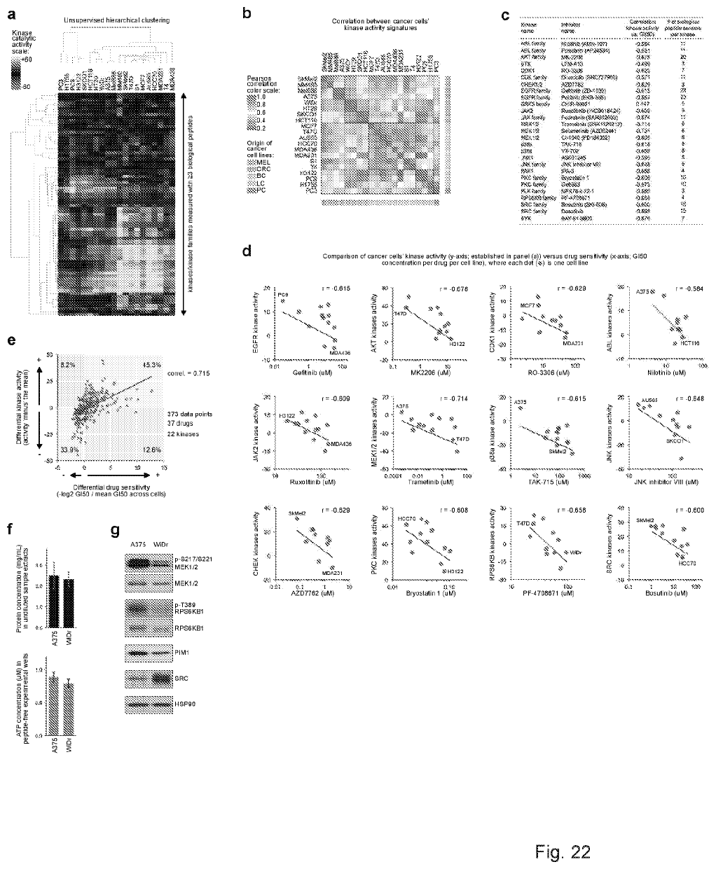

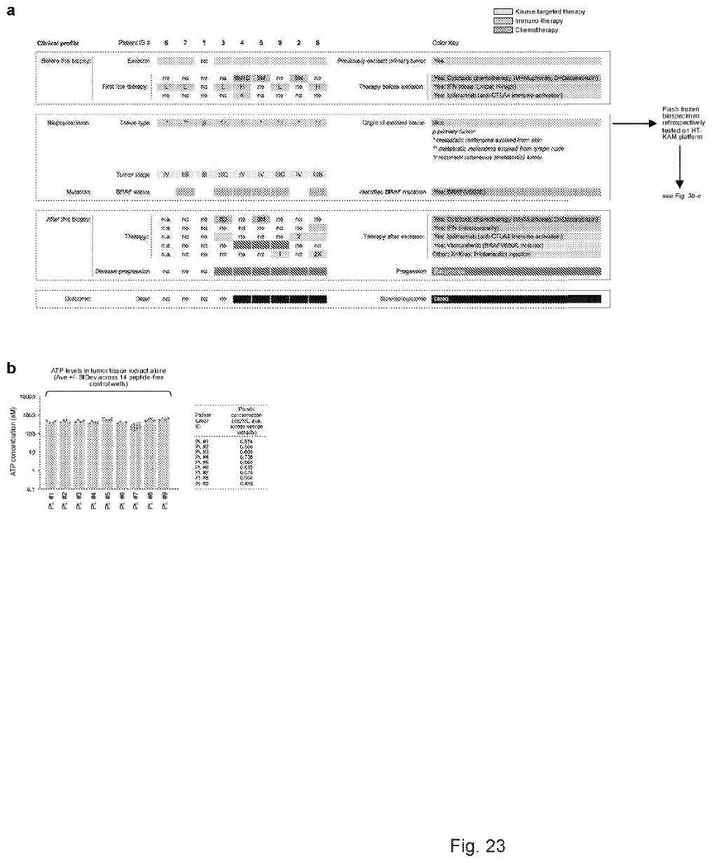

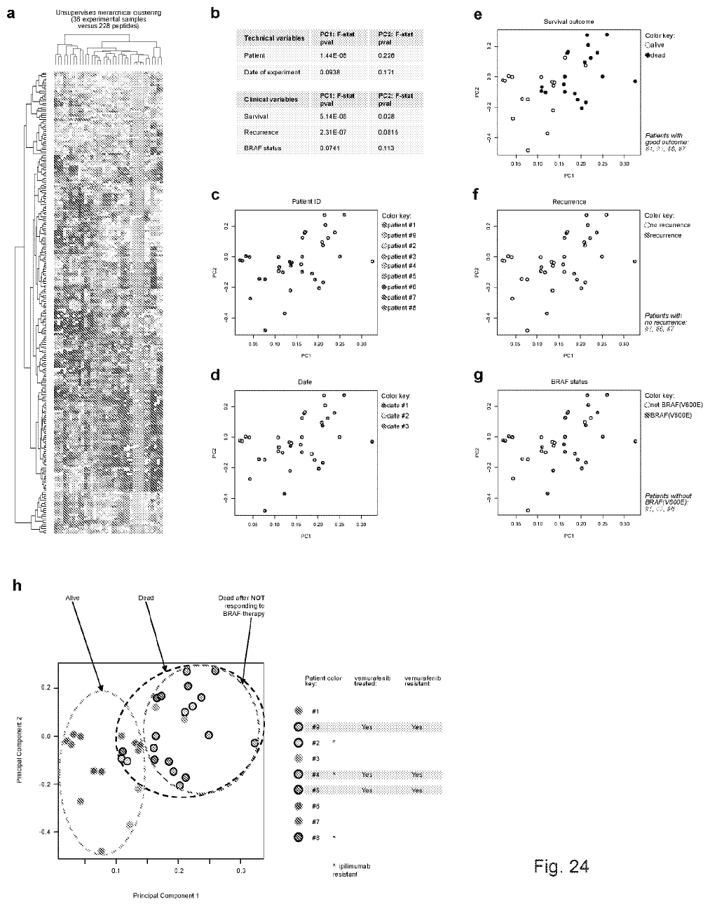

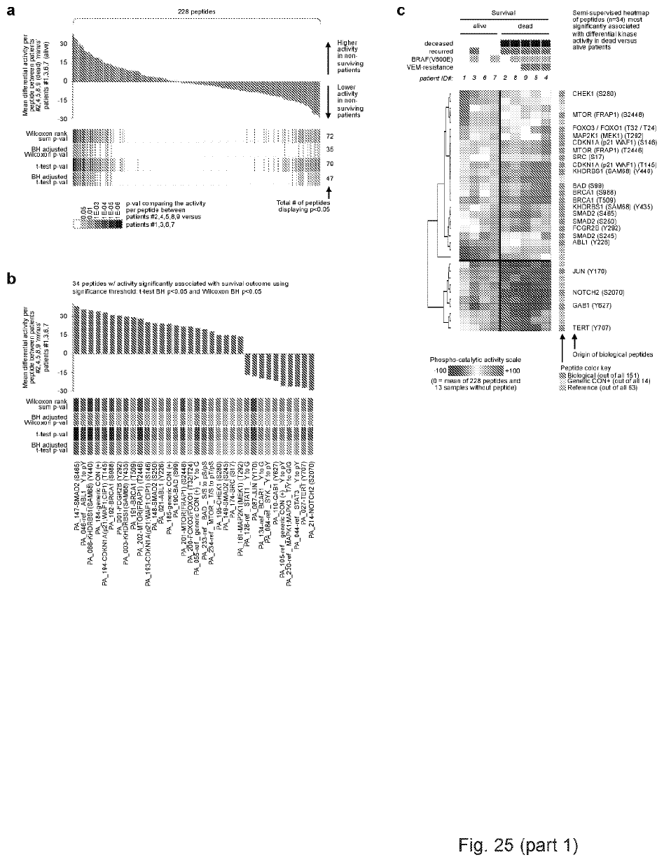

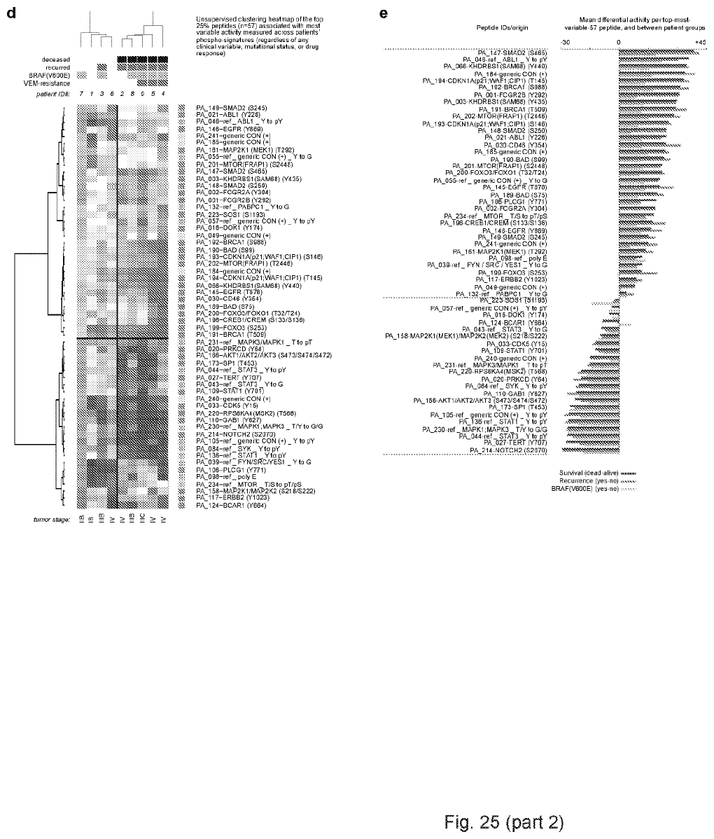

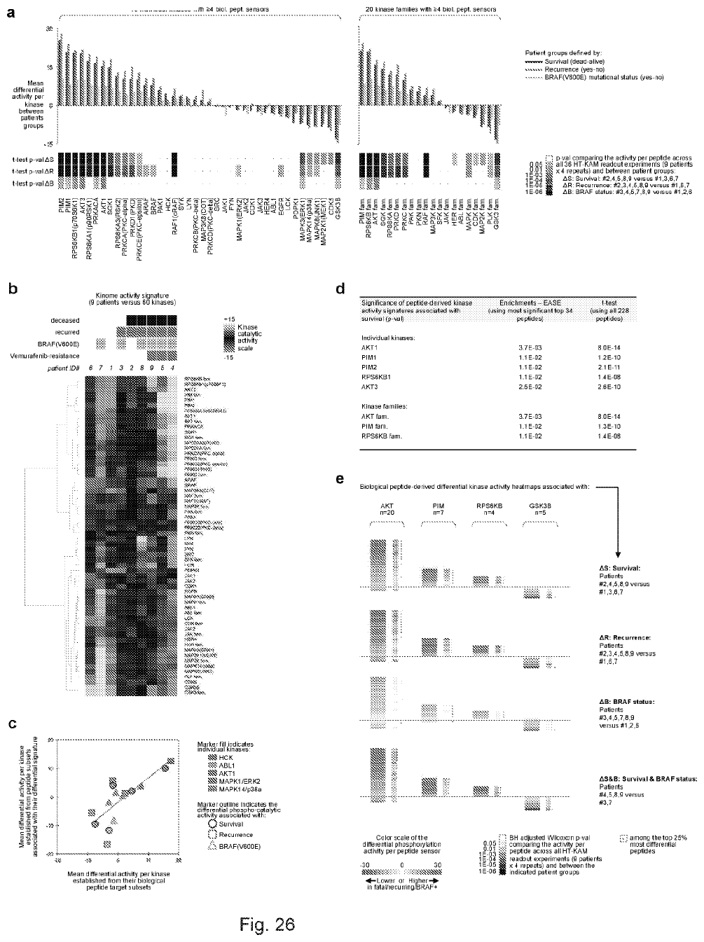

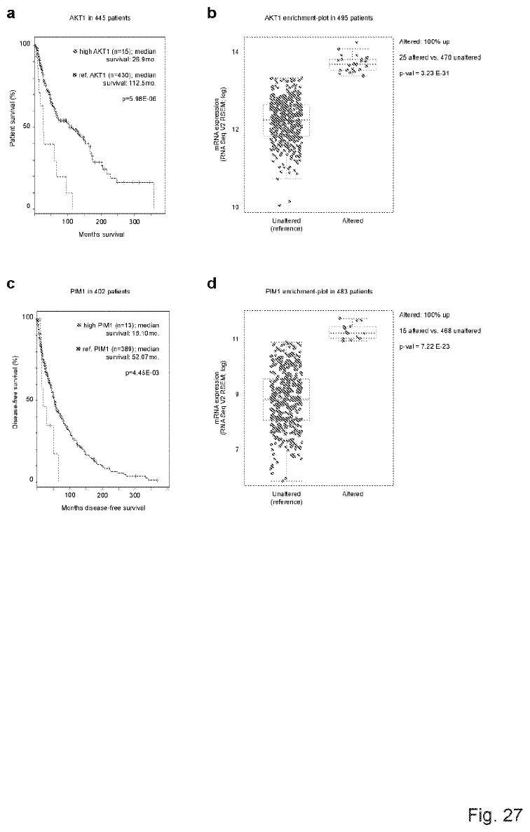

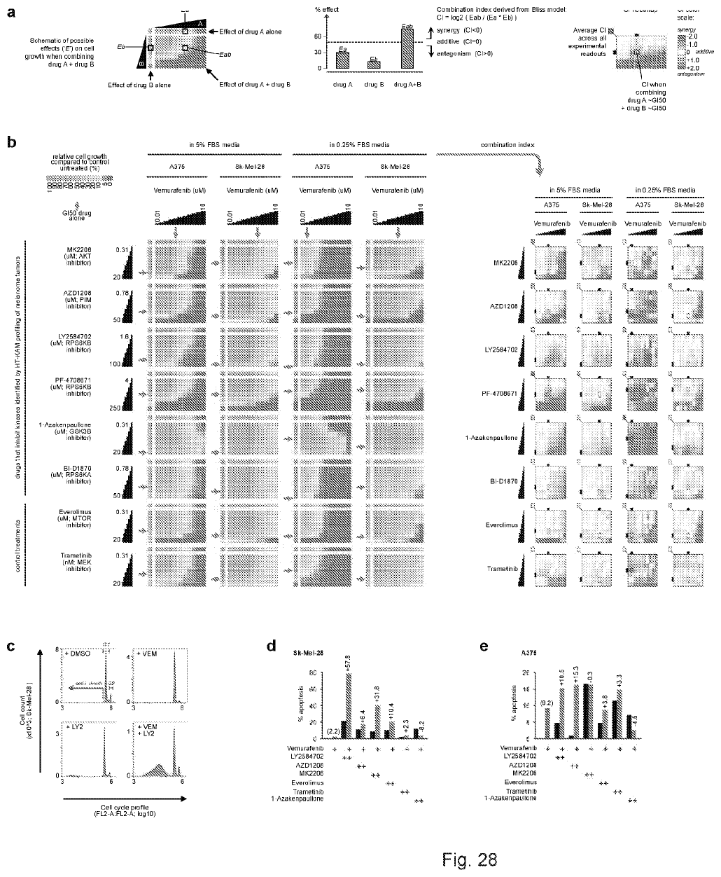

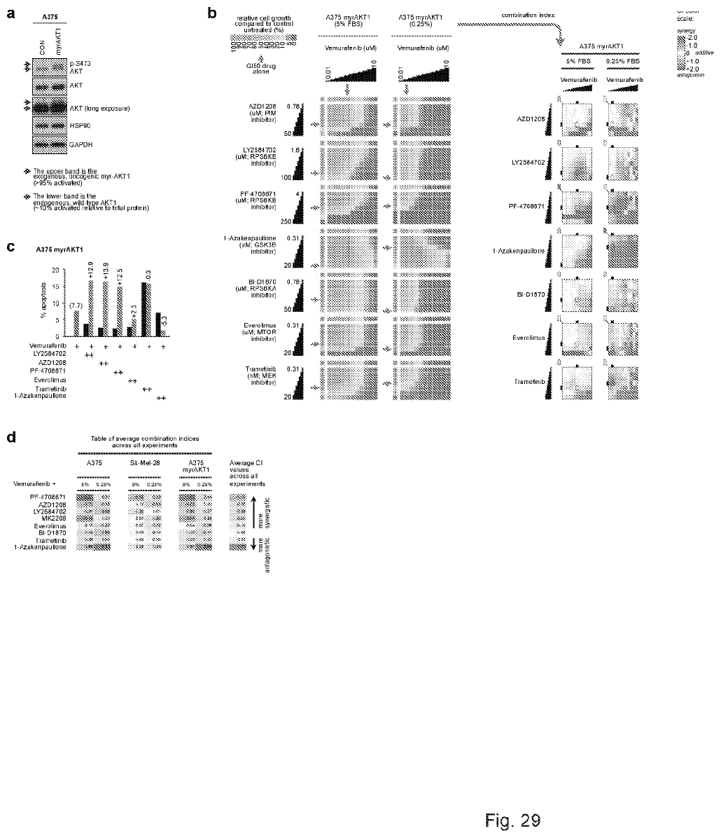

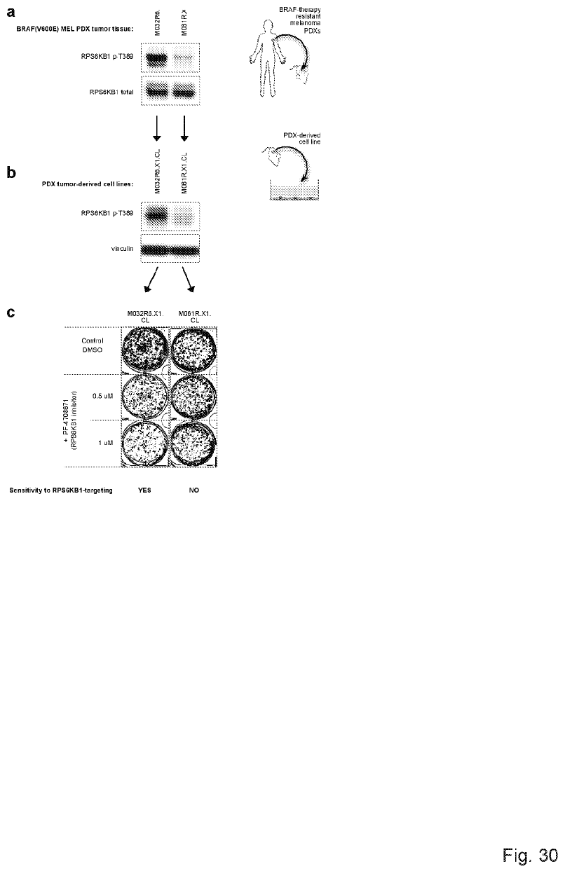

(a) Repeatability of the assay across 120 different 384-well-plate assays. The graph shows a run-to-run comparison of activity profiles for individual kinases. The reproducibility of the assay was high, as measured by Pearson's correlation coefficient for replicate runs of 228-peptide-derived kinase activity profiles of 25 recombinant kinases, and based on >40,000 single ATP-consumption data points.
(b-e) Example of peptide-derived phospho-catalytic signatures for the JAK2 kinase. Four independent experimental runs are compared. ATP consumption profiles derived from luminescence readouts are shown in (b) and compared in (c). Results transformed as internally normalized data by centering ATP consumption profiles against their overall 228-peptide-derived mean, are shown in (d) and compared in (e). The stack bar graph in (d) visually shows that ATP consumption profiles follow similar trends across all different runs: most bars follow the same activity trend of “all-up/higher-than-the-mean”, or “all-down/lower-than-the-mean”, or “close-to-the-experimental-mean”. This relates to the good correlation (>0.9) found between experiments as shown in table (e), and in graph (a).
(f-k) Cumulative bar plots for the other 24 recombinant kinases we tested. Each plot represents the stacked results of activities observed in experimental repeats and in presence of each individual peptide. In each graph, most bars follow the same “all medium” (around ‘0’, i.e. mean kinase activity measured across all 228-peptides per experimental run), or “all-high” (above the mean), or “all-low” (below the mean) activity trends, which further demonstrates run-to-run reproducibility and data consistency. Graph-to-graph comparisons (i.e. kinase-to-kinase) allow discerning kinases' specific catalytic signature.
(l) Cumulative bar plot resulting from merging all profiles of all 25 kinases/120 experiments together. Together, the comparison of panels (d,f-l) show that some peptides are associated with high kinase activity for a particular kinase, or a kinase family, or broadly across multiple kinases/kinase families. Some other peptides are associated with minimal kinase activity (see most negative stacked bars). Peptides from the pool of 63 reference peptides were broadly associated with low kinase activity. As also shown in main FIG. 1b, panels (d,f-m) show that differences in pattern and intensity of activity between members of a same kinase sub-family can be identified (e.g. ABL2 vs. ABL1; AKT2 versus AKT1/3; BRK, FRK or SRMS versus other SFK's; ERK2 vs. p38a). The differential activity of distinct kinase isoforms (LYN A versus LYN B) or ‘wild type’ versus oncogenic kinase (ABL1 versus mutated ABL1^{T315I}) can be found, which functionally corroborates what has been so far inferred from the differential interactomes (PPI) of distinct protein isoforms¹⁶. This shows that kinases affected by alternative-splicing events or ‘minimal’ genetic/oncogenic mutations can exhibit highly specific and distinctive phospho-catalytic functions (as if encoded by unrelated genes). Such results expand the anticipated complexity of cell signaling networks and their alternative cancer-state¹. These data also illustrate the broad dynamic range (over 4 logs of magnitude) of measurable catalytic activities offered by the use of a large variety of peptides, and observable between different kinases. The differential distribution of 228-peptide-derived activities per kinase reveals that each kinase displays distinctive catalytic capabilities. These data show that the HT-KAM strategy is a robust discovery platform that detects and discerns the unique catalytic properties of different kinase enzymes.

FIG. 6. Dilution and time course assays. A subset of kinases and generic positive control peptide sensors commonly used in the field, were interrogated to assess the quality of the results generated with the HT-KAM assay platform.

(a) Activity of kinases measured at different concentrations and over time.
(b) Activity of kinases measured over time in presence of generic positive control peptides versus negative control (non-phosphorylatable) peptides. Significance is shown at the top of the box plot. Kinase-dead recombinant proteins or inactive kinase spliced isoforms (e.g. FYN C) were also used as negative controls to validate results from the assay (data not shown). Profiles in (a-b) match standards in the field, and show the quality of the assay was excellent.

FIG. 7. Comparison of dynamic range to data variation across peptides.

(a) Heatmaps of the top-20 peptides associated with the highest measurable activity per tested recombinant kinase. The first column of each kinase sub-panel indicates three peptide categories: (i) an orange square is for a kinase's generic positive control peptide (as advertised/commonly used); (ii) a yellow square is for any other generic positive control peptide out of the 14 generic positive control peptides included in the HT-KAM assay; (iii) a fuchsia square is for a biological peptide. The second column of each kinase sub-panel shows the peptide-phosphorylation activities for the top-20 peptides (red color scale). Activity profiles were normalized following the method applied in FIG. 1b and FIG. 5, i.e. raw luminescence data were transformed into ATP-consumption data, and then transformed in kinase activity profiles by mean-centering results across 228-peptide sensors per run and per kinase, before averaging profiles for each kinase and comparing significance of peptide phosphorylation profiles across experimental repeats. Activities are shown as a white-to-red scale (saturated red at +100 nM above the mean ATP consumption measured across 228-peptide per kinase). In the third column of each kinase sub-panel, FDR-corrected t-test values per peptide probe are shown to assess the significance of activity profiles. The phosphorylation activities measured with each of the top-20 peptides were compared to the pool of 63-reference peptides included in HT-KAM assays across all experimental repeats for each kinase. FDR-corrected t-test values (BHp) are shown as a white-to-grey-to-black scale (white color cut-off at 0.05; saturated black at 1E-10).
These results indicate that, for many kinases, their best generic positive control peptides have a good, but not necessarily optimal, ability to report on their enzymatic activity. More specifically, the ‘best’ positive control peptide for 17 out of the 25 recombinant kinases did not correspond to one of their originally advertised generic positive control peptides (i.e. yellow surpasses orange for BLK, BRK, FRK, HCK, LCK, LYN A, SRC, YES1, ABL1, ABL1^{T315I}, EGFR, ErbB2, ErbB4, JAK2, CSK, AKT2, MAPK14). Furthermore, for 18 out of 25 kinases, biological peptides were better reporters than their best, originally advertised, generic positive control peptide (i.e. fuchsia surpasses orange for BLK, BRK, FGR, FRK, HCK, LCK, LYN A, SRMS, YES1, ABL1^{T31}, EGFR, JAK2, CSK, AKT1, AKT2, AKT3, MAPK1/ERK2, MAPK14/p38a). In many instances, biological peptides can be considered as at least as good sensors of kinases' activity as currently available generic positive control peptides.
(b) Individual comparisons of FDR-corrected t-test across top activity reporting peptides per peptide category. This graph compares the significance (BH-p values) for the highest phosphorylation activities measured with the top peptide belonging to each of the three categories: (i) best of the advertised/commonly used generic positive control peptide(s) for each kinase (orange dot); (ii) any other best generic positive control peptide (i.e. best of any of the 14 generic peptides other than the ones advertised for the indicated kinase; yellow triangle); (iii) best biological peptide (fuchsia lozenge). Along with data in panel (a), kinase assays show that positive control peptides work well (as expected), but performance can be improved by using/including biological peptide sequences.
(c) Z-factor profiles. Comparing the dynamic range to data variation of ‘positive’ versus ‘negative’ controls is a standard method in the field to evaluate the performance of an enzymatic assay (i.e. Z-factor or Z′). We used this method to assess the quality of individual assays included in HT-KAM experimental sets, and to further assess how using different peptide probes impacts assay readout performance. Z′ was evaluated by comparing ATP consumption values measured in presence of a peptide probe versus in absence of peptide (Z′=1−(3*(StDev Pos+StDev Neg)/|Ave Pos−Ave Neg|)). Z′ values from kinase activity profiles were calculated for the three kinds of peptide probes shown in panels (a-b). Comparing the extent of the assay's dynamic range (˜0.1 to ˜250 nM ATP consumption) to experimental variation (<1 to 20 nM ATP standard deviation) using generic positive control peptides versus no-peptide negative controls (i.e. orange dots; measured across the 14 peptide-free wells), showed overall excellent performance of the HT-KAM assay at the level of individual peptide assays (average Z′=0.53). Yet, Z′ could be significantly increased up to 0.59 when using “any” best peptide out of the 228 peptides included in the HT-KAM screen, and in particular when including biological peptide sensors (fuchsia lozenges). Along with results from activity and significance profiles shown in (a-b), these data show that peptide sequences derived from biological substrate proteins are well suited to measure the phosphorylation activity of kinases.

FIG. 8. Profiles of Area Under the Curve (AUC) values obtained for an increasing number of randomized combinations of peptide sensors to predict the identity of an individual kinase or a kinase family.

(a-c) Progression profiles of AUCs for individual kinases and kinase families. In the analysis described below, the ‘outcome’ we wanted to predict was the identity of a kinase based on peptide activity profiles. For example, for AKT, the analysis outcome was “is this kinase in the AKT family: yes or no?” when using the activity profile from one or multiple peptides where peptide(s) were ‘drawn’ from any of the 228 peptides, and activities compared across all 25 recombinant kinases and all experimental repeats. To do so, Diagonal Linear Discriminant Analysis (DLDA) class predictors were built for each kinase or kinase family using randomized combinations of an increasing number of peptides to discriminate one given kinase (or kinase family) from others. The performance of the DLDA classifier was defined as the AUC for predicting the identity of a kinase (or kinase family) from repeated iteration (i=1,000) of random peptide sampling. The confidence intervals of AUC's were computed using the DeLong method (from the pROC package of R). AUC values (y-axis) obtained for any number of combined peptides (x-axis) are shown as progression profiles of AUC's (box plots) for individual kinases (a-b) and kinase families (c). Plots show the effect of increasing n on AUC values, where n random peptides are drawn out of 228 peptides, and where n spans from 1 to a combination of 50 peptides (graphs in panels (a,c)) or from 1 to a combination of 100 peptides (graph in (b)). We analyzed individual kinases for which enough experimental repeats were executed (here we chose a stringent cut-off of ≥6 repeats; i.e. ABL1, AKT1, EGFR, MAPK1/ERK2, MAPK14/p38a, HCK, SRC; panels (a-b)), and for kinases grouped by subfamilies (with sufficient (≥6) repetitions per family; i.e. ABL, AKT, HER, MAPK, SFK; panel (c)). For each of the 50 boxplots per graph shown in (a,c), or for each of the 100 boxplots in the graph shown in (b), the line in the middle of each ‘box’ is the median of the data, the length of the box is 25% and 75% of the data, and the whisker is (25%−1.5×(InterQuartile Range)) (IQR) and (75%+1.5×IQR). The outliers (points) are anything that falls outside of that range.

Results in (a-c) show that, for every kinase or kinase family, the sensitivity and specificity of a kinase-assay intended to measure their phospho-catalytic activity, is systematically improved when: (i) including an increasing number of combined peptide sensors; and/or (ii) when using particular subsets of peptides that perform significantly better than other combinations within a particular iterative number of peptides (i.e. higher AUC's within a given n-number of peptides). These results also show that some unique combinations of few peptides can behave as highly specific and sensitive reporters of a given kinase (i.e. these peptide subsets would be located toward the top left corner of each plot; note that the results of this method/analysis do not—however—define which peptide sets differentiate with the highest probability and highest specificity/sensitivity between different kinases, which is a question we resolve later in FIGS. 10, 11, 14, 15 and shows that biological peptide subsets happen to be exquisitely well-suited for). It can be noted from these data that, in the perspective of expanding the coverage of the HT-KAM platform to monitor more/other kinases, the computational analyses and modeled data in (a-c) already demonstrate that including additional peptide probes would only increase the capability of the HT-KAM system to accurately predict the identity of more/different kinases out of their phospho-catalytic activity signatures. Together, these results demonstrate that our strategy of including a multiplicity of peptide sensors considerably improves the sensitivity and specificity of any kinase assay.

FIG. 9. Kinase phosphorylation activity measured in presence of individual generic positive control peptides. To further show that including a multiplicity of peptide sensors improves the performance of a kinase assay, we asked whether any individual generic positive control peptide could provide the specificity needed to accurately identify individual kinases.

(a) List of kinases whose activities are ‘expected’ to be measured with the positive control peptides included in the HT-KAM assay. The 14 generic peptides included in the 228-peptide library are commonly used as reporters of the phospho-catalytic activity of >63 individual kinases and >27 kinase families (e.g. ABL, AKT, ERK, HER, p38, SFK, TK, etc).
(b) Comparison of kinases' phosphorylation activity patterns across positive control peptides based on either what is anticipated from literature (top panel), or experimentally measured (bottom panel). In the top panel, the red-colored areas correspond to peptides expected to report on the activity of the indicated kinases, whereas the blue areas are not advertised as such. In the bottom panel, the blue-to-white-to-red heatmap corresponds to the experimental profile of recombinant kinases' activity levels measured in presence of these peptides. The bottom panel is an excerpt from the complete 228-peptide phospho-catalytic activity signature of kinases. In principle, the red areas from the top panel should match the ‘redder’ areas from the bottom panel, and, as critical, the blue areas from the top panel should match the ‘bluer’ areas from the bottom panel.
(c) Detailed comparison of kinases' activity level for each generic positive control peptide. The 14 individual graphs display the activity of all 25 recombinant kinases for each of the 14 peptides. In each graph, the red bars indicate catalytic activities expected to be the highest/most positive. Conversely, black bars are not anticipated to display high activity or any activity.
(d) Concordance between expected and experimental activities. The % concordance was evaluated based on whether experimental readouts belong or not to their expected activity groups (vertically: per kinase; horizontally: per peptide). The overall average concordance is ˜52%. Cross-reactivity between kinases/peptides observed in (b-d) indicates that it would not be possible to use any individual positive control peptide as a mean to specifically identify or differentiate a particular kinase from other kinases.
(e) Comparison of individual peptides' AUC to evaluate how specific and sensitive each of the 14 positive control peptides is to identify their respective kinases. AUCs were calculated from repeated iteration of single peptide sampling for kinases tested ≥6 times (i.e. HCK, SRC, ABL1, EGFR, AKT1, MAPK1/ERK2, MAPK14/p38a). This analysis compared all 228-peptide phospho-catalytic profiles across all 25 recombinant kinases and all experimental repeats (method described in FIG. 8). This method answers the question: “how good is an individual peptide at predicting the identity of a kinase?”, and results can be interpreted as: “the higher a peptide's AUC is for a particular kinase, the more this peptide (and this particular level of phosphorylation for this peptide) is good at discriminating this kinase from all other kinases”. The top section shows all calculated AUCs for all 14 positive control peptides across all interrogated kinases (color-coded scale of AUCs shown on the right; kinase name on top). A black side-bare next to AUC(s) designates positive control peptide(s) expected to report on the indicated kinase. To help compare the performance of individual positive control peptides' specificity and sensitivity, the bottom section displays the AUCs for 14 individual biological peptides with the highest AUCs for each kinase. Results show that most individual positive control peptides do not provide the highest possible specificity and sensitivity for the kinases they are expected to report on. Furthermore, most individual positive control peptides are outperformed by individual biological peptides. These data provide new leads on how to improve single-peptide kinase assays using (biological) peptides found with the HT-KAM strategy. More critical to the concept underlying our study, these results underline why a multi-peptide approach is a valuable alternative to single peptide-based activity measurements when investigators want to specifically and differentially identify a given kinase from other kinases.

FIG. 10. Systematic identification of combinatorial peptide sets that best differentiate between kinase families. We implemented computational methods to define which unique set of peptides most significantly distinguishes a kinase from all other kinases. (a-e) Differential phospho-catalytic signatures of kinase families. The phospho-catalytic activity heatmaps of ABL (a), AKT (b), HER (c), MAPK (d), SFK (e) are established from the peptide subsets that best differentiate each kinase family from all other kinases. Peptide-IDs are listed on the right side of each heatmap. A scale of catalytic intensity is shown underneath (blue-to-white-to-red of mean-centered activities). The two color-coded columns on the left of each heatmap show the type/origin of peptides, and the mean difference in activity: (i) peptide types or origin are shown in red shade (biological), yellow shade (generic positive control), grey (reference); (ii) the mean difference in activity is shown on the left, as an indicator of how significantly more (pink) or less (green) active a kinase family is in presence of the listed peptide that was included in their differential signature.

The first step of the computational method was to systematically compare each of the 228-peptide activity signatures from all kinases belonging to a given family with ≥6 experimental repeats (i.e. ABL, AKT, HER, MAPK, SFK), to the 228-peptide signatures of all other kinases belonging to all other families, and then select peptides associated with the most differential activities based on whether any of the 228 peptide-associated activity values passed or not a significance threshold of p<0.05 for both FDR-corrected t-test and Wilcoxon rank sum test. This implies that: (i) the selected, most differential peptides can be associated with either low, or high, or ‘average’ phospho-catalytic activities specific to a kinase family if it significantly contrasts with activities observed across all other kinases (following this principle, a peptide can be found as part of the differential signature of multiple kinase families owing activity levels and significances that are specific to the differential signature of its given kinase family versus all other kinases); (ii) the activities from the selected, most significantly differential peptides specific to a kinase family follow a trend that may vary from one individual kinase to another within that family (and between experimental read outs), and some individual kinases may also cluster away from the majority of other kinase family members (which underlines the functional precision of the combinatorial measurements provided by the HT-KAM assay toward the systematic identification of the specific functional attributes unique to a kinase family yet can distinguish sub-family members).

The second step of this analysis, was to extract all phospho-activity values that match the significantly differential peptides out of the 228-peptide-derived activity values for each individual family, and then apply unsupervised hierarchical clustering to group peptides and kinases based on their functional relationship (classification trees at the top and far left of each heatmap).

Each panel (a-e) also includes a graph on the top right that shows the Receiver Operating Characteristic (ROC) curve and AUC value calculated for the specific subset of most differential peptides for each kinase family. The method used to assess the performance of each peptide set (i.e. its sensitivity and specificity) was the same as the one used to predict the performance of increasing numbers of random peptide sets described in FIG. 8. We used the 228-peptide/25 kinases/120 experiments dataset to build classifiers, and then predict and compare performance using the exact differential peptide set associated with each kinase.

As an example, panel (b) shows that a unique combination of 89 peptides out of 228 peptide sensors best differentiates AKT-family kinases from all other kinases/kinase families. This 89-peptide subset includes both significantly high, low and intermediate activity features of AKT that uniquely differentiate AKT from all other tested kinases. This unique set of 89 peptides is capable of detecting/identifying AKT with a high degree of sensitivity and specificity (i.e. AUC=0.945). AKT's differential signature includes every type of peptides, whether biological, generic or reference, and whether phosphorylated or not by AKT. We complemented our analyses using Monte Carlo cross validation to further estimate how accurately our predictive model performed, which confirmed the validity of the differential peptide signature (data not shown).

Results in (a-e) show that differential peptide phosphorylation signatures can be assigned to every kinase families. This also demonstrates that using a multi-peptide activity-screening assay such as the HT-KAM strategy can find specific combinations of peptides that can serve as predictors of kinases' identity. As displayed in (a-e) and summarized in FIG. 1e, these results also indicate that the majority of peptides most capable of discerning kinases' unique phospho-catalytic activities are biological peptides.

FIG. 11. Peptide sets that best differentiate individual kinases. We applied the computational methods designed in FIG. 10 to identify the subsets of peptides that most significantly distinguish an individual kinase from other kinases.

(a-e) Differential phospho-catalytic signatures of kinases. Differential signatures and AUCs were calculated for individual kinases with ≥6 experimental repeats: ABL1 (a), AKT1 (b), MAPK1/ERK1 (c), MAPK14/p38a (d), HCK (e). We also identified the differential activity signatures of EGFR and SRC kinases, which respectively included 42 and 43 peptide features associated with AUC's of 0.840 and 0.893, and that successfully passed the significance threshold (p<0.05) for either FDR-corrected t-test or Wilcoxon rank sum test, but since they did not concurrently pass both criteria, we decided not to include their profiles here.
(f) Bar plot summarizing the number and origin of peptides composing each kinase-specific differential signature. Biological peptides constitute the majority of peptide sensors making the differential catalytic signatures of individual kinases.

Together, results from FIGS. 8-11 and FIG. 1b-e show that using an array of peptides to measure the activity of kinases is an effective approach to functionally distinguish and identify different kinases. Such multi-peptide sensor-based kinase identification system is superior to any single probe enzymatic assay, including assays relying on a generic positive control peptide or any individual peptide in general. The level of differentiability, sensitivity and specificity offered by our multi-peptide platform approach is such that all kinases/kinase families can be predictably identified based on their activity signatures. Even though some of the kinases we chose to examine can be considered as more difficult to distinguish since they are biologically closely related (e.g. SFKs or ABLs), our system was capable of identifying and differentiating them (a functional distinction that no individual peptide was able to predictably achieve). As well, the signatures of kinases of more distant biological functions/unrelated genetic families were more distinct from other kinases and systematically distinguishable (e.g. MAPKs or HERs). In fact, our computational analyses and experiential data demonstrate that if we were to test larger numbers of recombinant kinases belonging to more distant kinase families, their activities would be more divergent from other kinases, and thus easier to measure/identify. While these results imply that the HT-KAM strategy could be used as a discovery tool to identify peptide sensors most uniquely differential and predictive of the identity of a kinase/kinase family, these results also indicate that the biological peptides of kinases are an already available subsets of specific/sensitive/differential sensors that are well-suited to decipher between kinases and their respective levels of activity.

FIG. 12. Phospho-catalytic signatures of kinases established from their respective biological peptides. The computational analyses developed in FIGS. 10-11 and FIG. 1e show that biological peptides contribute most to the differential peptide signatures of kinases, so we asked whether kinases phosphorylate their respective biological peptide sequences.

(a-g) Activity profiles of recombinant kinases measured in presence of their respective biological peptides. Individual kinases are organized by kinase sub-families: AKTs (a), MAPKs (b), SFKs (c), ABLs (d), HERs (e), and include JAK2 (f) and CSK (g). The origin of peptides is indicated on the left of each panel. The phosphorylation activity per indicated biological peptide is averaged from ‘n’ independent experiments. Kinase activity is color-coded blue-to-white-to-red from low-to-medium-to-high activity (i.e. a white/dim color indicates measurable enzymatic activity within the spectrum of low-to-high activity). For each kinase heatmap, activities are organized by decreasing intensity from top/high to bottom/low. The average catalytic activities obtained from three different peptide groups (random, Y/S/T-free, reference) are shown for comparison at the bottom of each panel. The t-test p-values comparing the activity profiles of each biological peptide to random or Y/S/T-free or reference peptide groups across all experimental runs for a given kinase (indicated by ‘n’ at the top of each kinase-specific panel), are represented as a grey scale.
(h) Box plots of kinases' activity measured in presence of either their biological peptide subset, or 5-random, or 16-Y/S/T-free, or 63-reference peptides. Each box plot distribution of data displays: minimum, first quartile, median, third quartile, and maximum. Each box plot includes all experimental values across all repeats. Normalization and analyses of profiles follow previously described steps. The number of biological peptides per kinase is indicated underneath each box plot in the top graph. The t-test p-values comparing the overall activity profiles established from biological peptide versus either of the three other peptide groups measured across all kinases, are indicated in brackets on the right side of the graphs (respectively p<2E-100, p<2E-110, p<6E-121).
(i) Table of significance comparing kinases' phosphorylation activity profile established from their biological peptide subsets versus either of the other ‘control’ peptide groups (i.e. random, or Y/S/T-free, or reference).

Results in (a-i) show that biological peptide probes established from substrate protein regions of kinases were overall associated with measurably and significantly higher levels of phosphorylation activities than ‘control’ peptides. More than 94% of all kinase activities measured in presence of their biological peptides were greater than activities measured in presence of the 5-random peptide set. Differences in kinase activity between biological and reference peptide sets were highly significant (p<7E-11 for AKT1, AKT3, MAPK1/ERK2, FYN A, HCK, LYN A, ABL1, and JAK2; p<4E-2 for AKT2, MAPK14/p38a, BLK, BRK, LCK, LYN B, SRC, HER2, HER4). Individual kinase activity values derived from their individual biological peptides were significantly different from the reference peptide set (27.2% with p≤0.01 and 66.7% with p≤0.05). Kinase activity levels were also significantly higher when measured in presence of their ‘non-modified’ biological peptides than with their mutated or pre-phosphorylated counterparts included in the assay (see FIG. 13 for details). Finally, phosphorylation activities measured with biological peptides of nearly identical sequences were similar (e.g. MTOR T2446 and MTOR S2448; CDKN1A T145 and CDKN1A S146; RAF1 Y340 and RAF1 Y341; JAK2 Y1007 and JAK2 Y1008; GSK3B S9 and GSK3A S21), which further demonstrates the repeatability of the kinase activity measurements made with their biological peptides.

(j) Unsupervised hierarchical clustering of kinases' functional signatures using only biological peptides. The clustering of kinases (top) demonstrates that the 151 biological peptides included in the assay are excellent functional discriminators of kinases' catalytic signatures. Kinases belonging to a same sub-family most closely resemble each other, while most functionally distant kinases segregate away from each other.
Together, results in (a-j) demonstrate that biological peptides are effective combinatorial sensors to functionally differentiate kinases from each other, and to measure the enzymatic activity of their respective kinases. Kinases are significantly and specifically more capable of phosphorylating a vast majority of their predicted biological peptide targets than reference peptide sets.

FIG. 13. Comparing the activity of kinases measured in presence of their biological peptides versus modified peptide counterparts. To further assert the preference of kinases to phosphorylate their biological peptide targets, we compared how significantly different the levels of phosphorylation activity of kinases were when measured in presence of their biological targets versus in presence of the mutated or pre-phosphorylated biological peptide counterparts.

(a) Difference in the activity of kinases comparing pairs of non-modified versus modified biological peptide sequences. In the waterfall plot, each teal bar shows the difference in activity between a biological peptide and its mutated (Y/S/T→G) counterpart. Each violet bar shows the difference in activity between a biological peptide and its pre-phosphorylated (Y/S/T→pY/pS/pT) counterpart. Each peptide pair substrate's origin and target site along with its kinase is indicated underneath the graph. Results are derived from all experimental measures for all kinase. The significance of the difference in activity in presence of two different peptides was assessed in pairwise comparisons between experimental runs (grey scale of t-test values underneath the graph).
(b-c) Overall comparison of activity profiles of Tyrosine kinases or Serine/Threonine kinases measured in presence of their predicted Y/S/T-containing biological peptides versus any Y/S/T-free biological or reference peptide. The phosphorylation activities of both Tyrosine kinases and Serine/Threonine kinases are significantly higher in presence of their Y/S/T-containing biological peptides than any Y/S/T-free biological or reference peptides.

Along with results in FIG. 12, data in (a-c) indicate that the variety of biological peptides and control peptides included in our assay provides a practical framework to systematically validate and compare activity profiles within individual assays, or across technical repeats, or between different samples. These analyses show that kinases significantly prefer to phosphorylate their ‘un-modified’ biological peptide targets.

FIG. 14. Using HT-KAM as a ‘discovery platform’ to identify best targets of kinases, finds that biological peptides are systematically included among the most significantly high activity profiles of kinases.

(a) Binary heatmap showing peptides associated with high (red) or low (blue) activity for each kinase. For this analysis, each kinase was tested independently of all others, and we used two separate computational approaches to compare levels of ATP consumption per individual peptide to the pool of 63-reference peptides.

In the first approach, the average 228-activity data points from all experimental repeats was used in a Kalmagorov-Smirnov (KS) test comparing each 165-non-reference peptides (i.e. 151 biological peptides, and 14 positive control peptides) to the 63-reference peptides (p values with or without BH correction controlling for false-discovery rate). In parallel, the mean and standard deviation (SD) of the 63-reference peptides was computed to then identify which peptides among the 165-non-reference peptides display activity signals >2 fold SD from the mean (>highest 2.5% of reference).

In the second approach, all experimental replicates (instead of averaging them as in the first method) were used in either a linear additive model with BH corrected p-values from each 165-non-reference peptide versus 63-reference peptide (BH.p.lam<0.05 threshold), or an ANOVA model with BH corrected replicate error.

The overlapping results of these two approaches and their statistical cut-offs identify the most significantly and stringently selected high—and low—activities per peptide per kinase. This highly conservative method finds that 110 biological peptides act as best probes/sensors of kinases' individual phospho-catalytic activities. Binomial high/low activity levels are represented as a heatmap in this panel (a; high in red, low in blue), along with the 25-tested recombinant kinases organized by subfamily (indicated at the top), and the identified 124 peptides organized based on unsupervised clustering (correlation tree on the left; peptides probe ID's/origin on the right).

(b) Analysis of activity profiles established in presence of inhibitors to validate results found in panel (a). Here, the underlying postulate is that, when the activity of a kinase is measured in presence of an inhibitor, any peptide associated with a significant decrease in activity may be considered as a suitable probe to detect the activity of this kinase. Such data would also retrospectively validate (or not) the previously identified peptides from the method in (a). In the three graphs shown in (b), the model system uses ABL1 or LYN A kinases, and serial dilutions of imatinib or dasatinib.

For instance, quadrant A shows a strong correlation between levels of inhibition (captured by the Pearson correlation coefficient between imatinib concentration and ATP consumption for each peptide; y-axis), and the activity level per peptide in an untreated context (x-axis) (R² (Fisher(inhibition), activity)=−0.48; p=2.75e-14). Importantly, the relationship between the activity levels of (all 228) peptides incubated with ABL1 in an untreated setting and their level of inhibition under increasing concentrations of imatinib, also showed that the peptides that report higher activity levels of ABL1 kinase (i.e. dots located toward the right end of the x-axis; indicating highest ATP consumption), exhibit greater inhibition of that activity in presence of increasing concentrations of imatinib (i.e. dots located toward the bottom end of the y-axis; indicating strongest negative correlation and thus strongest inhibition). These peptides (bottom right area) largely overlap with the peptides found with the previous method in (a) (indicated as red dots in quadrant A). For instance, biological peptides JUN Y170, CDK5 Y15, WASL Y256, or BTK Y223 are at the top of the list of peptides most significantly associated with high ABL1 activity, and they also show strong inhibition of activity in presence of imatinib (shown as red dots in (b)), which confirms that they are ABL1 substrates targetable by anti-ABL1 therapeutic drug. So, these data are a finding consistent with the initial validation purpose of the analysis. Importantly, this method also finds that the biological peptide targets of ABL1 are most systematically and significantly associated with the measurable response of ABL1 to ABL1-inhibitors (besides the 4 peptides previously mentioned, this method also finds JAK2 Y1007, MAP4K1 Y232, ABL1 Y226, TP73 Y99, CDKN1B/p27 Y88, MDM2 Y394, RAD51 Y54 as good reporters of ABL1's activity; shown in main FIG. 1j).

Similar results were obtained for dasatinib-treated ABL1 (quadrant B) and dasatinib-treated LYN A (quadrant C) using the same experimental validation approach and statistical analysis.

(c-d) Comparison of the most repeatable peptide-derived activities of ABL1 to the spectrum of activities established from the differential signature of ABL1. Following the x-axis of the graph shown in (c), the 22 peptides that behave as the most internally robust sensors of ABL1 activity are represented as 14 green squares+8 black triangles (data derived from the analysis used to generate panel (a)). Following the y-axis of this graph, the 40 peptides that best differentiate ABL1 activity signature from all other kinases' signatures are represented as 32 purple lozenges+8 black triangles (data established in FIG. 11a). The dashed rectangles (i) and (ii) outlined in graph (c) match the dashed areas (i) and (ii) in panel (d), where peptides' IDs/origins are listed. The red-filled or red-highlighted-margin squares or lozenges or triangles or circles in (c) correspond to biological peptide targets of ABL1 as defined by PhosphoAtlas and included in the HT-KAM peptide library. Noticeably, all ABL1's biological peptide targets are located on the top right corner of the graph (i.e. reporting on high ABL1 activity). This analysis also finds that peptides associated with significantly low/minimal ABL1 activity correspond to biological targets of Ser/Thr-kinases (dashed rectangle (ii) in the lower left area in (c) and listed in panel (d) as biological peptides established from phospho-target sites: AKT1 T308, MAP2K1 T192, SOS1 S1193, BRCA1 S988, NFkB1 S907, SMAD2 T8, CREB1 S133, EGFR T678). These observations directly support why combinations of ‘disparate’ biological peptides are intrinsically good discriminators and identifiers of different kinases.
(e-f) Venn diagrams intersecting results from analysis in (a) (i.e. robustness of peptide-phosphorylation activities) and analysis in FIG. 11 (i.e. most differential peptide activities). Results similar to ABL1 data (e) were found for other kinases, such as AKT1 (f), or SFK's, HER's, MAPK's (data not shown). In all cases, biological peptides act as robust sensors of the differential and individual phospho-catalytic activity signatures of kinases.

Based on the computational methods we developed and peptide library we designed to showcase our system, we find that biological peptides are systematically associated with most significantly and measurably high activity profiles of kinases.

FIG. 15. Comparison of the specificity and sensitivity of kinases' signatures established with their biological peptide subsets, or their generic positive control peptide sets, or random peptide sequences.

(a-b) ROC curves and AUC profiles of kinase families and individual kinases. The ROC curves and AUC values of each kinase measured with their specific subsets of either all-biological (red), or all-positive (gold), or all-random (grey) peptides are shown as lines and annotated within each graph. ROC curves and AUC values were established using the method explained in FIG. 8.
(c) Comparison of AUCs measured across kinases and measured from all-biological (red) vs. all-positive (gold) vs. all-random (grey) vs. differential (violet) peptide sets. Averages, standard deviations and p-val are calculated from AUC's values shown in (a,b) for biological (red), positive (gold) and random peptides (grey), and from AUC's values for the differential peptide subsets (violet) that were analyzed in FIGS. 10-11.

Results from these plots show that the combinations of biological peptides are very good predictor of kinases' identity. Specifically, for kinase families ABL, AKT, HER, and MAPK, and for individual kinases AKT1, EGFR, MAPK1/ERK2, HCK, SRC, the AUC values obtained from their biological peptides are excellent (on average >0.94). It can be noted that the somewhat lower AUC observed for SFK's biological peptides (i.e. 0.859) was attributable to peculiar activity differences from individual SRC family members. Indeed, the AUC derived from the predicted biological peptides of SFK's was increased when the most dissimilar members of the SFK family and their related peptides were ‘excluded’ (AUC augments from 0.859 to >0.92, while singling out FGR and FRK kinases along with SRMS and BRK as majorly distinctive functional subclasses among SRC kinases, and in fact display little overlap among subsets of biological peptide target pools, hence the differential effect on specificity and sensitivity of SFK's catalytic signatures; analysis/data not shown). Following such principle, entire predictions on the functional distance between kinase enzymes (instead of kinase genes) could be made, and could reshape kinase classifications. This concept also relates to the good-yet-somewhat-lower AUC found for the MAPK family, since the two recombinant MAPK's (ERK2 vs. p38a) we studied are functionally/biologically quite distant from each other, and thus their enzymatic activities for their respective biological peptides are fairly dissimilar (which was already noticeable in earlier analyses/figures).

In addition, it can be noted from panel (c) that the specificity/sensitivity (AUC's) from biological peptide subsets performed almost to the same degree as the differential peptide sets computationally identified in FIGS. 10-11 (e.g. ABL AUC(biological peptide set; n=11)=0.974 vs. ABL AUC(differential peptide set; n=34)=0.911; AKT1 AUC(biological peptide set; n=20)=0.986 vs. AKT1 AUC(differential peptide set; n=27)=0.977; HER AUC(biological peptide set; n=22)=0.967 vs. HER AUC(differential peptide set; n=76)=0.948)). AUC's from positive control peptide subsets performed systematically and significantly less well.

These results demonstrate that biological peptide subsets are well-suited predictive sensors to specifically, sensitively, and differentially detect their respective kinases. These data underline the strict precision of the HT-KAM assay/data analysis system we developed, and its ability to accurately account for peculiar functional differences between kinases, including between individual kinases otherwise considered as genetically related.

FIG. 16. Measuring the effects of kinase inhibitors using biological peptides. Measuring the effects of inhibitors on kinases' activity is a good way to evaluate the performance of biological peptides as sensors of kinases. So, we tested whether biological peptides of kinases reported on the inhibited activity of their kinases in presence of drugs at multiple concentrations.

(a) Table of IC50 concentrations of imatinib, dasatinib, and staurosporine for ABL1, ABL1^{T315I}, LYN A and AKT1 kinases. IC50 values are indicative averages established from IC50 estimations measured in biochemical assays reported in the literature.
(b) Monitoring the effects of kinase inhibitors on the activity of ABL1, ABL1^{T315I}, LYN A and AKT1 kinases using their respective subsets of biological peptides as sensors of changes in activity. Experimental concentrations of imatinib and dasatinib correspond to 0.01×, 1× and 100×IC50 concentrations specific to ABL1 (i.e. 0.002 uM, 0.2 uM, 20 uM, and 0.01 nM, 1 nM, 100 nM, respectively). These concentrations are thus anticipated to affect the other recombinant kinases tested here (ABL1^{T315I}, LYN A and AKT1) with less or little-to-no effect on their levels of activity. This means that the relative differences in reported IC50 concentrations between ABL1 and LYN A for imatinib (0.2 versus 100 uM) and dasatinib (1 versus 9 nM) can be used to compare the measurable differences in effects of inhibitors. As well, AKT1 can be used as a ‘negative control’ for lack of response. Every experimental phospho-signature was initially measured across all-228 peptides, and either in presence or absence of inhibitor (i.e. each sample profile was normalized against the mean activity level measured across 228 peptides, and then averaged across all 3 independent replicates of each of the 8 different conditions for all 4 kinases). Only activities measured in presence of the predicted biological peptide targets of each kinase are shown (11 biological peptides for ABL1; 8 for LYN A; 20 for AKT1). The effect of increasing concentrations of inhibitors can be compared to control untreated (UNT) activity profiles (compare far left column to the two sets of three columns on the right). The sensitivity of ABL1, ABL1^{T315I}, LYN A and AKT1 kinases to imatinib and dasatinib can be assessed from changes in activity measured from their individual biological peptides. The averages of the kinase activity values measured across their biological peptides in each experimental condition are provided at the bottom-end of the panel (blue-to-black-to-yellow scale of average ATP consumptions).
(c-e) Evaluation of the effects of inhibitors on kinase activity profiles using complementary analytical methods. In (c), each 228-peptide-activity profile of each sample was first normalized against the baseline activity level measured from the 14 peptide-free well controls included in each experimental 384-well plate, and then each single peptide-derived activity level measured in presence of inhibitor was compared to the activity level found in untreated sample (i.e. all activities from all peptides in untreated sample become the baseline ‘0’ value). Results of the average of the differential ATP consumption comparing inhibitor-treated versus untreated for each biological peptide are shown as color-coded kinase activity profiles in (c). (These data were used to plot FIG. 1k.) High concentration (10 uM) of staurosporine is included as control of general shut down of kinases' activity.
In (d), entire 228-peptide activity profiles of ABL1^{T315I} comparing untreated versus imatinib- or dasatinib-treated samples were investigated for potential trends in inhibitory effects following the principles and statistical methods used in FIG. 14b. These data validate the overall lack of inhibition of ABL1^{T315I} observed with imatinib using its biological peptides in (b-c). A general dampening effect of dasatinib on ABL1^{T315I}, activity was observed (bottom graph in (d); almost all 228 peptide probes display a negative R²), which may relate to lower activity levels measured with some of the biological peptides shown in (c).
In (e), the analysis used ATP consumption levels measured in presence of peptides most robustly associated with high activity of ABL1 (derived from the analysis described in FIG. 14a, and including biological peptides). Results directly validate the gradually inhibited activity profiles found in panels (b-c).

These data also enabled cross comparing the differential sensitivity of kinases to these different drugs. For instance, the mean correlation coefficient (meanR²) relating ATP consumption and dasatinib concentration was −0.997 (SD=0.003), −0.766 (SD=0.21), −0.33 (SD=0.09) for ABL1, LYN A and ABL1^{T315I} respectively (calculated using peptides associated with kinases' highest catalytic activity, including their biological peptides). So, dasatinib had the strongest inhibitory effects on ABL1, followed by LYN A, and had some limited but measurable effect on ABL1^{T315I}. As an additional validation of these data, a formal comparison of the t-test to the Fisher-transformed correlation coefficients established from the levels of drug inhibition measured either with the high-activity peptide group, or with the 63-reference peptide group (which is one of the principal peptide control groups used to analyze HT-KAM readouts), showed that dasatinib inhibited significantly more the subsets of (biological) peptide targets of ABL1 and LYN A than the reference set (respective T-test (fisher): p=1.087e-07 and p=3.081e-06), but the effects of dasatinib did not differ significantly between these peptide groups for ABL1^{T315I} (T-test (fisher) p=0.41; which is explained by the general dampening of activity profiles in (d)). Based on experimental conditions tested here, data indicate that the IC50 concentration of dasatinib for ABL1 may be relatively higher than the anticipated 1nM (but far lower than 100 nM), while IC50(dasatinib) for LYN A is between 1 and 100 nM (and probably close to the anticipated 9 nM). Using the same series of comparisons and peptide sets, the imatinib drug could be identified as a strong inhibitor of ABL1 (mean(R²)=−0.847; SD(R²)=0.111), with no effect on ABL1³⁵ (mean(R²)=0.66; SD(R²)=0.025; which is the basis of the failure of imatinib therapy in leukemia), but with measurable, although limited, inhibitory effect on LYN A (mean(R²)=−0.462; SD(R²)=0.49). T-test to the Fisher-transformed correlation coefficients measuring levels of drug inhibition formally showed that imatinib inhibited significantly more the subsets of (biological) peptide targets of ABL1 and LYN A than the reference peptide set (p=0.00252 and p=2.054e-06, respectively). Conversely, the peptide sensor group appeared to be more activated by imatinib for ABL1^{T315I} (p=0.027), which may relate to some of the observed increased activities monitored with biological peptides in (c). (Such phenomenon was also observed for imatinib- and dasatinib-treated AKT1 (bottom heatmaps in (b-c)), while AKT-targeting drug MK2206 treatment effectively decreased AKT1 activity (data not shown) in control assays using the same analytical methods presented in (b-e).) Based on the set of drug concentrations used in this experiment, the IC50(imatinib) for ABL1 may be higher than the anticipated 0.2 uM (but far lower than 20 uM), while IC50(imatinib) for LYN A may be lower than the anticipated 100 uM (possibly close to 20 uM).

Such kind of analyses also allowed comparing the effects of different drugs on a given kinase. In particular, these results showed that dasatinib is a more potent drug than Imatinib for ABL1, and this is also true for LYN A or ABL1⁵¹. For example, the biological peptides of ABL1 revealed a stronger response to dasatinib than imatinib, which is corroborated by statistical analysis (dasatinib vs. imatinib: mean(R²)=−0.997 vs. −0.847; T-test (fisher) p=1.087e-07 vs. 0.00252). Moreover, representations in (b,c,e) indicate that the kinetics of response to drug treatment are not identical between different peptides, which suggests that the avidity for, and the phosphorylation of, particular peptide sensors may be differentially affected by an inhibitor.

These results indicate that drug-responses established from arrays of peptide probes provide the confidence and reliability that any single-peptide readout may not offer. These data also demonstrate that the HT-KAM can be used as a functional screen to assess the differential response of different kinases to inhibitors, which further illustrates the performance of the HT-KAM assay and its potential utility toward pharmacological screens. All these results directly show that biological peptides are excellent sensors to measure the activity of their cognate kinases.

FIG. 17. Measuring the effects of SFK-inhibitors using SFK's biological peptides as sensors of drug response.

(a) Activity of FYN A, HCK, LCK, and SRC kinases treated with PP2 (20 nM) and SU6656 (250 nM) inhibitors. Literature reports indicate that IC50 concentrations of PP2 and SU6656 are (5 nM; 170 nM) for FYN A; (5 nM; not available) for HCK; (4 to 30 nM; >5 uM) for LCK; (35 to 100 nM; 280 nM) for SRC. Differences in drug sensitivity can be observed between kinases. Data overall corroborate previously reported ranges of sensitivities for these drugs.
(b) Activity of HCK treated with serial dilutions of SU6656 (1nM to 100 uM). Of note, the sensitivity of HCK to SU6656 was more pronounced when measuring it with biological peptides than when monitoring it using HCK's generic control positive peptides (n=4; data not shown). In (a-b), results were processed using the same analysis described in FIG. 16c. Together, these results show that the biological peptides of SRC kinases are adequate sensors to monitor the effects of SRC kinase-inhibitors.

FIG. 18. Multi-peptide-based identification of kinases that mediate intrinsic resistance to BRAF^{V600E}-targeted therapy in colorectal cancer (CRC) cells.

(a) Schematic of the experimental procedure.
(b) Previously identified mechanisms involved in resistance to BRAF^{V600E}-targeted therapy^{3,17}. The anticipated reduction in MEK1/2 and ERK1/2 protein phosphorylation (blue shading), and increase in EGFR protein phosphorylation (red shading), served as validated knowledge to assess whether the HT-KAM platform can effectively detect MEK/ERK/EGFR kinases' activity in biological samples after vemurafenib (VEM) treatment.
(c) Baseline levels of protein and ATP concentrations comparing untreated (UNT) vs. VEM-treated WiDr cell extracts across all samples. VEM and UNT samples were comparable.
(d) Comparison of the activity levels of MAPKs, HERs, AKTs derived from kinases' biological peptide subsets (x-axis), versus those derived from peptides associated with kinases' most differential signature (y-axis; peptide sets previously computationally identified in FIGS. 10-11). The table on the right of the graph indicates the number of peptides per kinase. In the graph, the x- and y-axes represent a scale of the differential activity profile between VEM and UNT samples across all experimental repeats (total of 12 UNT and 12 VEM HT-KAM runs). Data plotted on the x-axis display the average of the activity differences across all experimental repeats and all biological peptides per kinase. Data plotted on the y-axis use kinase activity levels calculated as the difference between (i) the mean activity measured from the peptide subset that specifically differentiates the kinase in question from all other kinases, and that is associated with greater phosphorylation by the recombinant kinase in question, ‘minus’ (ii) the mean activity measured from the differential peptide subset specifically associated with lower phosphorylation by this kinase.

For example, after VEM treatment, the ‘increase’ in activity of AKT1 kinase (red mark) is +82 based on the 20 biological peptide targets of AKT1 (x-axis), and +46 based on the difference between the average activity measured from AKT1's 21 most differentially high-activity peptides ‘minus’ the average activity measured from AKT1's 6 most differentially low-activity peptides (y-axis).

The plot also incorporates activity profiles established from two different normalization methods. One normalization uses the average value of ATP consumption across 228 peptides+14 peptide-free cell extracts (circles). The other normalization uses the average across 16 Y/S/T-free peptides (squares). Both normalizations lead to overall similar outputs of VEM-UNT differential activity profiles. Correlation between peptide subsets and for each normalization is indicated below the table, and the trend line of the linear regression between all data points is shown (overall correl.=0.757).

(e) Comparison between changes in phospho-protein levels measured by western blot (top section), versus the changes in kinase activity measured with generic positive control peptides (middle section), versus the changes in kinase activity measured with their subsets of biological peptides (bottom section). Each blue-to-white-to-red color band represents the average of the difference in phospho-activities per peptide between VEM and UNT samples and across all experiments (i.e. UNT equals ‘0’; not shown). A gray side bar is drawn by the top-75% biological peptides that follow the main activity trend. Western blots serve as validation of kinases activity profiles.

(f) Formal comparison between changes in phospho-protein levels by western blot (x-axis), and the differences in levels of phosphorylation activity for individual kinases (y-axis) based on either their generic positive control peptides (triangles), or biological peptide subsets (circles), or differential peptide subsets (squares). The identity of kinases is color-coded (markers' legend are shown in between the graph and the table). We used Image J to quantify intensities of bands identified by western blots. Phospho-protein amounts (x-axis) are plotted against the mean difference in kinase activity (y-axis; VEM ‘minus’ UNT) measured with ‘n’ peptides belonging to each category (indicated in the table on the right) across all experiments. In the case of GSK3B, we used the opposite value of the western blot quantification since higher phosphorylation of GSK3B is associated with a decrease in its activity.

Results show that kinase activity values established from either biological or differential peptide subsets correlate with protein levels: circles and squares are located either in the top right quadrant (i.e. high phospho-protein amount is associated with high kinase activity after VEM; which includes: EGFR, AKT1, PDPK1, SGK1, PRKCA, PKN1/2), or in the bottom left quadrant (i.e. low phospho-protein amount is associated with low kinase activity after VEM; which includes: MEK1, ERK2, p38a, GSK3B). Unlike kinase signatures established from biological or differential peptide subsets, positive control peptides did not correlate with kinases' activity (see triangles in the top left quadrant and the bottom right quadrant; e.g. positive control peptides did not identify ERK2 and EGFR kinase as respectively less and more active after VEM treatment). This is also visible in panel (e) (phosphorylation profiles of positive control peptides are shown below western blots).

Together, results in (d-f) show that the HT-KAM system can identify and differentiate individual kinases and kinase families from each other in cell extracts, which validates the utility of the HT-KAM platform. The different biological peptide subsets of different kinases can effectively be used to measure the activity of their related kinases in biological samples.

(g) Summary of the functional state of kinases found in WiDr BRAF^{V600E} CRC cells treated with VEM using HT-KAM. Arrow linkers represent the functional connectivity between kinases, and colors indicate activity (e.g. VEM induces higher AKT activity, which leads to GSK3B phosphorylation, which reduces GSK3B activity).

FIG. 19. Levels of phosphorylation activity measured with peptides derived from functionally related proteins or pathways. We provide examples of phospho-activities measured with individual biological peptides that we purposely selected and organized by protein of origin or by signaling pathway of interest.

(a) Peptide sensors related to EGFR-signaling (EGFR, PLCG1, GAB1, CBL, GRB10).
(b) Peptide sensors related to TGFBR-signaling (SMAD2 transcription factor).
(c) Peptide sensors related to transcription factors/regulators (CREB's, FOXO's, cFOS, STAT's, SP1, JUN's, NFkB1, cMYC).
(d) Peptide sensors related to kinase proteins: MTOR, JAK's.
(e) Peptide sensors related to phosphatase proteins: CDCl25C, PTEN.

In all panels, the levels of phosphorylation activity measured with individual peptide sensors correspond to the differences in activity measured between VEM-treated and UNT-control samples. Significance per peptide (or peptide group) comparing all experimental read outs across samples and repeats is indicated (pairwise t-test p-val comparing UNT vs. VEM profiles).

Based on these observations, it is tempting to link levels of phosphorylation measured with biological peptides to the functional state of the signaling circuits these peptides originate from, and how they relate to mechanisms of VEM-resistance (e.g. EGFR, CDCl₂5C³, TGFBR¹⁸, MTOR). Some of these biological peptides also happen to be those that are ‘paradoxically’ more phosphorylated within the peptide-phospho-signatures of kinases that otherwise exhibit low activity profiles after VEM treatment (e.g. ERK2's biological peptide subset includes peptides related to EGFR-reactivation circuit (GAB1 T476, GAB1 T312, GRB10 S150 peptides) and TGFBR-pathway (SMAD2 S250 peptide); see FIG. 18e first panel on the left). It would be interesting to investigate whether such peptides are early signs of re-activation of particular kinases (e.g. re-activation of MAPK signaling contributes to insensitivity to RAF inhibition^{17,19}).

Inspection of the results for individual biological peptides demonstrate the robustness of the phospho-catalytic profiles established from cell extracts: (i) similar activity levels measured with biological peptides of similar sequences (e.g. peptides from MTOR, or JAK1, or CDKN1A, or CDCl25C), (ii) systematically higher activity levels measured with biological peptides than with their mutated/pre-phosphorylated counterparts (e.g. EGFR, PLCG1, GAB1, GRB10, STAT1, GRIN2B, ABL1, MTOR; data not shown). The phosphorylation state of many biological peptides could also be corroborated by western blots, for example for EGFR (Y1068) (see FIG. 18e third panel), MTOR (S2448) (d), and others for which western blots are not shown (e.g. upregulation of phospho-EGFR (Y869), PLCG1 (Y1253), CBL (Y774), FOXO3 (S253), and downregulation of phospho-cMYC (T58), JUN (Y170), CDCl25C (T48)).

FIG. 20. Response of BRAF^{V600E} CRC cells to targeted therapy combinations. To validate the kinase activity signatures of VEM-resistant WiDr cells found by HT-KAM screen, and as a mean to assess the role of these kinases (AKT1, PDPK1, PRKCA, SGK1) as mediators of the response to VEM and their potential value as druggable vulnerabilities in our model, we used 3-day cell viability assays to monitor the response of cells to drug combinations.

(a) Summary graph of WiDr cell growth response to VEM at 2 uM (=GI50 concentration) or 0.25 uM, and either used alone or in combination with other inhibitors. The bar graph is derived from complete cell growth response data curves (as shown in FIG. 2d or in the VEM+EVE graph shown underneath the main panel in (a)). All results were confirmed with PLX4702, a compound related to VEM (data not shown). Results show that lower than single-agent-GI50-doses resulted in effective combinatorial cell growth inhibition. These data validate that the significantly hyperactive kinases found by HT-KAM are biologically meaningful targetable vulnerabilities.
(b) Table summarizing the characteristics of WiDr cells' response to drug combinations. The significance of drug interaction (two-way ANOVA; p-val) and combination index (CI; following the Loewe Additivity model^{6,7}; arbitrary threshold: synergy CI≤0.6; additivity 0.6<CI≤1.0; average for VEM≤2 uM and 2nd drug≤IC50 concentrations) are listed.
(c) List of other drugs and additional BRAF^{V600E} cell lines intrinsically resistant to VEM that we tested to further validate kinase hits identified by HT-KAM profiling.

These results confirm that the kinases found by HT-KAM are involved in the response and resistance to VEM in BRAF^{V600E} CRC. Together with data in FIGS. 18-19 and FIG. 2a-b, these results show that a finite number of key functional dependencies can be identified as additional mechanisms participating in therapeutic resistance, which offers a selective choice of effective targets to explore. So, HT-KAM can be used as an exploratory/discovery platform to survey kinases and their activity levels, which is especially valuable in the case of diseases that cannot be defined by a single driver mutation or individual genetic dependencies such as BRAF^{V600E} CRC. As such, the HT-KAM system can help select drug candidates that target orthogonal modes of resistance with high likelihood of restoring therapeutic sensitivity, thus providing a rational approach to design combination therapies.

FIG. 21. Ranges and levels of peptide phosphorylation measured across 20 cancer cell lines.

(a) Peptide phosphorylation activity signature of cancer cell lines. Protein extracts were generated for 20 cancer cell lines. Their phosphorylation activity was tested on the HT-KAM platform. For each experimental run, the average value of ATP consumption across the 228 peptides and 14 data-points from cell extract alone (i.e. established from 14 peptide-free control wells per 384-well plate) was used for internal normalization, and then the activity per-peptide was calculated as the difference in ATP consumption between individual peptide-derived read outs and the internal mean. Next, phosphorylation activity values measured for each peptide were averaged across experimental repeats for each cell line. Finally, the phospho-catalytic activity signatures measured across the 228-peptide sensors were subjected to unsupervised hierarchical clustering. Phospho-catalytic activities are color-coded based on the relative level of activity measured in presence of each peptide for each cell line, from blue for low activity, to white for intermediate-or-mean activity, to red for high activity. This analysis showed that each cancer cell displayed a unique phospho-catalytic fingerprint. On the right side of the main heatmap, the peptide class is indicated as a red/gold/grey color streak. At the bottom of the main heatmap, the phosphorylation activity profiles of cell extracts without peptide (i.e. measured in the 14 peptide-free control wells) are shown.
(b-d) Examining the patterns of phosphorylation activity across cancer cells. The phospho-catalytic activity profile of cancer cell lines was examined for three technical variables: range of activity per peptide probe, level of phosphorylation intensity per peptide probe, and peptide class. The underlying reason is that, for an assay to best distinguish the phosphorylation activities of different samples, it would be most appropriate to rely on phospho-sensing probes that capture the widest possible dynamic range of phosphorylation activities between cells, and/or provide the overall highest level of phosphorylation activity across cells. While asking this question, we also considered the possibility that the results of these two first variables may be different depending on the class of peptide used in the HT-KAM assay, that is: biological peptides, or positive control peptide probes, or reference peptides.

In (b), results are sorted by peptide class, and then by highest to lowest range of phosphorylation activity per peptide.

In (c), the table lists the values calculated from (b) to compare the three-peptide classes.

In (d), results are sorted by highest to lowest average phosphorylation activity per peptide. Peptide classes are then grouped and counted in the lower graphs to provide a sense of which peptide class reports on the highest to lowest levels of phosphorylation activity.

Results in (b-d) show that biological peptides provide the broadest ranges and highest levels of measurable phosphorylation activities. Reference peptides provide the lowest activity measurements overall, while still providing a broad range of phospho-activities. Generic positive control peptides provide the lowest range of activities, and the measured phospho-activity levels remain lower than the overall mean activity per cell line across all cells. We conclude that, although virtually all peptides participate in defining the unique peptide-phosphorylation activity signature of every cancer cell line, the set of biological peptides are particularly well-suited catalytic activity sensors to measure and differentiate the unique functional phospho-fingerprint of cancer cells.

(e) ATP levels in peptide-free wells and protein concentrations per cell line. In the graph on the left, each bar represents the average +/−st dev of ATP concentration of cell extract alone measured across the 14 peptide-free control wells available for each experimental 384 well/plate. All samples for each cell line were tested at least three times. The range of ATP-standard across all HT-KAM assays is shown on the far right of the graph (averages and standard deviations across all HT-KAM experiments are shown). These results show that ATP profiles of all samples fit within the limits of the range of ATP standard. ATP profiles were comparable between different cell lines or samples or experimental repeats. There is no evidence for any ATPase or phosphatase contamination. So, variations in phospho-catalytic activity measured in presence of peptides were both peptide-dependent and cell-specific (results shown across all figures). As such, phospho-catalytic activity profiles can be interpreted with confidence.

FIG. 22. The Kinase Activity Signature of Cancer Cells Identifies Druggable Vulnerabilities.

(a) Kinase activity signature of cancer cell lines. The activity of kinases was calculated as the average of the phosphorylation activities measured in presence of their respective subset of biological peptides. The kinase activity profile of each cell line was deconvoluted from the corresponding peptide phosphorylation profile shown in FIG. 21a, and for individual kinases or kinase families with ≥3 biological peptides. Unsupervised hierarchical clustering was applied across cells and kinases. Each cancer cell line displayed a distinguishable kinase signature. Kinase activity is color-coded blue-to-black-to-yellow from low-to-medium-to-high activity.
(b) Pearson-correlation heatmap highlighting the functional relationship between cancer cells established from their kinase activity signature. Cells are arranged by tumor tissue of origin (MEL: melanoma; CRC: colorectal cancer; BC: breast cancer; LC: lung cancer; PC: prostate cancer).
(c-d) Comparing kinase activity and drug sensitivity of cancer cell lines. A good way to evaluate the performance of HT-KAM-defined kinase signatures is to measure the effect of a kinase inhibitor on cell growth and then correlate it to kinase activity level. This would assess whether kinase signatures are predictive of drug sensitivity and indicative of cell-specific kinase dependencies. We tested the effects of 28 kinase-inhibiting drugs (list in (c)) in 3-day dose-response cell viability assays using a core set of cell lines (A375, AU565, H3122, HCC70, HCT116, HT29, MCF7, MDA231, MDA436, SK-CO-1, SkMel2, T47D, WiDr; additional cell lines were tested on a case by case basis where it was deemed useful, such as PC9 to compare EGFR activity versus gefitinib sensitivity²⁰). We intentionally chose to test more than one inhibitor per kinase for some of the kinases in order to validate results (the 28 drugs inhibit 17 different kinases/kinase families; see table in (c)). We also further corroborated GI50 results from literature²¹⁻²³. Kinase activity levels where extracted from signatures available in (a). We then correlated cancer cells' GI50's with their kinase activity for each drug. Examples are shown in panel (d). The table in (c) provides all correlations. The negative correlation between kinase activity and drug-GI50 across cells indicates that, overall, cell lines were more susceptible to inhibitors for which they displayed higher kinase activity. These results directly verify that profiles measured by HT-KAM assays accurately predict differences in kinase activity.
(e) Correlation between kinase activity and drug sensitivity. Results from both the analysis in (c-d) and from FIG. 2g are included (total of 373 data points established from 37 inhibitors that target 22 kinases). For each kinase/drug pair, we compared the differential kinase activities and drug-GI50s across tested cancer cell lines. These results show that the kinase signature of cancer cells can reveal their actionable vulnerabilities, and thus their kinase dependencies.

Along with FIGS. 18-21, this demonstrates that the peptide phosphorylation signatures can be reasonably converted in kinase activity profiles with translational relevance. The HT-KAM platform is a practical solution to find active, druggable kinases in cell culture models.

(f-g) Controls for the comparison of A375 vs. WiDr cells. Protein concentration, baseline ATP levels, and protein expression/phosphorylation levels are provided for validation of FIG. 2g-h.

FIG. 23. Melanoma specimens.

(a) Table with details about treatments received by patients before and after the biopsy/excised biospecimen that was used for histo-pathology/diagnostic and retrospectively tested on the HT-KAM platform. Treatments (muphoran; dacarabazin; interferon (IFN; low/high dose); ipilimumab (anti-CTLA4); vemurafenib (BRAF^{V600E} inhibitor); interleukin; irradiation) are color-/letter-coded. Numbers of treatment cycles are specified. Tumor stage, BRAF status, recurrence and survival are indicated. The flash-frozen tumor biospecimen of patient #1 was a superficial spreading melanoma (SSM) excised from the trunk; patient #2: SSM metastasis from the trunk; patient #3: subcutaneous metastasis from a recurrent cutaneous tumor from the leg; patient #4: nodular melanoma metastasis from the leg; patient #5: cutaneous metastasis from the cubital fossa; patient #6: cutaneous metastasis from the neck; patient #7: inguinal lymph node metastasis (axillary lymphadenectomy of positive sentinel); patient #8: cutaneous metastasis from a recurrent cutaneous tumor of the temporal area, patient #9: cutaneous metastasis from a recurrent cutaneous tumor located on the back.
(b) Baseline ATP levels measured across HT-KAM assays, and protein concentrations from tissue extracts. Levels are comparable between tumors and experiments.

FIG. 24. Analysis of the phospho-catalytic signatures of tumors.

(a) Peptide phosphorylation activity signature of melanoma specimens. Unsupervised clustering of 36 activity profiles measured across 228 peptides is shown (9 patient tumor tissues tested in 4 independent technical replicates).
(b-h) Principal component analysis (PCA). We investigated the potential association between ‘variables of interest’ and principal components (PCs) that define individual phospho-signatures (linear regression, overall fit of univariate model PC(i) for variable (j)). The table in (b) provides a summary of the results visually represented in graphs (c-g).
In (c), results show that replicate runs from the same patient sample were significantly similar (9 patients are shown as 9 different colors, with 4 dots per patient for the 4 experimental replicates), with an association between patient ID and PC1 of p=1.44E-06. In (d), results show that days at which assays were run were not associated with the primary PCs of melanoma kinomes (3 different days of experimentation are shown as 3 different colors; p>0.05). So, the results of the PCA from the two technical variables in (c-d) demonstrate the excellent performance and high reproducibility of the HT-KAM system (i.e. experimental procedure, instrumentation, data analysis).
In (e-f), results show that survival status (f) and recurrence status (g) were highly associated with the PCs of the kinome signatures (respectively PC1:p=5.14E-06 and PC2:p=0.028; and PC1:p=2.31E-07). So, the results of the PCA from the two clinical variables in (f-g) reveal the strong predictive and prognostic value of the phospho-catalytic signature of tumors.
In (g), results show that PCs of melanoma phospho-signatures do not associate with BRAF mutational status. This demonstrates that, even if a BRAF^{V600E} mutation is a dominant oncogenic driver, HT-KAM effectively discerns different types of BRAF^{V600E} tumors depending on other critical variables that the phospho-signatures of tumors reflect. HT-KAM differentiates fatal BRAF^{V600E}-positive conditions (patients #4,5,8,9) from non-fatal BRAF^{V600E}-positive conditions (patients #3,7). And similarly, HT-KAM distinguishes fatal non-BRAF^{V600E} conditions (patient #2) from non-fatal non-BRAF^{V600E} conditions (patients #1,6).
In (h), we overlaid PC results from (e-g) and annotated patients groups for outcome and therapeutic resistance. This shows that the PCs from VEM treated-but-resistant patients (#4,5,9) cluster together, indicating that tumor phospho-signatures reflect patients' unresponsiveness to BRAF-targeted therapy. The strong association between BRAF-therapy resistant lethal melanoma tumors and the PCs of their phospho-signatures fully validates the results generated from the clustering analysis available in FIG. 3b.

The results of the PCA shown in (b-g) were established from phospho-signatures normalized to the 228-peptides+14 peptide-free readouts, and were closely recapitulated when signatures were normalized using the 63-reference peptides, or the 14 peptide-free tissue extract alone readouts, or 16-Y/S/T-free peptides, or when using raw ATP-consumption data (data not shown). Collectively, this formal investigation of the PCs of tumor phospho-signatures demonstrates the robustness of the HT-KAM system, and the reliability of its output to map the phospho-catalytic signatures of tumor tissues.

FIG. 25. Identifying the predictive peptide signature of patients with poor survival outcome, including BRAF^{V600E}-mutated patients resistant to BRAF-therapy. Since survival is a significant clinical variable identified from patients' phospho-signatures (clustering analysis in FIG. 3b, and PCA in FIG. 24), we asked whether we could find peptides that would qualify as best predictors of patients' survival outcome. To do so, we applied two methods.

(a-c) Method #1: analysis of peptide phosphorylation signatures using dual significance threshold selection. In (a), the waterfall plot shows the differential activity profile across all 228-peptides comparing patients #1,3,6,7 (alive) versus patients #2,4,5,8,9 (dead). Significance values (t-test and Wilcoxon rank sum test; FDR-adjusted or not) are provided underneath the graph. Peptide phosphorylation signatures were normalized to the 228-peptides+14 peptide-free readouts. In (b), peptide phosphorylation activities were selected when concurrently passing FDR-adjusted t-test p<0.05 and Wilcoxon rank sum test p<0.05. This rigorous dual selection threshold identified 34 peptides as the most significantly differentially phosphorylated peptides associated with poor survival. In (c), peptide phosphorylation activities shown in (b) are detailed across all patient signatures. Two thirds of the peptides are biological peptides (listed and color-coded on the right side of the heatmap), most of which are associated with higher phosphorylation activity in tumors from patients with poor survival outcome. We further validated these data using alternative normalization schemes (data not shown), and by applying this same computational analysis to identify peptides qualifying as best predictors of recurrence (identifying an overlapping set of 27 peptides; data not shown).
(d-e) Method #2: analysis of peptide phosphorylation signatures using top-25%-activity threshold selection. The top 25% peptides displaying phospho-activities that were most variable between all patients and all experimental repeats were selected. Panel (d) shows the unsupervised hierarchical clustering of these 57 most differential signals (same normalization as in a-c). In (e), the waterfall plot shows the differential activity per peptide across these 57 peptides and between patient groups (differential survival outcome as black bars; differential recurrence outcome in violet; differential BRAF status in blue; data sorted by highest to lowest differential activities comparing survival outcome).

Results in (a-e) show that the most significant and consistently high signals associated with fatal outcome were measured with biological peptides such as SMAD2 S465 and S245 and S250, KHDRBS1/SAM68 Y440 and Y435, MTOR T2446 and S2448, CDKN1A/p21 T145 and S146, BRCA1 S988 and T509, ABL1 Y226 (corresponding to Y245 in another ABL1 isoform), FCGR2B Y292, or CHEK1 S280. Likewise, peptide sensors associated with consistently low activity in fatal outcome were biological peptides NOTCH2 S2070, JUN Y170, TERT Y707, GAB1 Y627, and reference peptides from modified biological peptides PA_128, PA_134, or PA_230. Interestingly, the phospho-signatures of poor survival outcome included peptide sensors related to kinases such as SFK's, MTOR, or TGFBR-signaling, some of which were found to confer acquired resistance to BRAF or MEK inhibitors in cell culture models of melanoma²⁴⁻²⁸. Phospho-catalytic activities measured with peptides of related sequences behaved similarly and were useful internal controls validating assay repeatability (e.g. MTOR T2446 and S2448; CDKN1A T145 and S146; CDK5 Y15 and generic positive control PA_240 which is originally derived from CDK1 Y15 and is mostly conserved between these two CDK's). So, the peptides included in the HT-KAM platform are robust sensors that can be used to generate clinically valuable signatures to diagnose cancer specimens.

FIG. 26. Identifying the kinase signature of patient tumors. Determining the activity of kinases in tumors could reveal tractable candidates for therapeutic interventions. So, we explored the potential clinical utility of the kinase activity signatures of melanomas.

(a) Waterfall plot showing the differential activity of kinases between patient groups. For each tumor, the activity of kinases with ≥4 biological peptides was calculated as the average of the phosphorylation activities measured in presence of their respective biological peptide subsets across all experimental repeats. Next, we calculated the mean activity per kinase for each patient group, and then plotted the differential activity between groups of interests, i.e. survival/black bars, recurrence/violet, BRAF status/blue. Significance was calculated using all experimental repeats between patient groups (shown underneath the graph).
(b) Differential kinase activity signature of tumors. Kinase activity profiles were mean-centered per tumor tissue and then mean-centered across patient tumors. Semi-supervised hierarchical clustering was applied across the 60 individual kinases or kinase families. As a validation of the results in (a-b), the differential activities of some of the kinases we found in patient tumors are corroborated by gene over-expression screen for RAF-inhibitor resistance in melanoma cell lines 25 (e.g. upregulated kinase activity of PRKC(E), RAF(1), MAP3K8/COT1, PAK(1)).
(c) Comparison of the activity of ABL1, AKT1, ERK2, HCK and p38a deconvoluted from kinases' biological peptide subsets (x-axis), versus activity levels converted from kinases' differential peptide signature (y-axis; method explained in FIGS. 11, 18d). Results show good concordance across kinases and tumor groups (e.g. the correlation between kinases' activity related to survival (circles) was 0.847).
(d) Validation of differential kinase signatures using enrichment analysis. We applied an enrichment analysis (EASE—Fisher one-sided test; p<0.05) to identify kinases whose biological peptides were most represented among the 34 peptides most significantly associated with survival outcome (identified in FIG. 25b-c). Results are shown in the table, and formally identify AKT's, PIM's, and RPS6KB's as most significantly related to the melanoma survival outcome phospho-signatures.
(e) Differential biological peptide-activity profiles comparing different patient groups. We chose to show AKT, PIM, RPS6KB, and GSK3B kinase families because analyses in (a-b,d) showed their changes were most significant and most profound (increase or decrease). Significance per peptide across patients groups (grey scale) are shown. The bottom graph compares the kinase profiles of BRAF^{V600E} tumors from patients retrospectively known to survive (#3,7) versus patients who died (#4,5,8,9; which includes all patients who did not respond to BRAF-therapy). This means that the intrinsic vulnerabilities of these tumors were not inhibited, and that alternative treatment options would have been available at the time of biopsy based on kinase signatures revealed by HT-KAM.

Together, these results demonstrate that differences in the activity of kinases can be measured within a tumor biospecimen (i.e. for each patient), and between different tumors (i.e. across patients and their individual malignancies). As well, significant changes in levels of kinase activity can be identified between different groups of patients (e.g. survival outcome). So, the HT-KAM assay successfully maps the oncogenic kinase signatures of tumor tissues, and reveals the significantly hyperactive kinases that are most predictive of poor outcome. Accordingly, HT-KAM identifies druggable kinase vulnerabilities most tractable to treat patients with highest likelihood of recurrence and poorest survival outcome, including patients who are unresponsive to BRAF-therapy. Specifically, our results indicate that AKT1, PIM1 and RPS6KB1 kinases are most conserved and overly active in poor outcome melanoma.

FIG. 27. Analysis of gene expression data as an indirect mean to corroborate phospho-catalytic signatures. Even though gene expression is a relatively distant factor influencing the oncogenic activity of kinase enzymes and phospho-signaling circuits^{29,30}, we asked whether melanoma patients with poorer outcome available in the TCGA resource¹⁴ displayed changes in mRNA levels that match kinases we found by HT-KAM. We confirmed that patients who express high levels of AKT1 or PIM1 displayed significantly worse overall survival or disease-free survival, as shown in (a-b) and (c-e). Kaplan-Meier curves and logrank t-test p-values are provided in (a,c). Not shown here, we also found from the TCGA data set: (i) co-occurrence of AKT1 and RPS6KB1 mRNA alteration in poor outcome melanoma (log odds ratio of +2.66; p<0.001); (ii) up-regulation of CDKN1A mRNA expression in worse progression-free survival which is interesting since CDKN1A-derived peptide sensors are among top predictors of poor outcome (see FIG. 25c).

FIG. 28. Response of BRAF^{V600E} melanoma cell lines to drug combinations.

(a) Summary of possible cell responses to the combination of two drugs. The heatmap on the left represents the experimental effects of combining increasing concentrations of drug A+B as changes in cancer cell growth. The middle graph is a conceptual representation of how differences in effects of drugs alone or combined can be interpreted as either synergistic, additive, or antagonistic (derived from¹⁰). The heatmap on the right represents the calculated combination index from all experimental cell growth data points. We decided to analyze experimental profiles using the Bliss Independence model to calculate combination indices (CI) and avoid inaccuracies from dose-effect curve estimations (CI=log 2 (Eab/(Ea*Eb); synergy CI<0; additivity CI=0; antagonism CI>0) 10-12.
(b) Maps of cell growth responses (left) and combination indices (right). We tested the dependency of BRAF^{V600E} melanoma cell lines for kinases found as differentially active by HT-KAM in patient tumors (FIG. 26a,b). We included everolimus or trametinib (bottom two rows) as experimental ‘positive’ controls and for side-by-side comparison of the effects of other kinase-targeting drugs we tested. Arrows located above and on the left of each cell growth heatmaps serve as indicators of GI50 concentrations for each drug alone. The GI50s of A375 maintained in 5% or 0.25% FBS media for drug alone were: VEM (0.15 uM; 0.02 uM); MK2206 (10 uM; 2.5 uM); AZD1208 (12.5 uM; 12.5 uM); LY2584702 (50 uM; >200 uM); PF-4708671 (32 uM; 16 uM); 1-Azakenpaullone (20 uM; 2.5 uM); BI-D1870 (12.5 uM; >50 uM); everolimus (5 uM; 10 uM); trametinib (5 nM; 2.5 nM). The GI50s of Sk-Mel-28 maintained in 5% or 0.25% FBS media for drug alone were: VEM (0.63 uM; 0.31 uM); MK2206 (2.5 uM; 5 uM); AZD1208 (25 uM; 25 uM); LY2584702 (100 uM; 100 uM); PF-4708671 (32 uM; 16 uM); 1-Azakenpaullone (20 uM; 10 uM); BI-D1870 (25 uM; 12.5 uM); everolimus (10 uM; 10 uM); trametinib (10nM; 10nM).

Results of growth responses (left) and combination indices (right) show that strong growth inhibitory effects were found when combining VEM with inhibitors of RPS6KB (LY2584702 or PF-4708671) or PIM kinases (AZD1208). Overall, synergistic effects of these drugs were superior to, or at least as good as, those of drugs targeting MTOR (everolimus) or MEK (trametinib) that are currently used in the clinic, but whose effects in patients remain variable and often transient.

Our results also show that other kinases whose catalytic activities were originally identified by HT-KAM as less highly or less significantly upregulated than RPS6KB, PIM or AKT kinases in BRAF^{V600E} melanoma tumors were indeed less effective therapeutic targets. This is exemplified by RPS6KA inhibition (additive effects only of the BI-D1870 drug; see heatmaps), or PKC inhibition (limited additivity effects of Go6983 or Sotrastaurin; data not shown).

Furthermore, we found that inhibiting the GSK3B kinase, which is a tumor suppressor kinase we identified as systematically less catalytically active in melanoma tumors of poor outcome patients, resulted in antagonizing the effects of VEM treatment, which means that melanoma cells became more resistant to BRAF^{V600E}-targeting drug when it was combined with the GSK3B inhibitor 1-Azakenpaullone, as visually noticeable from the patterns of cell growth and CI in heatmaps. This also strongly suggests that some of the ‘good’ effects of VEM rely in part on ‘proper’ kinase activity of the GSK3B tumor suppressor.

(c-e) Evaluating the effects of drug combinations on melanoma cell death. We used Fluorescence-Activated Cell Sorting (FACS) and measured cell death in melanoma cells treated at GI50 concentration of drug alone in 5% and 0.25% FBS culture conditions. Panel (c) shows examples of flow cytometry profiles obtained for Sk-Mel-28. Graphs in (d-e) show percentages of apoptosis for Sk-Mel-28 and A375 are graphically (averages of cell death measured in 5% or 0.25% FBS conditions). Numbers above grey bars in (d-e) indicate gain or loss in apoptosis when combining VEM with a 2nd kinase-targeting drug.

Increase in apoptosis was systematically found when combining VEM with inhibitors of RPS6KB or PIM, and these effects were overall more potent than when combined with MTOR or MEK targeting drugs. Decrease in apoptosis was found when combining VEM with GSK3B inhibitor (i.e. GSK3B inhibition rescues BRAF^{V600E} melanoma cells from cell death upon VEM treatment). Highest level of apoptotic cell death was induced by RPS6KB-targeting combined with VEM in Sk-Mel-28, thus achieving a critical endpoint in the perspective of eradicating tumor cells.

Altogether, these results demonstrate that the top kinase hits identified in patient tumors as predictive of their therapeutic failure (see FIG. 3c-e and FIG. 26) correspond to central kinase dependencies in BRAF^{V600E} melanoma cells. We conclude that inhibitors of RPS6KB, PIM or AKT are strong therapeutic candidates to restore therapeutic sensitivity. The HT-KAM platform successfully identifies new exploitable vulnerabilities of melanoma tumors based on the kinome profiling of patient tumor tissues.

FIG. 29. A375 expressing a constitutively active AKT1 oncogenic kinase remain sensitive to RPS6KB- or PIM-targeting. We exogenously expressed myrAKT1 (i.e. a constitutively active oncogenic AKT1) in A375 cells. We then tested the sensitivity of A375 myrAKT1 cells to drug combinations.

(a) Control for myrAKT1 expression. myrAKT1-expressing A375 cells were significantly less sensitive to VEM than their control counterpart (p=2.77E-06; 65% increase in GI50).
(b-c) Cell response of A375 myrAKT1 cells to drug treatments (same experimental settings and analysis as in FIG. 28). Cell growth is shown in (b), cell death is shown in (c).
(d) Ranking treatment effects by comparing CI values between melanoma cell lines. Data from both FIGS. 28-29 are included.

Results indicated that BRAF^{V600E} melanoma cells that acquired the expression of the AKT oncogene remained sensitive to combinations of VEM with inhibitors of RPS6KB or PIM kinases. As well, mimicking the loss of tumor suppressive activity by inhibiting GSK3B in myrAKT1-expressing cells to recapitulate the coordinated inactivation of GSK3B kinase and hyper-activation of AKT1 found in tissues from BRAF^{V600E} melanoma patients with poorest outcome, led to strong resistance to VEM treatment. The growth response of myrAKT1-expressing A375 cells treated with combinations of VEM+MTOR or MEK targeting drugs was comparable to wild type A375 cells. These results support the notion that these multiple oncogenic phospho-signaling hubs can function independently of each other's. These kinases are suitable alternative targets to restore response in melanoma cells.

FIG. 30. Restoring therapeutic sensitivity by targeting the RPS6KB kinase in melanoma tumor cells from patients that acquired resistance to BRAF^{V600E}-therapy.

(a-b) Phospho-RPS6KB profiles of two patient-derived xenografts (PDXs) tumor tissues (a) and related cell lines (b). PDXs were previously^{4,5} established from patients refractory to BRAF^{V600E}-therapy. A limited number of primary cell lines were derived from these PDX's.
(c) Sensitivity of primary melanoma cell lines to RPS6KB-inhibitor PF-4708671 in 3-week colony formation assays.

The PDX tumor M032R6.X1 displayed high levels of RPS6KB1 in tumor tissue, and the PDX-derived cell line M032R6.X1. CL maintained high levels of RPS6KB1, and the cell line M032R6.X1. CL was sensitive to RPS6KB1 targeting. This corresponds to data shown in FIG. 3i.

Conversely, the PDX tumor M061R.X1 did not display high levels of RPS6KB1 in tumor tissue, and the PDX-derived cell line M061R.X1. CL also did not display high levels of RPS6KB1, and the cell line M061R.X1. CL was not sensitive to RPS6KB1 targeting. This can be considered as a control for results in FIG. 3i.

The results indicate that targeting RPS6KB can be a successful therapeutic intervention in BRAF^{V600E} melanoma tumors/tumor cells where RPS6KB1 is elevated. Such vulnerability may be valuable to restore therapeutic sensitivity in patients who do not respond to, or relapse from, current therapies.

### DETAILED DESCRIPTION OF THE INVENTION

The disclosure is based, in part, on the discovery of an effective, sensitive assay for determining a phospho-kinase activity profile of cells, e.g., tumor cells, or other biological samples in which it is desirable to evaluate kinase activity. The method comprises using a panel of sensor peptides that comprise biologiocal substrate regions for different kinases. Evaluation of all of the members of the panel provide an indication of the kinase pathways that are active in the sample. In one embodiment, provided herein is a 228-peptide panel developled to detect the activity of over 60 kinases/kinase families, including ABL, AKT, CDK, EGFR, GSK3B, MAPK, or SRC. Such a panel provides a method of defining new mechanisms of resistance to targeted therapy, e.g., BRAF^{V600E}-targeted therapy in colorectal cancer, can identify new druggable targets, e.g., RPS6KB1 and PIM1 as new druggable vulnerabilities predictive of poor outcome in BRAF^{V600E} melanomas patient; and otherwise provides for comprehensive evaluation of kinase activity in cancer and in other complex or previously uncharacterized samples of interest to identify kinase type.

Accordingly, provided herein are panels of sensor peptides that can be used in combination to assay for the activities of multiple kinases at the same time to identify the kinase activity and/or determine the kinase signature of a biological sample of interest, e.g., a cancer sample obtained from a patient. The invention further comprises methods of assigning a kinase activity identified in a biological sample, e.g., a cancer sample, to a kinase family and/or of identifying the kinase activity as belonging to a particular kinase. Such information can be used, for example, to further characterize the kinases/kinase activities from biological samples of interest. In some embodiments, e.g., evaluation of kinase activity in cancer or other disease states, analysis of a combinatorial peptide panel in accordance with the invention can be used to develop therapeutic strategies or for further development of drug candidates.

A “sensor” peptide in the context of this invention refers to any peptide that is contained in a kinase peptide activity panel as described herein which is used to assess kinase activity. These include biological sensor peptides and sensor peptides that have mutations relative to the naturally occurring sequences that influence kinase activity. As used herein, the terms “kinase activity” and “phosphorylation activity” are used interchangeably to refer to the ability of a phosphokinase to phosphorylate a sensor peptide.

The term “peptide” is used herein to refer to a polymer of amino acid residues. The term applies to amino acid polymers in which one or more amino acid residue is an artificial chemical mimetic of a corresponding naturally occurring amino acid, as well as to naturally occurring amino acid polymers and non-naturally occurring amino acid polymers. Thus, a peptide for used in a panel of peptides to assess kinase activity as described herein can comprise naturally occurring and/or synthetic amino acids, including analogs and amino acid mimetics that function in a manner similar to the naturally occurring amino acids. “Naturally occurring” amino acids are those encoded by the genetic code, as well as those amino acids that are later modified, e.g., hydroxyproline, γ-carboxyglutamate, and O-phosphoserine. “Synthetic amino acids” or “amino acid analogs” refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified polypeptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. Amino acid is also meant to include —amino acids having L or D configuration at the α-carbon.

Amino acids may be referred to herein by either their commonly known three letter symbols or by the one-letter symbols recommended by the IUPAC-IUB Biochemical Nomenclature Commission.

### Biological Sensor Peptides

In the context of this disclosure, a “biological sensor peptide” refers to a peptide that comprises a substrate region that is derived from a naturally occurring substrate region. Thus, a biological sensor peptide as used herein refers to a peptide a known substrate region phosphorylated by a kinase belonging to a kinase family. Such substrate regions can be identified, e.g., from the PhosphoAtlas or PhosphoSitePlus databases (see, e.g., Olow et al., “An Atlas of the Human Kinome Reveals the Mutational Landscape Underlying Dysregulated Phosphorylation Cascade in Cancer” Cancer Res. 76(7):1733-45, Apr. 1, 2016; Epub 2016 Feb. 26; and Hornbeck et al., “PhosphositePLus, 2014: Mutations, PTMs and recalibrations. Nucl. Acids Res. 43:D512-D520, 2015).

Biological sensor peptide can vary in length, e.g., from 7 to 25 amino acids in length, or from 9 to 21 amino acids in length. In some embodiments, a peptide may be up to 50 amino acids in length, or even up to 100 amino acids in length and at least 6 amino acids in length. In some embodiments, a biological peptide is 9, 10, 11, or 12 amino acids in length. In some embodiments, a biological sensor peptide is 11 amino acids in length. In some embodiments, biological peptides can comprise additional amino acids at the C-terminal and/or N-terminal ends of the peptide. For example, an 11-mer may comprise 9 amino acids from the naturally occurring substrate region sequence and 2 amino aicds, e.g., GC at the C-terminus or CG at the N-terminus. In other embodiments, the biological peptide does not contain a GC or CG tag at the end terminus.

Although the sequence of a biological sensor peptide is typically identical to the corresponding region of the native substrate phosphorylation site, in some embodiments, the biological sensor peptide may contain one or more amino acid residues at the end of the peptide, relative to the native sequence, that does not influence kinase activity, as explained in the preceding paragraph. In further embodiments, a biological sensor peptide may contain an amino acid change relative to the sequence of a naturally occurring substrate region that does not influence activity of the kinase that phosphorylate that region. In some embodiments, a biological peptide may have a conservative substitution that does not influence kinase activity. In some embodiments, a biological sensor peptide may include modified amino acids. For example, modifying a peptide to have a pre-phosphorylated peptide on one Y residue may make it easier for a kinase to phosphorylate a ‘free’ Y nearby within that peptide sequence. This is also applicable to other post-translational modifications that may be biologically relevant and thus, a peptide that mimics such a state may improve the sensitivity/specificity/differentiability of detection for one or more kinases.

The number of biological sensor peptides employed in the panel per kinase is variable, but the panel comprises a plurality of biological sensor peptides for the majority of the kinases to be represented in the panel. For example, in some embodiments, a panel of comprises, 8, 9, 10, 11, 12, 13, 14, or 15 biological sensor peptide per kinase, but may range from 3 to 50 biological sensor peptides per kinase. In some embodiments, a panel of kinases may employ at least 4 biological sensor peptides per kinase. In some embodiments, the number of biological sensor peptides per kinase that is included in a panel is 5, 6, 7, 8, 9, 10, 11, 12, 13, 14, 16, 16, 17, 18, 19, or 20. In some embodiments, the number of biological sensor peptides per family ranges from 4 to 20 or more.

In some embodiments, a panel of sensor peptide comprises at least 15 biological sensor peptides per the majority of kinase families to be represented in the panel. In some embodiments, at least 10 biological sensor peptide, or at least 15 biological sensor peptides are included per kinase family represented in the panel. In some embodiments, nor more than 50 peptides is included per kinase family.

### Control Sensor Peptides

Various control sensor peptides are included in a peptide panel of the present invention for experimental controls and statistical analysis, as detailed in the Examples section and accompanying figures and figure descriptions. The controls, also referred to herein as reference peptides, include generic positive controls, and mutated peptides, in which a naturally occurring substrate region is mutated such that a known kinase that phosphorylates the substrate in nature is exhibits modified activity towards the mutant peptide, e.g., is inactive against the mutated peptide. Mutated control sensor peptides provide increased confidence in the assay and improves analysis/interpretation and visualization of data from the screening assay. For example, a mutated reference peptide may contain an amino acid substitution at a phosphorylatable tyrosine, serine, or threonine, e.g., a tyrosine, serine, or threonine, may be substituted with a glycine or other residue that is not longer able to be phosphorylated. In some embodiments, a control peptide may have a phosphorylatable site that is pre-phosphorylated to render it insensitive to kinases that may be present in a given sample. Control peptides may also include peptides that have random peptide sequences. Finally, know generic peptides that can serve as positive controls are available, including commercially available. Such peptides may also be included in the panel as further controls.

### Sensor Peptide Panels

A ‘sensor peptide panel” or “kinase activity panel” as used herein refers to an assemblage of peptides that collectively provides phosphorylation activity data (for a sample of interest being evaluated to characterize the kinases in the sample) for multiple kinases at the same time and thus is sufficient to assign kinase activity in the sample to a kinase family, or one or more specific kinases; or can be used to characterize kinases in a biological sample of interest. In a kinase activity assay panel of the present invention, it is the combination of different kinase-specific sets of peptides that provides the read out of activity for multiple kinases at once.

Further, even though many kinases may have few identified target sites or even substrate protein targets, a kinase activity panel assessment as described herein can be used to identify peptides that best match a kinase and thus provide greater insight into the kinase function.

In some embodiments, a kinase activity panel of the present invention comprises a multiplicity of sensor peptides to determine kinase activity for at least two, at least three, at least four, or at least five, or more kinases families. In some embodiments, a kinase activity panel of the present invention comprises a multiplicity of sensor peptides to determine kinase activity for at least ten, at least fifteen, at least twenty, at least twenty five, or more kinase families. In some embodiments, a kinase activity panel of the present invention comprises a multiplicity of sensor peptides to determine kinase activity for at least two, at least three, at least four, or at least five, or more specific kinases. In some embodiments, a kinase activity panel of the present invention comprises a multiplicity of sensor peptides to determine kinase activity for at least ten, at least fifteen, at least twenty, at least twenty five, or more specific kinases. Accordingly, a panel may comprise any number of peptides. Indeed, a panel and assay of the present invention is modular by design in that users can adapt panels and assay conditions to their needs. Indeed, in some embodiments, a set of peptide sensors may be employed to study particular kinases under specific conditions, e.g., pH, ion, etc.; however, the panel still employs multiple peptides to capture kinase activity for multiple kinases.

In some embodiments, a kinase activity panel of the present invention can comprise a multiplicity of sensor peptides to determine kinase activity for multiple kinases, e.g., at least five, at least ten, at least fifteen, at least twenty, at least thirty, at least forty, at least fifty, or more, kinases selected from the group consisting of ABL1, ABL1(H396P), ABL1(Q252H), ABL1(T315I), BCR-ABL (fusion gene, Philadelphia translocation), ABL2, BLK, BRK (PTK6), FGR, FRK, FYN, HCK, LCK, LYNA, LYNB, SRC, SRMS, YES1, CSK, BTK, BTK(E41K), FER (TYK3), FES, ITK, LTK (TYK1), SYK, TEC, TEK (TIE2), TXK, TYRO3, ZAP70, JAK1, JAK2, JAK3, TYK2, PTK2 (FAK, FAK1), PTK2B (FAK2, PYK2), ALK, AXL, AXL(R499C), CSF1R, DDR1 (NTRK4, RTK6, PTK3), DDR2 (NTRKR3, TYRO10), EGFR, ERBB2, ERBB3 (in its dimeric state with another HER fam. member), ERBB4, FGFR1, FGFR2, FGFR2(N549H), FGFR3, FGFR4, FLT1 (VEGFR1), FLT3, FLT3(D835Y), FLT4 (VEGFR3), KDR (VEGFR2), IGF1R, INSRR (IRR), INSR, ROS1, KIT (SCFR), MET, PDGFRA, PDGFRB, RET, ROR1 (ROR1 (NTRKR1), ROR2 (NTRKR2)), ROR2 (NTRKR2), EPHA1, EPHA2, EPHA3, EPHA4, EPHA5, EPHA8, EPHB1, EPHB2, EPHB3, EPHB4, RON (MSTIR), NTRK1 (TRKA), NTRK2 (TRKB), NTRK3 (TRKC), MERTK (MER), WEE1 (WEE1A), WEE2 (WEE1B), MUSK, ACVR1 (ACVRIA, ALK2), ACVR1B (ALK4), TGFBR1 (ALK5), TGFBR2, BMPR1B (ALK6), AKT1, AKT1(E17K), AKT2, AKT2(E17K), AKT3, AKT3(E17K), AKT3(G171R), ARAF, BRAF, RAF1 (cRAF), ATM, ATR, AURKA, AURKB, AURKC, CHEK1, CHEK2, PRKDC (DNA-PKcs, DNAPK), PLK1, PLK3, BRSK1, BRSK2, BUB1, CI1 orf7, CAMK1, CAMK1beta, CAMK1delta, CAMK1gamma, CAMK2, CAMK2A, CAMK2B, CAMK2D, CAMK2G, CAMK4, CAMKK1, CDCl42BPA (MRCK alpha), CDCl42BPB (MRCK beta), CDK1 (CDCl₂), CDK2, CDK3, CDK4, CDK5, CDK6, CDK7, CDK8, CDK9, CDK11, CHUK (IKK alpha), IKBKB (IKK beta), IKBKE (IKK-E), CLK1, CLK2, CSNK1A1 (CK1A), CSNK1D (CK1D), CSNK2A1 (CK2A1), CSNK2A2 (CK2A2), CSNK2B (CK2B), DAPK3 (ZIPK), DMPK (DM1), DYRK1A, DYRK2, IRAK1, IRAK4, EIF2AK1, EIF2AK2, EIF2AK3 (PEK), EIF2AK4, GRK3 (ADRBK2), GRK5, GSK3A, GSK3B, HIPK2, ILK (p59integrin-linked kinase RAF-like kinase), STK3 (MST2, KRS1), STK4 (MST1, KRS2), STK10 (LOK), STK11 (LKB), MTOR (FRAP1), KSR1, KSR1(A635F), KSR1(L639F), KSR2, KSR2(R676S), MAPK1 (ERK2), MAPK3 (ERK1), MAPK7 (ERK5/6), MAPK8 (JNK1), MAPK9 (JNK2), MAPK10 (JNK3), MAPK11 (p38b), MAPK12 (p38g), MAPK13 (p38d), MAPK14 (p38a), MAP2K1 (MEK1, MKK1, MAPKK1), MAP2K2 (MEK2, MKK2, MAPKK2), MAP2K4 (MEK4, MKK4, MAPKK4, JNKK1), MAP2K7 (MEK7, MKK7, MAPKK7, JNKK2), MAP3K1 (MEKK1, MAPKKK1), MAP3K7 (MEKK7, TAK1), MAP3K8 (COT), MAP3K14 (NIK), MAP3K17 (TAOK2, PSK1), MAP4K2, MAP4K4 (MEKKK4), MAP4K5, MAP4K6 (MINK), MAPKAPK2 (MK2), MAPKAPK3 (MK3), MAPKAPK5 (MK5), MARK1, MARK2 (EMK1), MARK3 (CTAK1), PKMYT1 (MYTi), NDR1 (STK38), NDR2 (STK38L), NEK1, NEK2, NEK6, NEK7, NLK, NME1 (NM23, NDPK-A), NME2 (NM23B, NDPK-B), NME1-NME2, NUAK1, PAK1, PAK2 (p21 (RAC1) activated kinase 2), PAK3, PAK4, PAK7, PDK1 (pyruvate dehydrogenase kinase 1, GeneID: 5163), PDPK1 (3-phosphoinositide dependent protein kinase 1, GeneID: 5170), PHKG1, PIM1, PIM2, PIM3, PKN1 (PRK1), PKN2 (PRK2), PRKAA1 (AMPKa1), PRKAA2 (AMPKa2), PRKACA (PKA C-alpha, PKACA=protein kinase cAMP-activated (PKA) catalytic subunit alpha), PRKACB (PKA C-beta, PKACB=protein kinase cAMP-activated (PKA) catalytic subunit beta), PRKACG (PKA C-gamma, PKACG=protein kinase cAMP-activated (PKA) catalytic subunit gamma), PRKCA (PKC alpha, PKCA), PRKCB (PKC beta, PKCB), PRKCD (PKC delta, PKCD), PRKCE (PKC epsilon, PKCE), PRKCG (PKC gamma, PKCG), PRKCH (PKC eta, PKCL), PRKCI (PKC iota, PKCI), PRKCM (PKC mu, PKCM), PRKCN (PKC nu), PRKCQ (PKC theta), PRKCZ (PKC zeta), PRKD1 (PKD1, PKD), PRKD2 (PKD2), PRKD3 (PKD3), PRKG1 (PKG 1, subunits I-alpha & I-beta, PRKGR1, PRKG1=protein kinase, cGMP-dependent (PKG), type I), PRKG2 (PKG 2, PRKGR2, PRKG2=protein kinase, cGMP-dependent (PKG), type II), PRKRIR (THAP12, DAP4, P52rIPK), ROCK1, ROCK2, RPS6KA1 (RSK1, p90RSK), RPS6KA2 (RSK3, p90RSK2, S6K-alpha-2), RPS6KA3 (RSK2, p90RSK2, S6K-alpha-3), RPS6KA4 (MSK2, S6K-alpha-4), RPS6KA5 (MSK1), RPS6KA6 (RSK4, p90RSK6, S6K-alpha-6), RPS6KB1 (S6K1, p70S6K, S6K-beta-1), RPS6KB2 (S6K2, p70S6Kb, S6K-beta-2), SGK1 (SGK), SGK2, SGK3 (SGKL), SMG1, TAF1, TBK1 (NAK, T2K), TP53RK (PRPK), TSSK1, TSSK2, TSSK4, VRK1, ABL fam. (=ABL1, ABL1(H396P), ABL1(Q252H), ABL1(T315I), BCR-ABL, ABL2), SRC fam. (BRK (PTK6), FGR, FRK, FYN, HCK, LCK, LYNA, LYNB, SRC, SRMS, YES1), JAK fam. (JAK1, JAK2, JAK3, TYK2), PTK2/FAK fam. (PTK2/FAK1, PTK2B/FAK2), EGFR fam. (EGFR, ERBB2, ERBB3, ERBB4), FGFR fam. (FGFR1, FGFR2, FGFR2(N549H), FGFR3, FGFR4), FLT/VEGFR fam. (FLT1 (VEGFR1), FLT3, FLT3(D835Y), FLT4 (VEGFR3), KDR (VEGFR2)), IGFR/INSR fam. (IGF1R, INSRR (IRR), INSR), PDGFR fam. (PDGFRA, PDGFRB), ROR fam. (PDGFRA, PDGFRB), EPHA/B fam. (EPHA1, EPHA2, EPHA3, EPHA4, EPHA5, EPHA8, EPHB1, EPHB2, EPHB3, EPHB4), ALK fam. (ACVR1, ACVR1B, TGFBR1, TGFBR2, BMPR1B), AKT fam. (AKT1, AKT1(E17K), AKT2, AKT2(E17K), AKT3, AKT3(E17K), AKT3(G171R)), RAF fam. (ARAF, BRAF, RAF1 (cRAF)), AURK fam. (AURKA, AURKB, AURKC), CHEK fam. (CHEK1, CHEK2), PLK fam. (PLK1, PLK3), BRSK fam. (BRSK1, BRSK2), CAMK fam. (CAMK1, CAMK1beta, CAMK1delta, CAMK1gamma, CAMK2, CAMK2A, CAMK2B, CAMK2D, CAMK2G, CAMK4), CDK fam. (CDK1 (CDCl₂), CDK2, CDK3, CDK4, CDK5, CDK6, CDK7, CDK8, CDK9, CDK11), IKK fam. (CHUK (IKK alpha), IKBKB (IKK beta), IKBKE (IKK-E)), CLK fam. (CLK1, CLK2), CK fam. (CSNK1A1 (CK1A), CSNK1D (CK1D), CSNK2A1 (CK2A1), CSNK2A2 (CK2A2), CSNK2B (CK2B)), IRAK fam. (IRAK1, IRAK4), EIF2AK fam. (EIF2AK1, EIF2AK2, EIF2AK3 (PEK), EIF2AK4), GRK fam. (GRK3 (ADRBK2), GRK5), GSK3 fam. (GSK3A, GSK3B), ERK fam. (MAPK1 (ERK2), MAPK3 (ERK1)), JNK fam. (MAPK8 (JNK1), MAPK9 (JNK2), MAPK10 (JNK3)), p38 fan. (MAPK11 (p38b), MAPK12 (p38g), MAPK13 (p38d), MAPK14 (p38a)), MEK fam. (MAP2K1 (MEK1, MKK1, MAPKK1), MAP2K2 (MEK2, MKK2, MAPKK2)), MAP3K fam. (MAP3K1 (MEKK1, MAPKKK1), MAP3K7 (MEKK7, TAK1), MAP3K8 (COT), MAP3K14 (NIK), MAP3K17 (TAOK2, PSK1)), MAPKAPK fam. (MAPKAPK2 (MK2), MAPKAPK3 (MK3), MAPKAPK5 (MK5)), MARK fam. (MARK1, MARK2 (EMK1), MARK3 (CTAK1)), NEK fam. (NEK1, NEK2, NEK6, NEK7), NME fam. (NME1 (NM23, NDPK-A), NME2 (NM23B, NDPK-B), NME1-NME2), PAK fam. (PAK1, PAK2 (p21 (RAC1) activated kinase 2), PAK3, PAK4, PAK7), PIM fam. (PIM1, PIM2, PIM3), PKN fam. (PKN1 (PRK1), PKN2 (PRK2)), AMPKa fam. (PRKAA1 (AMPKa1), PRKAA2 (AMPKa2)), PKA fam. (PRKACA (PKA C-alpha), PRKACB (PKA C-beta), PRKACG (PKA C-gamma)), PKC fam. (PRKCA (PKC alpha, PKCA), PRKCB (PKC beta, PKCB), PRKCD (PKC delta, PKCD), PRKCE (PKC epsilon, PKCE), PRKCG (PKC gamma, PKCG), PRKCH (PKC eta, PKCL), PRKCI (PKC iota, PKCI), PRKCM (PKC mu, PKCM), PRKCN (PKC nu), PRKCQ (PKC theta), PRKCZ (PKC zeta)), PKD fam. (PRKD1 (PKD1, PKD), PRKD2 (PKD2), PRKD3 (PKD3)), PKG fam. (PRKG1 (PKG 1, subunits I-alpha & I-beta, PRKGR1, PRKG1=protein kinase, cGMP-dependent (PKG), type I), PRKG2 (PKG 2, PRKGR2, PRKG2=protein kinase, cGMP-dependent (PKG), type II)), ROCK fam. (ROCK,1 ROCK2), RPS6KA fam. (RPS6KA1 (RSK1, p90RSK), RPS6KA2 (RSK3, p90RSK2, S6K-alpha-2), RPS6KA3 (RSK2, p90RSK2, S6K-alpha-3), RPS6KA4 (MSK2, S6K-alpha-4), RPS6KA5 (MSK1), RPS6KA6 (RSK4, p90RSK6, S6K-alpha-6)), RPS6KB fam. (RPS6KB1 (S6K1, p70S6K, S6K-beta-1), RPS6KB2 (S6K2, p70S6Kb, S6K-beta-2)), SGK fam. (SGK1 (SGK), SGK2, SGK3 (SGKL)), and TSSK fam. (TSSK1, TSSK2, TSSK4).

In some embodiments, a kinase activity panel to identify a kinase signature that represents a kinase family or a specific kinase comprises biological sensor peptides that correspond to phosphorylated substrate regions from naturally occurring peptides that are phosphorylated by members of various kinase families, e.g., ABL, AKT, ERK, HER, p38, SFK, and TK kinase families. A kinase activity panel can, however, include a plurality of sensor peptides for any kinase of interest, including pseudo kinases and orphan kinases.

In some embodiments, a kinase activity panel in accordance with the invention comprises biological peptides that are phosphorylated by kinases enzymes that can include kinases evaluated in cancer cells and tumor cells, or other disease conditions, including BLK, BRK (PTK6), FGR, FRK, FYN, HCK, LCK, LYN, SRC, SRMS, YES1, ABL1, ABL2, EGFR, ErbB2, ErbB4, JAK2, CSK, AKT1, AKT2, AKT3, MAPK1 (ERK2), and/or MAPK14 (p38a) kinases. In some embodiments, a panel may comprises biological peptides that are phosphorylated by kinases found in cancer and tumor cells and may include biological sensor peptide fro one or more of PIMs, RPS6KBs, PAKs, PDPK1, GSK3B, PKCs, PKDs, PKAs and the like. In some embodiments, the panel comprises at least 3, or at least 4, biological peptides for each kinase; or at least 4 peptides for each of the kinase families. In some embodiments, the panel comprises 5, 6, 7, 8, 9, 10, 11, or 12 peptides for each kinase family represented in the panel. In some embodiments, the panel comprises 5, 6, 7, 8, 9, 10, 11, or 12 peptides for each kinase represented in the panel.

In some embodiments, a kinase panel in accordance with the invention comprises at least 75, at least 80, at least 90, at least 100, at least, 110 at least 120, at least 125, at least 130, at least, or at least 140, or at least 150, or 151 of the biological peptides comprise a sequence as shown in Table 1 of a peptide having a peptide ID as shown in FIG. 4. In some embodiments, the kinase activity panel further comprises at least 10, at least 15, at least 20, at least 25, or 27 mutated peptides having a mutation in the substrate region as shown in FIG. 4. In some embodiments, the mutated peptides have the sequence as shown in Table 1 for the peptide ID shown in FIG. 4. In some embodiments, the panel further comprises pre-phosphorylated reference peptides as mutated peptides.

In some embodiments, a kinase activity panel in accordance with the invention comprises 228 peptides as described in FIG. 4. The sequences of the peptides are provided in Table 1 for each peptide under the peptide probe ID number shown in FIG. 4. The panel comprises 151 biological peptides, 14 generic positive control peptides and 63 reference peptides, i.e., mutated peptides. The 14 generic positive control peptides are commonly uses as industry standards. The 63 reference peptides include 27 mutated peptide sequences, 31 pre-phosphorylated peptides and 5 peptides having random sequences. The phosphoresidue target is shown in FIG. 4, as is the origin of the peptide probe (i.e., the name of the biological substrate protein). The kinase enzyme associated with the biological peptides are also provided.

### Activity

Kinase activity can be evaluated using any number of assays, including, e.g., measuring ATP consumption; measuring consumption of any factor related to catalytic activity of an enzyme (e.g. GTP for GTPases instead of ATP for ATP-dependent kinases), measuring post-translational modification of any substrate molecule (e.g. the physical detection of the catalytic addition of a phosphorylation group onto a peptide; and the like). In some embodiments, ATP consumption is conveniently measured using an ATP consumption assay. An illustrative assay is detailed in the Examples under the Kinase activity assay section.

Activity pattern against various peptides in an assay panel can be assessed to determine kinase signature patterns as described herein. Activity can thus be used to assign the kinase activity of a sample undergoing evaluation to one or more kinase families. In some embodiments, kinase activity is assigned to one or more members of a kinase family. Assigning the activity of the kinase can be performed by comparing the activity to control patterns of activity of known kinases. The range of kinases activity of the test sample can vary from a high level of activity to little activity compared to the various control kinases.

In some embodiments, activity is evaluated as follows. Although this illustration of activity assessment uses ATP consumption to define activity, one of skill understands that similar analyses can be performed using endpoints other than ATP consumption to assess Activity. In an illustrative assay, ATP consumption is measured across a matrix comprising the kinase sensor peptides, e.g., the matrix may be wells or other containers that will hold the kinase assay contents. Again, although this is illustrated using a “well” as a container, other container may be used. Each well contains reagents/buffers, including the sensor peptide, ATP, and sample. In some embodiments, a kinase inhibitor may be included as a reagent component. A subset of control wells are also employed in which no peptide is present to assess basedline ATP consumption in the sample. ATP consumption measured across all experimental wells provides an activity signature of a sample. A first activity analysis is performed to determine ATP consumption per peptide. This provides an ‘agnostic’ view of the results. All ATP consumptions for a sample represent a ‘fingerprint’. These fingerprints can be compared between samples to determine how different samples are. One example is to use such fingerprints to compare cancer cell lines or tumor tissues (or even recombinant kinases). The results can then be interpreted to assess ATP consumption per kinase.

In the kinase activity screening panel, as explained above, some of the peptides are typically biological sensor peptides, i.e., they have naturally occurring amino acid sequences that are phosphorylated by a kinase enzyme. In some embodiments, a variant of such a peptide may be employed where the peptide may a minor change e.g., a conservative substitution, that does not influence kinase activity. Kinase activity obtained using the biological sensor peptides can then be used to deconvolute the agnostic peptide phosphorylation signatures to measure the activity of their respective kinases.

In some embodiments, some kinases may have only one biological sensor peptide associated with them in the assay. Some kinases may have two, some three, etc. More peptides per kinase typically provide for higher sensitivity, specificity and differentiability to identify kinases and their activity levels (i.e. ATP consumption measured and averaged across peptides that are biologically related to each kinase). In typical embodiments, a threshold of at least four biological peptides per kinase is employed to measure the activity of a given kinase, although alternative values, such as three biological peptides per kinase, may also be used.

For a given kinase, all ATP consumption per biological peptide of this kinase is included to measure the average ATP consumption (i.e. activity) for this kinase. Thus, all activities (ATP consumptions) per peptide can be useful to measure a kinase activity. Activity levels between knases within a sample can then be compared, as can activity levels between samples. Normalization is performed by creating a scale and using a threshold value. This is illustrated in the descriptions of FIGS. 1 and 2. Determination of signature patterns and use of a panel to characterizes a kinase in a sample to be evaluated can be determined as described in the Figures; and in the description of Figures, e.g., FIGS. 5, 7, 9, 12, 16, 18, 21, 24, and 26. For example, a sample obtained from a melanoma patient may be evaluated for phosphorylation signatures using a 228-petide phosphorylation panel as described herein, see, e.g., 4a and Table 1. Phosphorylation activity can be analyzed using unsupervised heriarchical clustering, principal component analysis, and dual significance threshold selections. Such an analysis can identify not only phosphosignatures that are indicative or particular kinases, or kinases families, but hyperactivity can be identified; and such associates can be associated with outcome.

In some embodiments, identification of overly active AKT1, PIM1, and RPS6Kb1 kinases as overly active in a melanoma sample is indicative of a poor prognosis (see, e.g., FIG. 23). In some embodiments, targeting RPS6KB or PIM1 in a patient having a BRAF^{V600E} melanoma in which RPS6KB1or PIM1 is elevated can provide an improved outcome compared to therapies that rarget other kinases.

### Samples

A “sample” as used herein comprises cells or is derived from cells, e.g., comprises cellular extracts, to be evaluated that can be obtained from any biological source, such as tissues, extracts, or cell cultures, including cells (e.g. tumor cells), cell lines, cell lysates. The sample can be obtained from animals, preferably mammals, most preferably humans. Samples can be from a single individual or in some embodiments, can be pooled prior to analysis. Samples may from any source may be evaluate dusing a panel of sensor peptides that provide the ability to simultaneous evaluated activities of different activities. Thus, a sample can be obtained from any prokaryote or eukaryote, including plants, yeast, bacteria, cyanobacteria, or any other biological source of interest.

Samples for analysis employing sensor peptides in accordance with the disclosure can be from any source for which it is desired to determine a kinase activity profile. In some embodiments, the sample is a mammalian sample, e.g., from primates (such as humans and non-human primates, e.g., apes, monkeys) or from other mammals, e.g., a bovine, ovine, porcine, equine, canine, feline, caprine, a murine, or other mammal.

In some embodiments, the sample is obtained from a patient that has a disease for which it is interest to assess kinase activity, where the sample comprises cells from a tissue that is affected by the disease. A “disease” refers to any disorder, disease, condition, syndrome or combination of manifestations or symptoms recognized or diagnosed as a disorder that may be correlated by a kinase activity profile. Illustrative disease include, but are not limited to, cancer, cardiovascular diseases including heart failure, hypertension and atherosclerosis, respiratory diseases, renal diseases, gastrointestinal diseases including inflammatory bowel diseases such as Crohn's disease and ulcerative colitis, hepatic, gallbladder and bile duct diseases, including hepatitis and cirrhosis, hematologic diseases, metabolic diseases, endocrine and reproductive diseases, including diabetes, bone and bone mineral metabolism diseases, immune system diseases including autoimmune diseases such as rheumatoid arthritis, lupus erythematosus, and other autoimmune diseases, musculoskeletal and connective tissue diseases, including including arthritis, achondroplasia infectious diseases and neurological diseases such as Alzheimer's disease, Huntington's disease and Parkinson's disease.

### Cancer

In some embodiments, a sample evaluated in accordance with the disclosure is a cancer. Cancer for which kinase activity profiles can be obtained include breast cancer, melanoma, colorectal cancer, lung cancer, ovarian cancer, uterine cancer, cervical cancer, prostate cancer, pancreatic cancer, bladder cancer, head and neck cancers, liver cancer, kidney cancer, brain cancer, including glioma and astrocytomas, thyroid cancer, laryngeal cancer, nasopharyngeal cancer, and oropharyngeal cancer; stomach cancer, and testicular cancer.

In some embodiments, the cancer is a hematological cancer, such as a lymphoma or leukemia, or multiple myeloma. Examples of leukemias include acute lymphoblastic leukemia, acute myeloid leukemia, chronic myeloid leukemia, hairy cell leukemia, acute nonlymphocytic leukemia, chronic lymphocytic leukemia, acute granulocytic leukemia, chronic granulocytic leukemia, and acute promyelocytic leukemia, among others. Examples of lymphomas include cutaneous T-cell lymphoma, Hodgkin's lymphoma, an non-Hodgkin's lymphoma.

In some embodiments, a sample comprising cancer cells is obtained from a patient who has been treated with a therapeutic agent, such as a chemotherapeutic agent, e.g., a protein kinase inhibitor.

In some embodiments, the analysis of kinase activity in cancer cells can be used to select a therapeutic agent that targets a kinase identified as being overactive in cancer cells.

The following examples are intended to illustrate, but not limit, the claimed invention. Thus, a panel of the invention may comprise a multiplicity of sensor peptides to detect activity for any kinases of interest and the reaction conditions and reagents for assessing activity can be modified as appropriate for evaluation of the kinase activity of interest.

### EXAMPLES

### Example 1. Peptide Multiplex Platform as a Robust Sensor of Kinases' Activity

Directly measuring the activity of kinases is the most proximal assessment of their functional status. This example describes the development of a high throughput kinase activity-mapping (HT-KAM) assay, whereby a compendium of biological peptide targets of kinase enzymes serves as combinatorial sensors of phospho-catalytic activity.

### Description of Experimental Results

### Peptide Sensing Platform to Monitor Phospho-Signatures

We sought to develop a high-throughput kinase activity-mapping (HT-KAM) assay, whereby a compendium of peptides serves as combinatorial sensors of the phospho-catalytic activity of kinase enzymes. To demonstrate our strategy, we synthesized a 228-peptide library (FIG. 4) that includes 151 biological 11-mer peptides corresponding to substrate protein regions variously phosphorylated by kinases involved in oncogenic processes³⁴. The library also includes 14 generic ‘positive control’ peptides commonly used as industry standards, and 63 reference peptides comprising 27 mutated, 31 pre-phosphorylated, and 5 random peptide sequences. A liquid dispensing instrument was programmed to aliquot peptide, sample, ATP, and buffer solutions in 384-well plates (FIG. 1a; Additional Methods section). Each well contains one peptide, and each plate simultaneously assesses the phospho-signature of one sample.

The description section below concentrates on the biological relevance and technical advance offered by our strategy. Analyses demonstrating repeatability, specificity and validation for recombinant kinases (FIG. 1; FIGS. 5-17), cell extracts (FIG. 2; FIGS. 18-21) and tissue extracts (FIG. 3; FIGS. 23-26) are briefly explained in this section, and more fully described in the Additional Methods section.

Multi-Peptide-Derived Phospho-Catalytic Signatures Distinguish Individual Kinases and their Enzymatic Subfamilies

The phospho-catalytic activity profile of 25 recombinant kinases was measured in presence of all 228-peptides (FIG. 1b; FIGS. 5-7). Inspection of kinases' activity across all peptides revealed that each kinase displayed a unique phosphorylation fingerprint (FIG. 1b). Particular family members were functionally distinguishable from genetically related kinases (e.g. BRK or SRMS vs. other SFK's; MAPK14 vs. MAPK1), and the activities of kinase isoforms or oncogenic variant were discernable (LYN A vs. LYN B; ABL1 vs. ABL1^{T315I}). Nevertheless, the principal factor clustering kinases was their family of origin (FIG. 1b, horizontal clustering; FIG. 1c, Pearson correlation grid).

We then examined how including a multiplicity of peptide sensors impacted the sensitivity and specificity of the assay for predicting the identity of an individual kinase. We computed Area Under the Curve (AUC) from repeated iteration of random peptide sampling. Sensitivity and specificity systematically improved when including an increasing number of peptides, while any single peptide performed poorly overall (example in FIG. 1d; FIGS. 8-9). For any given number of peptides, specific subsets performed significantly better than others. For instance, for HCK, the AUC derived from the specific combination of its 8 biological peptide targets was higher than most other 8-peptide combinations (FIG. 1d).

Next, we asked whether our system could find peptide sets that best differentiate a kinase from others. We compared all phospho-catalytic profiles of kinases using a dual significance threshold (p<0.05 for FDR-corrected t-test and Wilcoxon rank sum test). This revealed that a unique differential phospho-signature could be systematically assigned to every kinase (FIG. 1e; FIGS. 10-11). Optimal signatures included every type of peptide (biological, generic, reference) associated with both significantly high and significantly low phosphorylation activities. So, using a large spectrum of phospho-catalytic activity sensors is a highly sensitive reporting system to differentially identify a specific kinase/kinase family.

Biological Peptides are Effective Combinatorial Activity Sensors of their Kinase Enzymes

We then asked whether kinases preferentially phosphorylated their respective biological peptides. We found that kinases were significantly more capable of phosphorylating the great majority of their biological peptide targets than control pools of 63-reference, or 5-random, or 16-Y/S/T-free peptides (example of AKT1, MAPK1, JAK2 in FIG. 1f; box plots in FIG. 1g; complete dataset in FIGS. 12-13). Unsupervised clustering of kinases' activity using only biological peptides showed that biological peptides distinguished individual kinases, yet grouped them by functional relationships and kinase families (FIG. 1h). Biological peptides contributed most to kinases' differential phospho-signature (FIG. 1e,h; FIGS. 10-11) and highest measurable activity (FIG. 14). Computational analysis revealed that the phospho-catalytic signatures of kinases derived from their biological peptides outperformed generic peptides and provided excellent specificity and sensitivity (average AUC>0.9; FIG. 1d,i; FIG. 15).

To further evaluate the performance of biological peptides as sensors of kinases, we measured the effect of kinase inhibitors. The activity profiles of ABL1, ABL1^{T315I}, LYN A and AKT1 treated with dasatinib, imatinib and staurosporine showed that biological peptides effectively revealed the distinct drug-sensitivities of kinases (FIG. 1j-k; FIGS. 14b, 16). Statistical analyses determined that biological peptide sets systematically and significantly correlated with kinases' activity inhibition (FIGS. 14b, 16). Similar results were found when measuring the effects of SFK-inhibitor PP2 and SU6656 on FYN A, HCK, LCK, and SRC (FIG. 17). Altogether, these results show that the identity and activity of kinases can be measured using their respective biological peptides.

Identifying Druggable Kinases that Mediate Intrinsic Resistance to BRAF^{V600E}-Targeted Therapy

Providing a functional assay that identifies hyperactive, druggable kinases in cell culture models would be valuable to investigators. In the case of BRAF^{V600E} CRC, finding targeted therapies has been a biomedical challenge. RNA-interference screens performed in WiDr BRAF^{V600E} CRC cells originally found that parallel feedback activation of EGFR caused intrinsic resistance to vemurafenib (VEM)^{4,16}, but the limited response of patients treated with BRAF+EGFR combination therapy³⁷ underlines how crosstalk between signaling pathways often confounds genetic screen-drug response relationships. We applied our assay to explore whether other kinases drive such unresponsiveness to BRAF-therapy.

We used WiDr cells as a model system. WiDr treated with VEM displayed reduced MEK1 and ERK2 kinase activity, and increased EGFR activity (FIG. 2a; FIG. 18). This matched the reduction in phospho-MEK1/2 and ERK1/2 proteins, and increase in phospho-EGFR, which confirmed that the HT-KAM assay could functionally replicate what is anticipated from literature. We then concentrated on finding novel targets. Examination of kinase signatures revealed that AKT1 was overly active, while its downstream tumor suppressor kinase GSK3B was inactivated (FIG. 2b, left). Furthermore, the activity of PDPK1 and its downstream effector kinases SGK1, PRKCA, PKNs were increased (FIG. 2b, right). These changes in kinase activity were significant (FIG. 2b; FIGS. 18-19) and confirmed by immuno-detection (FIG. 2c). Such specific changes are cancer-promoting processes implicated in cell cycle, survival and metabolism, which suggests that drug resistance can be functionally defined by our assay as the coordinated re-programing of multiple signaling pathways.

To assess the role of these kinases as mediators of resistance to VEM, we tested the response of WiDr to drug combinations in cell survival assays. Besides AKT1, strong synergy was observed when BRAF^{V600E}-targeting was paired with inhibitors for PDPK1 and PRKCA (FIG. 2d). Results were confirmed using other BRAF^{V600E} CRC cell lines and inhibitors (FIG. 20). This demonstrates that our strategy can serve as a discovery platform to predict differences in kinase activity and provide a rational design for new combination therapies.

The Phospho-Catalytic Signatures of Cancer Cells Reveal their Specific Kinase Dependencies

Next, we asked whether our approach could be used to survey the activity of kinases across different cancer cell lines. FIG. 2e-f and FIGS. 21-22a,b show that cancer cell lines could be systematically distinguished based on either their 228-peptide phosphorylation profiles, or their kinase activity signatures. We then evaluated whether the differential kinase activities of different cells could foretell their drug sensitivity (FIG. 22c-e), and specifically asked whether HT-KAM could identify kinases that functionally distinguish BRAF^{V600E} CRC from BRAF^{V600E} MEL cells (WiDr vs. A375; FIG. 2g-h). Graphs in FIG. 2g compare kinase activity (y-axis) to GI50 concentration for individual drugs (x-axis). Data for the 10 kinases and 14 inhibitors we tested, are compiled in FIG. 2h. Results showed that the higher the activity of a kinase, the more a cell line was susceptible to respond to a matching drug. For instance, CDKs and MEKs were significantly more catalytically active in A375 than in WiDr, and A375 cells respond ed to much lower concentrations of the related drugs dinaciclib and trametinib than WiDr did. So, BRAF^{V600E} cells of different tumor origins inherently relied on distinct kinase dependencies that were predictive of their drug sensitivities. Altogether, results in FIG. 2f-h and FIG. 22 indicate that our platform provides a pragmatic solution to find active, druggable kinases in cell culture models.

### Mapping the Phospho-Catalytic Signatures of Patients' Melanomas

Identifying which kinases are overly active in patients' tumors would be of high clinical value. Malignant melanoma is a disease ultimately refractory to most current forms of therapy, including BRAF/MEK/ERK inhibitors used to treat metastatic BRAF^{V600E} tumors³⁸⁻⁴¹. We thus evaluated the potential utility of our assay to map the phospho-catalytic signatures of patients' melanomas.

Nine surgically excised, fresh-frozen tumors from melanoma patients (FIG. 3a; FIG. 23) were tested in four independent HT-KAM technical replicates. The 228-peptide phosphorylation signatures were analyzed using unsupervised hierarchical clustering (FIG. 3b), principal component analysis (FIG. 24), and dual significance threshold selection (FIG. 25). We found that the phospho-fingerprints of tumors were highly robust signatures that strongly associated with outcome (FIG. 3b, black squares; FIGS. 24, 25). The group of signatures that retrospectively predicted poor outcome included BRAF^{V600E} tumors that did not respond to VEM treatment (FIG. 3b, red squares; FIGS. 24, 25b-e).

Next, we asked whether peptide phosphorylation profiles could reveal the hyperactive kinases of poor outcome tumors. FIG. 3c and FIG. 26a-c show that kinase activity signatures established from biological peptides and compared across tumors, were associated with outcome. Enrichment analysis using the most significantly and differentially phosphorylated biological peptides in poor outcome patients, determined that PIM, RPS6KB and AKT kinases were most active (FIG. 3d; FIGS. 26d-e, 27), and highest in VEM-resistant melanomas (FIG. 3c, arrows; FIG. 3e). GSK3B was significantly downregulated in these tumors (FIG. 3c,e). These hyperactive kinases suggest new vulnerabilities that may be exploitable in the clinic.

Translating Kinase Hits into New Therapeutic Opportunities for BRAFV600E Melanoma Treatment

Based on these patient tumor kinase activity profiles, we assessed the growth response of A375 and Sk-Mel-28 cells to kinase-targeting drug combinations. FIG. 3f-h and FIG. 28 show that inhibitors of PIM, RPS6KB or AKT significantly potentiated the anti-cancer effects of BRAF inhibition, and outperformed MTOR- or MEK-inhibitors whose effects in patients are variable and transient. Cells exogenously expressing constitutively active AKT1 remained sensitive to RPS6KB- or PIM-targeting (FIG. 29), suggesting that these signaling hubs can function independently and represent suitable alternative targets to alleviate therapeutic resistance. GSK3B inhibition antagonized VEM effects, thus mimicking a loss of tumor suppressive function by promoting resistance to BRAF-therapy. These results support observations we made in patient tumors (FIG. 3c-e), where unresponsiveness to BRAF^{V600E}-therapy was accompanied with the coordinated inactivation of GSK3B and activation of PIM, RPS6KB and AKT kinases.

Finally, we used tumor cells isolated from a patient-derived xenograft (PDX) established from a BRAF^{V600E} melanoma patient refractory to VEM 42, and that maintained high levels of phospho-RPS6KB1 (FIG. 3i; FIG. 30). Colony formation showed these tumor cells were particularly sensitive to RPS6KB inhibition (FIG. 3i). So, the activation of RPS6KB is a confirmed vulnerability that can be targeted to restore therapeutic sensitivity in BRAF therapy-resistant melanomas.

### Discussion of Experimental Results Described Above

A key to successful therapy is the identification of critical aberrant signaling networks whose inhibition would result in system failure of diseased cells. This example demonstrates the use of an innovative proteomic approach to identify specific kinase vulnerabilities that lie within the proto-oncogenic phospho-circuits of cancer cells and tumor tissues.

We developed a system that relies on collections of peptides to directly monitor the phospho-catalytic signatures of biological samples. Our strategy provides access to a vast, untapped resource of meaningful measurements, whether readouts are interpreted irrespective of which enzymes phosphorylate which probes, or analyzed to convert global phospho-signatures into functional profiles of kinase activities.

In-depth computational analyses and systematic drug targeting of kinases established the advantages of our system to gain biological insights into phospho-signaling circuits. The combination of single phospho-activities measured across peptide sensors collectively distinguished the activity of different kinases within or between samples of simple or complex composition (purified kinases, cells, tissues). Particular subsets of peptides-especially kinases' biological targets-provided superior sensitivity, specificity and discernibility over any single probe-derived measurement. The identity and activity of kinases across a broad range of kinase families could be simultaneously assessed from their subset of biological peptides in various cancer cells and tumor tissues.

Based on our analyses, the differential spectrum of low-to-high phospho-catalytic activities measured across peptides predicts that expanding the peptide library beyond the current 228 peptides will enable mapping many more kinases with even finer accuracy. It will also be possible to build ‘fit-for-purpose kits’ relying on narrow sets of best-predictor peptides customized to monitor the activity of particular kinases for research or diagnostic purposes. The HT-KAM strategy is a versatile platform adaptable to users' needs (e.g. interchangeable peptide library, or assay conditions), and practical to both laboratory research and clinical settings.

Mechanisms of drug resistance are the cornerstone of therapeutic failure. We established that peptide phospho-signatures of cancer cells could be deconvoluted to identify hyperactive, druggable kinases. We showed that the intrinsic and adaptive kinase dependencies of different BRAF^{V600E} cancers were distinctive and predictive of their drug-sensitivity to single or combinatorial targeted therapies. The newly revealed kinase vulnerabilities of VEM-resistance in BRAF^{V600E} CRC cells included signaling pathways orchestrated by PDPK1, PRKCA, SGK1 and GSK3B. In melanoma, PIM1 and RPS6KB1 were identified as new druggable vulnerabilities predictive of poor outcome in BRAF^{V600E} patients. Some of these susceptibilities are new therapeutic alternatives that could be tested in the clinic. Since kinase circuits are likely cell/patient specific, it will be important to profile the kinome signatures of individual patients' tumors to personalize medical treatments.

Our approach effectively identified kinase targets beyond those previously found by synthetic lethality genetic dropout screens in same model systems. While shRNA approaches focus more on individual genetic dependencies, our assay allows capturing the functional fingerprint of kinases in their native state, without requiring exogenous interventions that may alter the dynamics of signaling circuits. Furthermore, large scale gene expression or mutation analyses would not identify the kinase targets we found because no evident genetic alteration are reported for these genes in cell lines or patient tumors (e.g. RPS6KB1 or PIM1 in MEL; PRKCA or PDPK1 in CRC)^{18,34,42,43}. As well, the results of considerable genomic study efforts suggest that, for many cancers including CRC, therapeutic resistance is likely not driven by individual genetic dependencies or caused by some dominant driver mutations. Therefore, functional proteomic platforms designed to detect the activity of kinases—and eventually the functionality of the whole kinome—such as HT-KAM, are needed to start elaborating higher-order functional maps of signaling networks that can directly pinpoint actionable vulnerabilities of tumors.

How to choose and pair limitless combinations of drugs is a pressing need for pharmaceutical industries and physicians who grapple with treatment resistance in patients⁴⁴⁻⁴⁶. Our platform provides a new rational design to help prioritize drug combinations and maximize likelihood of success. Furthermore, the phospho-peptide signatures uncovered in our study represent a new parameter that has the potential to be configured into diagnostic tests. For instance, BRAF^{V600E} melanoma patients who retrospectively displayed phospho-signatures indicative of aggressive disease, may have benefited from targeting BRAF+RPS6KB1 or PIM1, instead of standard therapies on which patients almost inevitably relapse.

The fact that our assay identifies multiple kinases as a cause of drug-resistance defies the usual scenario of trying to map a highly specific feedback loop that ends up describing a ‘unique’ pathway that mediates ‘all’ of the observed resistance. Instead, our results argue that resistance can result from a combination of pathways that are upregulated, working in concert, and interdependent on each other, such that it requires their coordinated signaling activities to drive the resistance. As such, a finite number of key cooperative dependencies can be identified, thus offering a highly selective choice of relevant targets to explore. Such herd-like mode of resistance can only be discovered by the kind of mass-scale functional proteomic approach we developed.

The combinatorial peptide sensing system described herein is thus a new, effective way to capture the functionality of kinases in cells and tissues. This modular strategy addresses a central issue in the bio-medical field, and could play an integral role in improving research productivity and guiding therapeutic decisions. Mapping the phospho-catalytic signatures of diseases innovates the molecular exploration of signaling networks, and supports the discovery of new actionable dependencies for precision medicine.

### REFERENCES CITED BY NUMBER IN ABOVE EXPERIMENTAL RESULTS AND DISCUSSION. FIGURE DESCRIPTIONS FOR FIGS. 1-3. BACKGROUND OF THE INVENTION SECTION. AND FIRST PARAGRAPH OF BRIEF SUMMARY OF ASPECTS OF THE DISCLOSURE SECTION

1. Petricoin, E. F., Zoon, K. C., Kohn, E. C., Barrett, J. C. & Liotta, L. A. Clinical proteomics: translating benchside promise into bedside reality. Nature reviews. Drug discovery 1, 683-695 (2002).
2. Blume-Jensen, P. & Hunter, T. Oncogenic kinase signalling. Nature 411, 355-365 (2001).
3. Poulikakos, P. I., Zhang, C., Bollag, G., Shokat, K. M. & Rosen, N. RAF inhibitors transactivate RAF dimers and ERK signalling in cells with wild-type BRAF. Nature 464, 427-430 (2010).
4. Prahallad, A., et al. Unresponsiveness of colon cancer to BRAF(V600E) inhibition through feedback activation of EGFR. Nature 483, 100-103 (2012).
5. Duncan, J. S., et al. Dynamic reprogramming of the kinome in response to targeted MEK inhibition in triple-negative breast cancer. Cell 149, 307-321 (2012).
6. Sos, M. L., et al. Oncogene mimicry as a mechanism of primary resistance to BRAF inhibitors. Cell reports 8, 1037-1048 (2014).
7. Johannessen, C. M., et al. COT drives resistance to RAF inhibition through MAP kinase pathway reactivation. Nature 468, 968-972 (2010).
8. Janne, P. A., Gray, N. & Settleman, J. Factors underlying sensitivity of cancers to small-molecule kinase inhibitors. Nature reviews. Drug discovery 8, 709-723 (2009).
9. Fleuren, E. D., Zhang, L., Wu, J. & Daly, R. J. The kinome ‘at large’ in cancer. Nature reviews. Cancer 16, 83-98 (2016).
10. Johnson, S. A. & Hunter, T. Kinomics: methods for deciphering the kinome. Nature methods 2, 17-25 (2005).
11. Krogan, N.J., Lippman, S., Agard, D. A., Ashworth, A. & Ideker, T. The Cancer Cell Map Initiative: Defining the Hallmark Networks of Cancer. Molecular cell 58, 690-698 (2015).
12. Hoadley, K. A., et al. Multiplatform analysis of 12 cancer types reveals molecular classification within and across tissues of origin. Cell 158, 929-944 (2014).
13. Akbani, R., et al. A pan-cancer proteomic perspective on The Cancer Genome Atlas. Nature communications 5, 3887 (2014).
14. Chandarlapaty, S., et al. AKT inhibition relieves feedback suppression of receptor tyrosine kinase expression and activity. Cancer cell 19, 58-71 (2011).
15. Coppe, J. P., et al. Senescence-associated secretory phenotypes reveal cell-nonautonomous functions of oncogenic RAS and the p53 tumor suppressor. PLoS Biol 6, 2853-2868 (2008).
16. Huttlin, E. L., et al. The BioPlex Network: A Systematic Exploration of the Human Interactome. Cell 162, 425-440 (2015).
17. Rikova, K., et al. Global survey of phosphotyrosine signaling identifies oncogenic kinases in lung cancer. Cell 131, 1190-1203 (2007).
18. Zhang, B., et al. Proteogenomic characterization of human colon and rectal cancer. Nature 513, 382-387 (2014).
19. Uhlen, M., et al. Proteomics. Tissue-based map of the human proteome. Science 347, 1260419 (2015).
20. Drake, J. M., et al. Phosphoproteome Integration Reveals Patient-Specific Networks in Prostate Cancer. Cell 166, 1041-1054 (2016).
21. Sharma, K., et al. Ultradeep human phosphoproteome reveals a distinct regulatory nature of Tyr and Ser/Thr-based signaling. Cell reports 8, 1583-1594 (2014).
22. Bantscheff, M., et al. Quantitative chemical proteomics reveals mechanisms of action of clinical ABL kinase inhibitors. Nature biotechnology 25, 1035-1044 (2007).
23. Daub, H., et al. Kinase-selective enrichment enables quantitative phosphoproteomics of the kinome across the cell cycle. Molecular cell 31, 438-448 (2008).
24. Kubota, K., et al. Sensitive multiplexed analysis of kinase activities and activity-based kinase identification. Nature biotechnology 27, 933-940 (2009).
25. Nomura, D. K., Dix, M. M. & Cravatt, B. F. Activity-based protein profiling for biochemical pathway discovery in cancer. Nature reviews. Cancer 10, 630-638 (2010).
26. Ren, W., Damayanti, N. P., Wang, X. & Irudayaraj, J. M. Kinase phosphorylation monitoring with i-motif DNA cross-linked SERS probes. Chem Commun (Camb) 52, 410-413 (2016).
27. Anastassiadis, T., Deacon, S. W., Devarajan, K., Ma, H. & Peterson, J. R. Comprehensive assay of kinase catalytic activity reveals features of kinase inhibitor selectivity. Nature biotechnology 29, 1039-1045 (2011).
28. Houseman, B. T., Huh, J. H., Kron, S. J. & Mrksich, M. Peptide chips for the quantitative evaluation of protein kinase activity. Nature biotechnology 20, 270-274 (2002).
29. Karaman, M. W., et al. A quantitative analysis of kinase inhibitor selectivity. Nature biotechnology 26, 127-132 (2008).
30. Fang, C., et al. Integrated microfluidic and imaging platform for a kinase activity radioassay to analyze minute patient cancer samples. Cancer research 70, 8299-8308 (2010).
31. Li, X., et al. The reverse in-gel kinase assay to profile physiological kinase substrates. Nature methods 4, 957-962 (2007).
32. Wu, J., Barbero, R., Vajjhala, S. & O'Connor, S. D. Real-time analysis of enzyme kinetics via micro parallel liquid chromatography. Assay Drug Dev Technol 4, 653-660 (2006).
33. Sanz, A., et al. Analysis of Jak2 catalytic function by peptide microarrays: the role of the JH2 domain and V617F mutation. PloS one 6, e18522 (2011).
34. Olow, A., et al. An Atlas of the Human Kinome Reveals the Mutational Landscape Underlying Dysregulated Phosphorylation Cascades in Cancer. Cancer research (2016).
35. Hornbeck, P. V., et al. PhosphoSitePlus, 2014: mutations, PTMs and recalibrations. Nucleic acids research 43, D512-520 (2015).
36. Corcoran, R. B., et al. EGFR-mediated re-activation of MAPK signaling contributes to insensitivity of BRAF mutant colorectal cancers to RAF inhibition with vemurafenib. Cancer discovery 2, 227-235 (2012).
37. van Geel, R. M., Beijnen, J. H., Bernards, R. & Schellens, J. H. Treatment Individualization in Colorectal Cancer. Curr Colorectal Cancer Rep 11, 335-344 (2015).
38. Flaherty, K. T., et al. Combined BRAF and MEK inhibition in melanoma with BRAF V600 mutations. The New England journal of medicine 367, 1694-1703 (2012).
39. Larkin, J., et al. Combined vemurafenib and cobimetinib in BRAF-mutated melanoma. The New England journal of medicine 371, 1867-1876 (2014).
40. Van Allen, E. M., et al. The genetic landscape of clinical resistance to RAF inhibition in metastatic melanoma. Cancer discovery 4, 94-109 (2014).
41. Wagle, N., et al. MAP kinase pathway alterations in BRAF-mutant melanoma patients with acquired resistance to combined RAF/MEK inhibition. Cancer discovery 4, 61-68 (2014).
42. Kemper, K., et al. BRAF(V600E) Kinase Domain Duplication Identified in Therapy-Refractory Melanoma Patient-Derived Xenografts. Cell reports 16, 263-277 (2016).
43. Cancer Genome Atlas, N. Genomic Classification of Cutaneous Melanoma. Cell 161, 1681-1696 (2015).
44. Bernards, R. A missing link in genotype-directed cancer therapy. Cell 151, 465-468 (2012).
45. Cohen, R. L. & Settleman, J. From cancer genomics to precision oncology-tissue's still an issue. Cell 157, 1509-1514 (2014).
46. Holohan, C., Van Schaeybroeck, S., Longley, D. B. & Johnston, P. G. Cancer drug resistance: an evolving paradigm. Nature reviews. Cancer 13, 714-726 (2013).
47. Brunen, D., et al. TGF-beta: an emerging player in drug resistance. Cell Cycle 12, 2960-2968 (2013).
48. Foucquier, J. & Guedj, M. Analysis of drug combinations: current methodological landscape. Pharmacol Res Perspect 3, e00149 (2015).

### Additional Methods-Results are Summarized in the Desriptions of the Figures

Kinase activity assay. The phospho-catalytic signature of samples was established from simultaneously occurring ATP-consumption tests measured in presence of peptides that are experimentally isolated from each other in multi-well plates. Assays were run in 384 well-plates (solid white flat bottom plates; Corning, cat. #3570), where each experimental well contained one kind of peptide. The final 8 uL reaction mixtures per well contained the following final concentrations of reagents: (i) kinase assay buffer (KaBIx prepared daily and diluted in ddH₂O from a 10× stock solution of 25 mM Tris-HCl (pH7.5), 10 mM MgCl₂, 0.1 mM Na3VO4, 5 mM O-glycerophosphate, 2 mM dithiothreitol (DTT); or purchased from Cell Signaling cat. #9802), (ii) 250 nM ATP (prepared from a 10 mM stock solution of adenosine-5′-triphosphate in ddH₂O, and diluted daily with KaB1x; Cell Signaling cat. #9804), (iii) 200 ug/mL 11-mer peptide (lyophilized stocks originally prepared as 1 mg/mL in KaBIx, 5% DMSO), and (iv) samples typically made of either 5 ng/uL recombinant kinase enzyme protein or 10 ug/mL protein extract from cell or tissue lysates (see below for protocol) that were kept on ice and diluted in KaB1x <30 min before experimental testing. Controls with no-ATP, or no-peptide, or no-sample as well as ATP standards, were run side-by-side within each 384-well plate.

High-throughput liquid dispensing of all reagents was achieved using the Biomek® FX Laboratory Automation Workstation from Beckman Coulter (hosted by the Center for Advanced Technologies, UCSF), and was programmed to specifically address the dispensing requirements of the assay (timing, sequence, tip-touch location/height/depth, etc). Accurate dispensing was thoroughly and regularly validated. All reagents were kept on ice and plates on cold blocks (VWR/BioCision; COOLRACK XT PCR96 cat. #89239-498; COOLSINK XT 96F cat. #89239-504) until enzymatic reactions were started. For all intermediary steps over the course of the assay (i.e. buffer and sample preparation, dispensing, etc), we used micro-centrifuge tubes (Costar; cat. #3621) and clear 96-well PCR-plates (VWR; cat. #83007-374).

Once the dispensing of reaction mixtures was completed, 384-well reaction plates were typically incubated for 30 min at 30 degC. After enzymatic reactions were completed, the final detection step used Kinase-Glo revealing reagent (Promega; cat. #V3772; dispensed using Biomek automated workstation), which stops the activity of kinase enzymes and produces a luminescent signal directly correlated with the amount of remaining ATP in samples over a broad range of ATP concentrations (repeatability and accuracy of the ATP-dependent luminescence assay measurements were tested and validated over 5 logs of ATP concentration; R2>0.99). Luminescence data are inversely correlated with the amount of kinase activity. Luminescence was measured using the Synergy 2 Multi-Mode Microplate Reader from BioTek, and occasionally the Molecular Devices Analyst AD Microplate Reader from McKinley Scientific.

Peptide sensors. 11-mer amino acid sequences were made-to-order and mass synthesized by GenScript at >95% purity. The 228-peptide library included 151 biological peptides, 14 generic positive control peptides, and 63 reference peptides that include 27 mutated (Tyrosine (Y)/Serine (S)/Threonine (T)→Glycine (G)) and 31 pre-phosphorylated (Y/S/T→pY/pS/pT) peptides, and 5 random peptide sequences (FIG. 4 provides peptide sequence details and connectivity between peptides and kinases). Biological peptides correspond to phosphorylatable amino acid regions of substrate protein identified from literature and curated in resources such as PhosphoAtlas¹ (FIG. 4c-d) or PhosphoSitePlus². Each generic positive control peptide corresponds to a kinase activity reporting probe commonly used in single-peptide assays, as available/advertised from literature. Some of the generic positive control peptides were purchased from SignalChem (e.g. Abltide, cat. #A02-58; Poly (4:1 Glu, Tyr) peptide, cat #P61-58).

Recombinant kinases. The following purified, recombinant kinase enzymes were purchased from SignalChem: ABL1/c-ABL (cat. #A03-18H), ABL1^{T315I} (cat. #A03-12DG), ABL2/ARG (cat. #A04-11H), AKT1/PKB/RAC (cat. #A16-10G), AKT2 (cat. #A17-10G), AKT3 (cat. #A18-10G), BLK (cat. #B02-10G), BRK/PTK6 (cat. #P94-10G), CSK (cat #C63-10G), EGFR/HER1 (cat #E10-11G), ErbB2/HER2/NEU (cat #E27-11G), ErbB4/HER4 (cat. #E29-11G), ERK2/MAPK1/p42 (cat. #M28-10G), FGR (cat. #F10-10G), FRK (cat. #F14-11G), FYN isoforms A (cat. #F15-10G), FYN isoform C (inactive; cat. #F15-14G-20), HCK (cat. #H02-11G), JAK2 (cat. #J02-11H), LCK (cat. #L03-10G), LYN isoform A (cat. #L13-18G), LYN isoform B (cat. #L13-10G), p38a/MAPK14 (cat. #M39-10BG), SRC/c-SRC (cat. #S19-18G), SRM/SRMS (cat. #S20-11G), YES/YES1 (cat. #Y01-10G). The same concentration of every kinase was used in all experiments (FIG. 1, FIGS. 5-17).

Kinase inhibitors. The following 46 inhibitors were used in biochemical assays or cell culture. Inhibitors purchased from Selleck Chemicals are: AT13148 (cat. #S7563), AZD1208 (cat. #S7104), AZD7762 (cat. #S1532), BAY-61-3606 (cat. #S7006), BI-D1870 (cat. #S2843), bosutinib/SKI-606 (cat. #S1014), CI-1040/PD184352 (cat. #S1020), CHIR-99021 (cat. #S1263), dasatinib (cat. #S1021), dinaciclib/SCH-727965 (cat. #S2768), everolimus/RAD001 (cat. #S1120), fedratinib/SAR302503 (cat. #S2736), gefitinib/ZD-1839 (cat. #S1025), Go6983 (cat. #S2911), GSK2334470 (cat. #S7087), H89 (cat. #S1582), imatinib (cat. #S1026), IPA-3 (cat. #S7093), JNK Inhibitor VIII (cat. #S4901), LFM-A13 (cat. #S7734), LY2584702 (cat. #S7704), MK2206 (cat. #S1078), nilotinib/AMN-107 (cat. #S1033), NPK76-ii-72-1 (cat. #S), OSU-03012 (cat. #S1106), pelitinib/EKB-569 (cat. #S1392), PLX-4720 (cat. #S1152), PF-4708671 (cat. #S2163), ponatinib/AP24534 (cat. #S1490), RO-3306 (cat. #S7747), ruxolitinib/INCB018424 (cat. #S1378), saracatinib/AZD0530 (cat. #S1006), selumetinib/AZD6244 (cat. #S1008), sotrastaurin (cat. #S2791), TAK-715 (cat. #S2928), trametinib/GSK1120212 (cat. #S2673), vemurafenib/PLX4032 (cat. #S1267), VX-702 (cat. #S6005), 1-Azakenpaullone (cat. #S7193). Inhibitors obtained from other companies are AS601245 (Cayman; cat. #17542), bryostatin 1 (Sigma-Aldrich; cat. #B7431), PP2 (Invitrogen; cat. #PHZ1223), PP3 (Tocris; cat. #2794), SL 0101-1 (Tocris; cat. #2250), staurosporine (Sigma-Aldrich; cat. #S4400), SU6656 (EMD-CalBiochem; cat #572635). Conditions of use are indicated in the text.

Cell culture. The 23 cancer cell lines used in this study were purchased from ATCC or provided by the laboratories of Drs. R. Bernards, S. Ortiz-Urda, M. Bissell, or F. McCormick (colorectal: WiDr, HT29, SK-CO-1, HCT-116, RKO-1; melanoma: A375, Sk-Mel-28, Mel888, MM485, Sk-Mel-2; lung: H1755, H3122, PC9; breast: AU565, HCC70, MCF7, MDA-MB-231, MDA-MB-436, T47D, HMT-3522 S1, HMT-3522 T4; prostate: PC3; thyroid: 8505C). Cells were cultured following ATCC's instructions or as previously described 3. Information regarding primary melanoma cell lines derived from therapy-resistant BRAF^{V600E}-melanoma patient-derived xenografts (PDXs) are previously described^{4,5}.

To assess the growth/survival response of cell lines to single or combinatorial drug treatments in FIGS. 2-3 and FIGS. 20, 22, 28, 29, we used CellTiter-Glo cell viability assay (Promega; cat #G7571). Cell culture and luminescence readouts were performed in 96- and 384-well plates after 3-day treatments. Drug treatment conditions are described in the text. The effects of drug combinations on cell growth were assessed by calculating drug interaction (D.I.; two-way ANOVA using Prism or Sigma Plot software) and combination index (C.I.; following either the Loewe Additivity⁶⁻⁹ or Bliss Independence models¹⁰⁻⁴²). IC50 and G150 correspond to the concentration of a given drug that causes 50% inhibition of kinase activity (IC50) or cell growth (GI50). We systematically tested the effects of drugs in both regular and low serum culture conditions (i.e. 5% and 0.25% FBS media). To quantitate cell death in FIG. 3 and FIGS. 28-29, FACS analysis of nuclear degradation was performed as previously described¹³. Colony formation assay in FIG. 3 and FIG. 30 were performed as previously described⁵.

To test the responses of WiDr cells treated with VEM in FIG. 2 and FIGS. 18-20, cells were plated in medium containing 10% FBS 24 h prior to being washed with serum-free medium, and cultured for 24 h in medium containing 0.1% serum 3. After low serum incubation, cells were treated with drugs for 30 min and stimulated by 10% FBS. After 8 h, 85% confluent cells were washed in PBS, then lyzed, and supernatants stored at −80 degC. In cases where experiments happened at the Netherlands Cancer Institute, samples were shipped on dry-ice to the University of California at San Francisco.

Preparing protein lysates from cell lines for kinase assay. To measure the phospho-catalytic activity of cancer cell lines (FIG. 2 and FIGS. 18, 19, 21, 22), cultured cells were lyzed for 5 min in ice-cold cell lysis buffer. Freshly prepared lysis buffer (1 mL per 5.10\hat{ }6 cells) contained non-denaturing Cell Lysis Buffer 1× (diluted in ddH₂O from 10× stock of 20 mM Tris-HCl (pH7.5), 150 mM NaCl, 1 mM Na2EDTA, 1 mM EGTA, 1% Triton, 2.5 mM sodium pyrophosphate, 1 mM b-glycerophosphate, 1 mM Na3V04, 1 ug/mL leupeptin; or purchased from Cell Signaling, cat. #9803), complemented with 1× Halt Protease & Phosphatase (ThermoScientific cat. #1861281, which contains inhibitors of Ser/Thr-phosphatases and Tyr-phosphatases). Scraped off lysates were then spun down at 14,000 rpm for 15 min, and supernatants stored at −80 degC. Protein and ATP concentrations were quantified. Since every HT-KAM assay plate includes 14 wells with sample alone (i.e. without peptide; see (FIG. 4f), internal controls for ATP levels were systematically available for all assays/samples (see FIGS. 18, 21, 22).

Immuno-detection and antibodies. Cell lysates were resolved by SDS gel electrophoresis (gels from BioRad), followed by immuno-blotting as previously described^{3,5} and following manufacturer's instructions. The following primary antibodies and phospho-antibodies were used. Antibodies to detect ATK1/2/3 (cat. #4691), AKT1/2 pS473 (cat. #4060), AKT1/2 pT308 (cat. #2965), EGFR (cat. #4267 and 3771), EGFR pY1068 (cat. #3777 and 2234), ERK1/2 (i.e. MAPK1/ERK2/p44 and MAPK3/ERK1/p42; cat. #4695), ERK1/2 pT202/Y204 (cat. #4370), GSK3B (cat. #9315), GSK3B pS9 (cat. #9323), MAPK14/p38a (cat. #8690), MAPK14/p38a pT180/pY182 (cat. #9215), MEK1/2 (i.e. MAP2K1 and MAP2K2; cat. #4694), MEK1/2 pS217/221 (cat. #9121), MTOR (cat. #2972), MTOR pS2448 (cat. #2971), PIM1 (cat. #3247), PDPK1/PDK1 (cat. #3062), PDPK1/PDK1 pS241 (cat. #3438), PKN1/PRK1 pT774 and PKN2/PRK2 pT816 (cat. #2611), PRKCA/PKCa (cat. #2056), PRKCA/PKCa pT514 (cat. #9379), RPS6KA1/p90RSK1 (cat. #8408), RPS6KA1/p90RSK1 pT353 (cat. #8753), RPS6KB1/p70S6K1 (cat. #2708), RPS6KB1 pT389 (cat. #9234), RPS6KB1/p70S6K1 pT421/pS424 (cat. #9204), SGK1 (cat. #3272), SGK1 pS78 (cat. #5599), were from Cell Signaling. Antibodies to detect ERK1 (C-16), ERK2 (C-14), ERK1/2 pT202/pY204 (E-4), HSP90 (cat. #sc-7947) were from SantaCruz. A mixture of ERK1 and ERK2 antibodies was used for detection of total ERK 3. Dilutions followed manufacturers' instructions.

Tumor specimens from melanoma patients. Clinical details regarding patients are available in FIG. 23a. Patient samples were collected at The Rudolfstiftung Hospital, Vienna Austria, and the University of California San Francisco, Calif. USA, under the IRB #13-204-VK and 12-09483, respectively. Tumor tissue not needed for diagnostic purposes were collected intraoperatively, macroscopically dissected and flash frozen. A small piece of tumor tissue was O. C. T.-embedded, sectioned, H&E stained and analyzed to ensure >80% tumor cell content in tumor tissue samples.

Preparing protein lysates from tumor tissues for kinase assay. Flash frozen melanoma tissue specimens were pulverized using BioSpec 59012MS. Protein extracts from powdered samples were prepared following the lysis protocol used for cultured cells, i.e. lyzed for 5 min in ice-cold non-denaturing CLBIx diluted in ddH₂O from 10× stock of 20 mM Tris-HCl (pH7.5), 150 mM NaCl, 1 mM Na2EDTA, 1 mM EGTA, 1% Triton, 2.5 mM sodium pyrophosphate, 1 mM b-glycerophosphate, 1 mM Na3VO4, 1 ug/mL leupeptin, complemented with 1× Halt Protease & Phosphatase (containing Ser/Thr- and Tyr-phosphatases inhibitors), then spun down at 14,000 rpm for 15 min, and supernatants collected. All samples were stored at −80 degC. Internal controls for ATP levels in peptide-free wells were systematically measured (see FIG. 23b). The total amount of tumor tissue sample necessary to profile the activity of kinases across 384 well/plates, ranged from 20 ug to 30 ug (which is less than the typical 100 ug collected per core biopsy).

TCGA data analysis. mRNA data from melanoma specimens available from the TCGA resource¹⁴ were analyzed to identify samples with altered gene expression in comparison to reference samples using a z-score cut off of +/−2. The z-score is defined as (expression in tumor sample−mean expression in reference sample)/(standard deviation of expression in reference sample), where the reference population is either all tumors that are diploid for the gene in question, or, when available, normal adjacent tissue. Other statistical analysis details are described in FIG. 27.

Analysis of phosphorylation activity profiles. The computational methods, technical notes, and statistical tools developed to analyze the peptide phosphorylation activity profiles and kinase activity signatures are explained in the main text and supplemental information.

### REFERENCES CITED BY NUMBER IN “ADDITIONAL METHODS” SECTION AND DESCRIPTIONS OF FIGURES

1. Olow, A., et al. An Atlas of the Human Kinome Reveals the Mutational Landscape Underlying Dysregulated Phosphorylation Cascades in Cancer. Cancer research (2016).
2. Hornbeck, P. V., et al. PhosphoSitePlus, 2014: mutations, PTMs and recalibrations. Nucleic acids research 43, D512-520 (2015).
3. Prahallad, A., et al. Unresponsiveness of colon cancer to BRAF(V600E) inhibition through feedback activation of EGFR. Nature 483, 100-103 (2012).
4. Kemper, K., et al. Intra- and inter-tumor heterogeneity in a vemurafenib-resistant melanoma patient and derived xenografts. EMBO Mol Med 7, 1104-1118 (2015).
5. Kemper, K., et al. BRAF(V600E) Kinase Domain Duplication Identified in Therapy-Refractory Melanoma Patient-Derived Xenografts. Cell reports 16, 263-277 (2016).
6. Breitinger, H.-G. Drug Synergy—Mechanisms and Methods of Analysis, (InTech, 2012).
7. Chou, T. C. Drug combination studies and their synergy quantification using the Chou-Talalay method. Cancer research 70, 440-446 (2010).
8. Chou, T. C. & Talalay, P. Quantitative analysis of dose-effect relationships: the combined effects of multiple drugs or enzyme inhibitors. Adv Enzyme Regul 22, 27-55 (1984).
9. Berenbaum, M. C. Synergy, additivism and antagonism in immunosuppression. A critical review. Clin Exp Immunol 28, 1-18 (1977).
10. Foucquier, J. & Guedj, M. Analysis of drug combinations: current methodological landscape. Pharmacol Res Perspect 3, e00149 (2015).
11. Berenbaum, M. C. What is synergy? Pharmacol Rev 41, 93-141 (1989).
12. Bliss, W. L. Early Man in Western and Northwestern Canada. Science 89, 365-366 (1939).
13. Moasser, M. M., Srethapakdi, M., Sachar, K. S., Kraker, A. J. & Rosen, N. Inhibition of Src Kinases by a Selective Tyrosine Kinase Inhibitor Causes Mitotic Arrest. Cancer Res 59, 6145-6152 (1999).
14. Cancer Genome Atlas, N. Genomic Classification of Cutaneous Melanoma. Cell 161, 1681-1696 (2015).
15. Chen, Z. & Coppe, J.-P. Method and system for building and using a centralized and harmonized relational database (US2012/0296880). (United States, 2012).
16. Yang, X., et al. Widespread Expansion of Protein Interaction Capabilities by Alternative Splicing. Cell 164, 805-817 (2016).
17. Corcoran, R. B., et al. EGFR-mediated re-activation of MAPK signaling contributes to insensitivity of BRAF mutant colorectal cancers to RAF inhibition with vemurafenib. Cancer discovery 2, 227-235 (2012).
18. Brunen, D., et al. TGF-beta: an emerging player in drug resistance. Cell Cycle 12, 2960-2968 (2013).
19. Ahronian, L. G., et al. Clinical Acquired Resistance to RAF Inhibitor Combinations in BRAF-Mutant Colorectal Cancer through MAPK Pathway Alterations. Cancer discovery 5, 358-367 (2015).
20. Arao, T., et al. Small in-frame deletion in the epidermal growth factor receptor as a target for ZD6474. Cancer research 64, 9101-9104 (2004).
21. Garnett, M. J., et al. Systematic identification of genomic markers of drug sensitivity in cancer cells. Nature 483, 570-575 (2012).
22. Yang, W., et al. Genomics of Drug Sensitivity in Cancer (GDSC): a resource for therapeutic biomarker discovery in cancer cells. Nucleic acids research 41, D955-961 (2013).
23. Daemen, A., et al. Modeling precision treatment of breast cancer. Genome biology 14, R110 (2013).
24. Nazarian, R. M., Prieto, V. G., Elder, D. E. & Duncan, L. M. Melanoma biomarker expression in melanocytic tumor progression: a tissue microarray study. J Cutan Pathol 37 Suppl 1, 41-47 (2010).
25. Johannessen, C. M., et al. COT drives resistance to RAF inhibition through MAP kinase pathway reactivation. Nature 468, 968-972 (2010).
26. Girotti, M. R., et al. Paradox-breaking RAF inhibitors that also target SRC are effective in drug-resistant BRAF mutant melanoma. Cancer cell 27, 85-96 (2015).
27. Girotti, M. R., et al. Inhibiting EGF receptor or SRC family kinase signaling overcomes BRAF inhibitor resistance in melanoma. Cancer discovery 3, 158-167 (2013).
28. Sun, C., et al. Reversible and adaptive resistance to BRAF(V600E) inhibition in melanoma. Nature 508, 118-122 (2014).
29. Petricoin, E. F., Zoon, K. C., Kohn, E. C., Barrett, J. C. & Liotta, L. A. Clinical proteomics: translating benchside promise into bedside reality. Nature reviews. Drug discovery 1, 683-695 (2002).
30. Bernards, R. A missing link in genotype-directed cancer therapy. Cell 151, 465-468 (2012).

It is understood that the examples and embodiments described herein are for illustrative purposes only and that various modifications or changes in light thereof will be suggested to persons skilled in the art and are to be included within the spirit and purview of this application and scope of the appended claims. All publications, patents, accession numbers, and patent applications cited herein are hereby incorporated by reference for the purposes in the context of which they are cited.

1. A method of determining the phosphorylation activity profile of a sample comprising one or more kinases, the method comprising:
incubating the sample with a panel of sensor peptides comprising a diversity of biological sensor peptides, wherein each biological sensor peptide comprises a substrate region phosphorylated by a kinase, and the diversity of biological sensor peptides comprises biological sensor peptides for different kinases; and further, wherein members of the panel of peptides are distributed into separate reaction mixtures to assess phosphorylation activity, such that each reaction mixture represents one biological sensor peptide; and
measuring phosphorylation activity for each peptide in the separate reaction mixtures, thereby determining the phosphorylation activity profile of the sample.

2. The method of claim 1, further comprising a plurality of mutated control sensor peptides.

3. The method of claim 1, wherein the step of measuring phosphorylation activity comprises detecting ATP consumption.

4. The method of claim 1, further comprising normalizing the phosphorylation activity measured in each reaction mixture and assigning the activity to a subfamily or to a kinase based on the pattern of phosporylation activity.

5. The method of claim 1, wherein the panel comprises biological sensor peptides for kinases that are members of at least two different kinase subfamilies.

6. The method of claim 1, wherein the panel of sensor peptides comprises (a) biological sensor peptides for kinases that are members of the ABL, AKT, HER, MAPK and SFK kinase subfamilies: or (b) biological sensor peptides for kinases that are members of the ABL1, AKT1, EGFR, MAPK1/ERK2, MAPK14/p38a, HCK, and SRC subfamilies, optionally biological sensor peptides for kinases that are members of the ABL1, AKT1, EGFR, MAPK1/ERK2, MAPK14/p38a, HCK, and SRC subfamilies.

7.-8. (canceled)

9. The method of claim 1, wherein the panel comprises at least three biological sensor peptides for each kinase; or biological sensor peptides for kinases that are members of the ABL1, AKT1, EGFR, MAPK1/ERK2, MAPK14/p38a, HCK, and SRC subfamilies.

10. (canceled)

11. The method of claim 1, wherein the individual sensor peptides are 9 to 21 amino acids in length.

12.-13. (canceled)

14. The method of claim 1, wherein the panel comprises at least 10, at least 20, at least 30, at least 40, at least 50, at least 60, at least 70, at least 80, or at least 90 biological sensor peptides having a sequence as shown in Table 1 for the peptide id of a biological sensor peptide shown in FIG. 4: or the panel comprises at least 100 sensor peptides, or at least 110, at least 120, at least 130, at least 140, or 151 biological sensor peptides having a sequence as shown in Table 1 for the peptide id of a biological sensor peptide shown in FIG. 4.

15. (canceled)

16. The method of claim 15, wherein the panel comprises at least 10, at least 20, at least 30, at least 40, at least 50, at least 60, or 63 reference peptides as identified in FIG. 4, optionally wherein the panel comprises the 228 peptides as identified in FIG. 4.

17. (canceled)

18. The method of claim 1, wherein the sample comprises cancer cells.

19. The method of claim 18, wherein the cancer cells are from a solid tumor; optionally wherein the solid tumor is a breast tumor, a colorectal tumor, a melanoma, or a lung tumor.

20. (canceled)

21. The method of claim 19, wherein the sample comprises metastatic cancer cells.

22. The method of claim 18, wherein the cancer cells are from a hematological cancer.

23. The method of claim 18, wherein the cancer cells are from a patient that has been treated with a cancer therapeutic agent.

24. The method of claim 23, wherein the therapeutic agent is a kinase inhibitor.

25. (canceled)

26. A panel comprising a plurality of biological sensor peptides to evaluate phosphorylation activity of two or more different kinases.

27. The panel of claim 26, further comprising a plurality of control mutated sensor peptides.

28. The panel of claim 26, wherein the panel comprises 4, 5, 6, 7, 8, 9, or 10 biological sensor peptide for each kinase.

29. (canceled)

30. A panel of biological sensor peptides to evaluate phosphorylation activity, wherein the panel comprises at least four biological sensor peptides for each of kinase families ABL, AKT, HER, MAPK and SFK, optionally, wherein the panel of sensor peptides comprises at least four biological sensor peptides for each of kinase subfamilies ABL1, AKT1, EGFR, MAPK1/ERK2, MAPK14/p38a, HCK, and SRC.

31. (canceled)

32. The panel of claim 30, wherein the panel of sensor peptides comprises at least three biological sensor peptides for each kinase BLK, BRK, FGR, FRK, HCK, LCK, LYN A, SRMS, YES1, ABL1T315I, EGFR, JAK2, CSK, AKT1, AKT2, AKT3, MAPK1/ERK2, and MAPK14/p38a.

33. The panel of claim 32, wherein the panel comprises 4, 5, 6, 7, 8, 9, or 10 biological sensor peptide for each kinase, optionally wherein the individual sensor peptides are 9 to 21 amino acids in length.

34.-36. (canceled)

37. The panel of claim 30, wherein the panel comprises at least 10, at least 20, at least 30, at least 40, at least 50, at least 60, at least 70, at least 80, or at least 90 biological sensor peptides having a sequence as shown in Table 1 for the peptide id of a biological sensor peptide shown in FIG. 4, optionally wherein the panel comprises at least 100 sensor peptides, or at least 110, at least 120, at least 130, at least 140, or 151 biological sensor peptides having a sequence as shown in Table 1 for the peptide id of a biological sensor peptide shown in FIG. 4.

38. The panel of claim 37, wherein the panel comprises at least 100 sensor peptides, or at least 110, at least 120, at least 130, at least 140, or 151 biological sensor peptides having a sequence as shown in Table 1 for the peptide id of a biological sensor peptide shown in FIG. 4; optionally wherein the panel comprises at least 10, at least 20, at least 30, at least 40, at least 50, at least 60, or 63 reference peptides as identified in FIG. 4.

39. (canceled)

40. The panel of claim 38, wherein the panel comprises the 228 peptides shown in FIG. 4.
